(19) United States
(12) Patent Application Publication
(10) Pub. No.: US 2021/0253582 A1
(43) Pub. Date: Aug. 19, 2021

(54) SALTS AND SOLID FORMS AND PROCESSES OF PREPARING A PI3K INHIBITOR

(71) Applicant: Incyte Corporation, Wilmington, DE (US)

(72) Inventors: Shili Chen, Newark, DE (US); Zhongjiang Jia, Kennett Square, PA (US); Yi Li, Newark, DE (US); Yongchun Pan, Wilmington, DE (US); Jiacheng Zhou, Newark, DE (US)

(21) Appl. No.: 17/169,008

(22) Filed: Feb. 5, 2021

Related U.S. Application Data
(60) Provisional application No. 62/970,985, filed on Feb. 6, 2020.

(51) Int. Cl. C07D 487/04

(52) U.S. Cl. CPC C07D 487/04; C07B 2200/13

(57) ABSTRACT

The present disclosure provides processes for preparing (R)-4-(3-((S)-1-(4-amino-3-methyl-1H-pyrazolo[3,4-d]pyrimidin-1-yl)ethyl)-5-chloro-2-ethoxy-6-fluorophenyl)pyrrolidin-2-one, which is useful as an inhibitor phosphoinositide 3-kinase-delta (PI3Kδ), as well as a salt form and intermediates related thereto.

### TECHNICAL FIELD

The present disclosure provides processes for preparing (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one, which is useful as an inhibitor of phosphoinositide 3-kinase-delta (PI3Kδ), as well as salt forms and intermediates related thereto.

### BACKGROUND

U.S. Pat. No. 10,092,570 and WO2013/0333569 describe heterocyclylamine derivatives that are useful as inhibitors of phosphoinositide 3-kinase-delta (PI3Kδ). One such inhibitor is (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one. The two above mentioned patent documents provide select methods of preparing the PI3Kδ inhibitors.

There is a need to develop new processes for preparing PI3Kδ inhibitors, and related intermediates and salt forms. This invention is directed to this need and others.

### SUMMARY

The present disclosure provides processes of preparing a compound of Formula I:

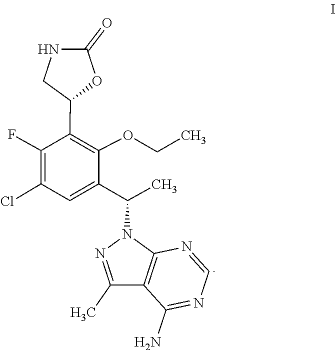 [SMILES: CCOc1c([C@H](C)n2nc(C)c3c(N)ncnc32)cc(Cl)c(F)c1[C@@H]1CNC(=O)O1]

which is useful as an inhibitor of PI3Kδ.

The present disclosure further provides crystalline salts and free base solid forms of the compound of Formula I.

The present disclosure also provides pharmaceutical compositions comprising a salt or free base solid form described herein and a pharmaceutically acceptable carrier.

The present disclosure further provides methods of inhibiting an activity of a PI3K kinase, comprising contacting the kinase with a salt or free base solid form of the compound of Formula I.

The present disclosure also provides methods of treating a disease in a patient, wherein said disease is associated with abnormal expression or activity of a PI3K kinase, comprising administering to said patient a therapeutically effective amount of a salt or free base solid form of the compound of Formula I.

The present disclosure additionally provides the salts and free base solid form of the compound of Formula I for use in any of the methods described herein.

The present disclosure further provides use of the salts and free base solid form of the compound of Formula I for the manufacture of a medicament for use in any of the methods described herein.

The present disclosure also provides a process of preparing the hydrochloric acid salt of the compound of Formula I, comprising reacting a compound of Formula I:

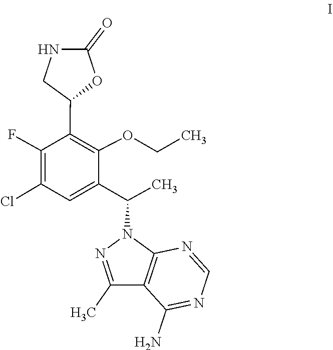 [SMILES: CCOc1c([C@H](C)n2nc(C)c3c(N)ncnc32)cc(Cl)c(F)c1[C@@H]1CNC(=O)O1]

with hydrochloric acid to form said salt.

The present disclosure also provides a process of preparing the phosphoric acid salt of the compound of Formula I, comprising reacting a compound of Formula I with phosphoric acid to form said salt.

The present disclosure also provides a process of preparing the maleic acid salt of the compound of Formula I, comprising reacting a compound of Formula I with maleic acid to form said salt.

The present disclosure also provides a process of preparing the p-toluenesulfonic acid salt of the compound of Formula I, comprising reacting a compound of Formula I with p-toluenesulfonic acid to form said salt.

The present disclosure additionally provides a process of preparing a compound of Formula IA

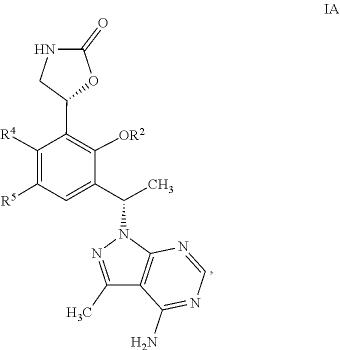

comprising reacting a compound of Formula XIV:

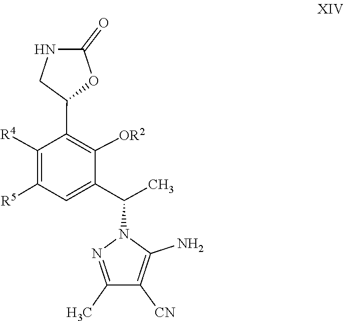

with formamidine acetate to form said compound of Formula IA; wherein:

R² is C₁₋₆ alkyl;

R⁴ is halo, CN, or C₁₋₃ alkyl; and

R⁵ is halo, CN, or C₁₋₃ alkyl.

In some embodiments, R² is methyl or ethyl.

In some embodiments, R² is ethyl.

In some embodiments, R⁴ is F, Cl, CN, or methyl.

In some embodiments, R⁴ is F.

In some embodiments, R⁵ is Cl, CN, or methyl.

In some embodiments, R⁵ is Cl.

In some embodiments, R² is ethyl, R⁴ is F, and R⁵ is Cl.

### DESCRIPTION OF DRAWINGS

FIG. 1 shows an XRPD pattern representative of a hydrochloric acid salt of the compound of Formula I.

FIG. 2 shows a DSC thermogram representative of a hydrochloric acid salt of the compound of Formula I.

FIG. 3 shows TGA data representative of a hydrochloric acid salt of the compound of Formula I.

FIG. 4 shows an XRPD pattern representative of a phosphoric acid salt of the compound of Formula I.

FIG. 5 shows a DSC thermogram representative of a phosphoric acid salt of the compound of Formula I.

FIG. 6 shows TGA data representative of a phosphoric acid salt of the compound of Formula I.

FIG. 7 shows an XRPD pattern representative of a maleic acid salt of the compound of Formula I.

FIG. 8 shows a DSC thermogram representative of a maleic acid salt of the compound of Formula I.

FIG. 9 shows TGA data representative of a maleic acid salt of the compound of Formula I.

FIG. 10 shows an XRPD pattern representative of a p-toluenesulfonic acid salt of the compound of Formula I.

FIG. 11 shows a DSC thermogram representative of a p-toluenesulfonic acid salt of the compound of Formula I.

FIG. 12 shows TGA data representative of a p-toluenesulfonic acid salt of the compound of Formula I.

FIG. 13 shows an XRPD pattern representative of the crystalline free base of the compound of Formula I.

FIG. 14 shows a DSC thermogram representative of the crystalline free base of the compound of Formula I.

FIG. 15 shows TGA data representative of the crystalline free base of the compound of Formula I.

FIG. 16 shows the asymmetric crystalline unit of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one hydrochloric acid salt, acetone solvate form, with thermal ellipsoids drawn to the 30% probability level.

FIGS. 17 and 18 each show one molecule of the (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one hydrochloric acid salt, acetone solvate form that makes up the bimolecular asymmetric crystalline unit shown in FIG. 16. Thermal ellipsoids are drawn to the 30% probability level.

### DETAILED DESCRIPTION

### Compounds and Salts

The present disclosure relates to a compound of Formula I.

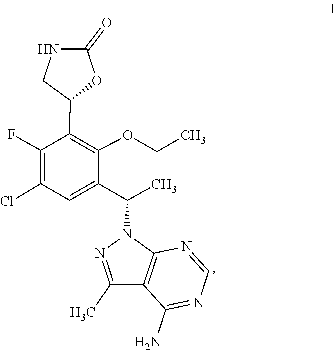 [SMILES: CCOc1c([C@H](C)n2nc(C)c3c(N)ncnc32)cc(Cl)c(F)c1[C@@H]1CNC(=O)O1]

which is useful as an inhibitor of PI3Kδ.

The present disclosure provides salts of the compound of Formula I.

Accordingly, in some embodiments, the present disclosure provides 5-(3-(1-(4-amino-3-methyl-1H-pyrazolo[3,4-d]pyrimidin-1-yl)ethyl)-5-chloro-2-ethoxy-6-fluorophenyl)oxazolidin-2-one hydrochloric acid salt, phosphoric acid salt, maleic acid salt, and p-toluenesulfonic acid salt. In some embodiments, the present disclosure provides (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one hydrochloric acid salt, phosphoric acid salt, maleic acid salt, and p-toluenesulfonic acid salt.

In some embodiments, the hydrochloric acid salt is a 1:1 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to hydrochloric acid.

In some embodiments, the phosphoric acid salt is a 5:4 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to phosphoric acid.

In some embodiments, the maleic acid salt is a 1:1 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to maleic acid.

In some embodiments, the p-toluenesulfonic acid salt is a 1:1 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to p-toluenesulfonic acid.

In some embodiments, the salts of the compound of Formula I provided herein is crystalline. As used herein, “crystalline” or “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content.

The present disclosure further provides crystalline forms of the free base of the compound of Formula I. In some embodiments, the present disclosure provides crystalline 5-(3-(1-(4-amino-3-methyl-1H-pyrazolo[3,4-d]pyrimidin-1-yl)ethyl)-5-chloro-2-ethoxy-6-fluorophenyl)oxazolidin-2-one free base. In some embodiments, the present disclosure provides crystalline (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one free base.

Different forms of the same substance have different bulk properties relating to, for example, hygroscopicity, solubility, stability, and the like. Forms with high melting points often have good thermodynamic stability which is advantageous in prolonging shelf-life drug formulations containing the solid form. Forms with lower melting points often are less thermodynamically stable, but are advantageous in that they have increased water solubility, translating to increased drug bioavailability. Forms that are weakly hygroscopic are desirable for their stability to heat and humidity and are resistant to degradation during long storage.

The different free base solid forms and salt solid forms can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

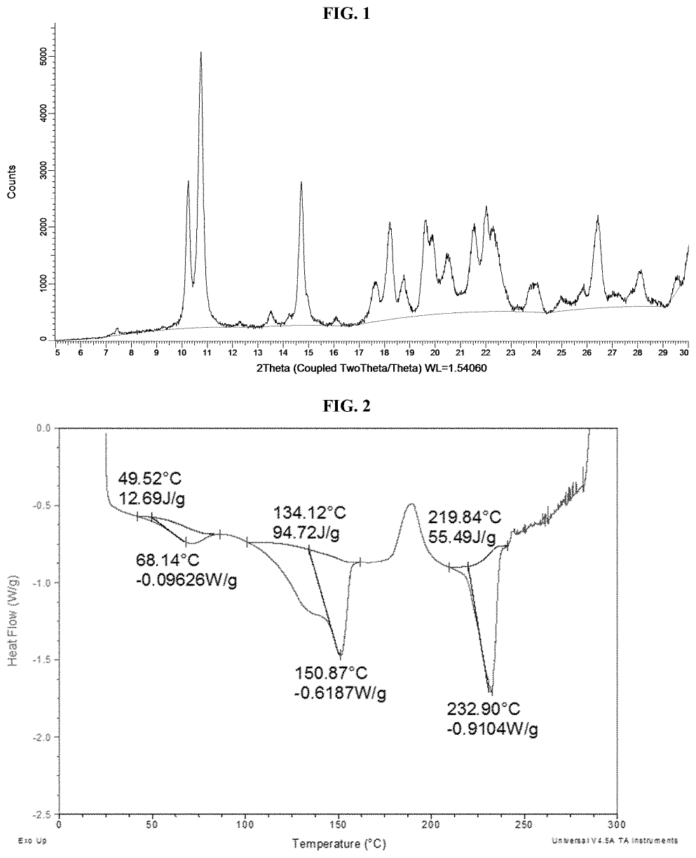

In some embodiments, the hydrochloric acid salt of the compound of Formula I has at least one XRPD peak, in terms of 2-theta, selected from about 10.2°, about 10.7°, about 14.7°, about 18.2°, about 19.6°, about 19.9°, about 20.5°, about 21.5°, about 22.0°, about 22.3°, and about 26.4°. In some embodiments, the hydrochloric acid salt of the compound of Formula I has at least two of said XRPD peaks, at least three of said XRPD peaks, at least four of said XRPD peaks, or at least five of said XRPD peaks. In some embodiments, the hydrochloric acid salt of the compound of Formula I has XRPD peaks, in terms of 2-theta, at each of about 10.2°, about 10.7°, about 14.7°, about 18.2°, about 19.6°, about 19.9°, about 20.5°, about 21.5°, about 22.0°, about 22.3°, and about 26.4°. In some embodiments, the hydrochloric acid salt of the compound of Formula I has an XRPD profile substantially as shown in FIG. 1.

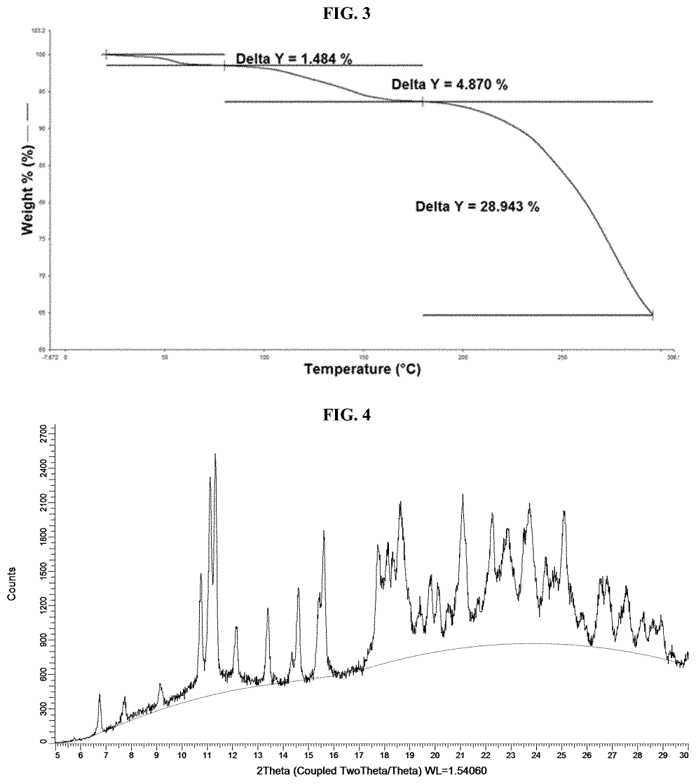

In some embodiments, the hydrochloric acid salt of the compound of Formula I has a DSC thermogram having at least one endothermic peak at a temperature selected from about 68.1° C., about 150.9° C., and about 232.9° C. In some embodiments, the hydrochloric acid salt of the compound of Formula I has a DSC thermogram having endothermic peaks at about 68.1° C., about 150.9° C., and about 232.9° C. In some embodiments, the hydrochloric acid salt of the compound of Formula I has a DSC thermogram having an exothermic peak at about 175° C. to about 225° C. In some embodiments, the hydrochloric acid salt of the compound of Formula I has a DSC thermogram substantially as shown in FIG. 2. In some embodiments, the hydrochloric acid salt of the compound of Formula I has a TGA thermogram substantially as shown in FIG. 3. In some embodiments, the hydrochloric acid salt of the compound of Formula I has a TGA thermogram showing weight loss greater than 20% above temperatures of about 180° C.

In some embodiments, the phosphoric acid salt of the compound of Formula I has at least one XRPD peak, in terms of 2-theta, selected from about 11.10, about 11.3°, about 15.6°, about 17.7°, about 18.1°, about 18.3°, about 18.6°, about 21.1°, about 22.3°, about 22.9°, about 23.5°, about 23.7°, and about 25.1°. In some embodiments, the phosphoric acid salt of the compound of Formula I has at least two of said XRPD peaks, at least three of said XRPD peaks, at least four of said XRPD peaks, or at least five XRPD peaks. In some embodiments, the phosphoric acid salt of the compound of Formula I has XRPD peaks, in terms of 2-theta, at each of about 11.10, about 11.3°, about 15.6°, about 17.7°, about 18.1°, about 18.3°, about 18.6°, about 21.1°, about 22.3°, about 22.9°, about 23.5°, about 23.7°, and about 25.1°. In some embodiments, the phosphoric acid salt of the compound of Formula I has an XRPD profile substantially as shown in FIG. 4.

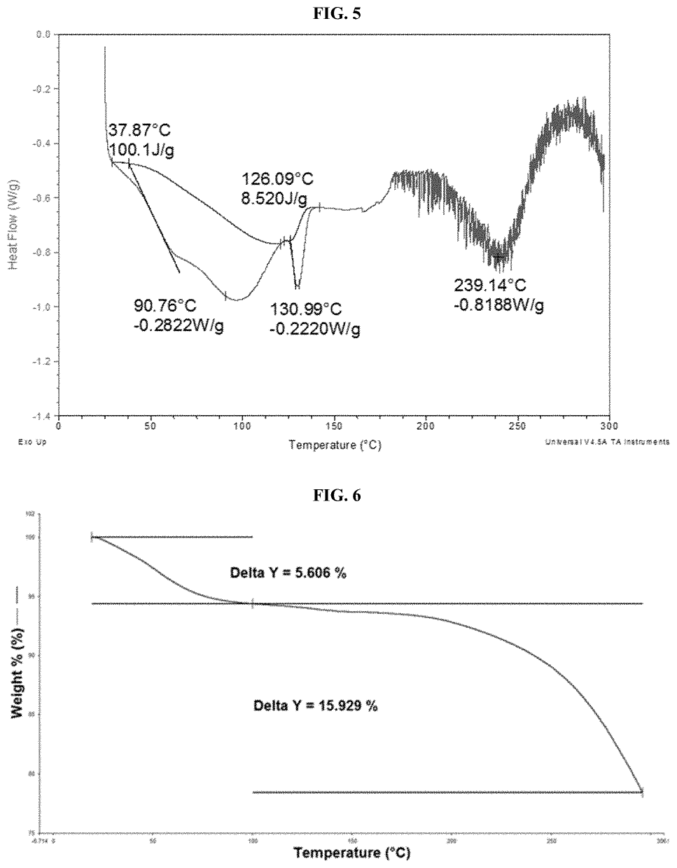

In some embodiments, the phosphoric acid salt of the compound of Formula I has a DSC thermogram having at least one endothermic peak at a temperature selected from about 90.8° C., about 131.0° C., and about 239.1° C. In some embodiments, the phosphoric acid salt of the compound of Formula I has a DSC thermogram having endothermic peaks at about 90.8° C., about 131.0° C., and about 239.1° C. In some embodiments, the phosphoric acid salt of the compound of Formula I has a DSC thermogram substantially as shown in FIG. 5. In some embodiments, the phosphoric acid salt of the compound of Formula I has a TGA thermogram substantially as shown in FIG. 6. In some embodiments, the phosphoric acid salt of the compound of Formula I has a TGA thermogram showing weight loss greater than 20% above temperatures of about 100° C.

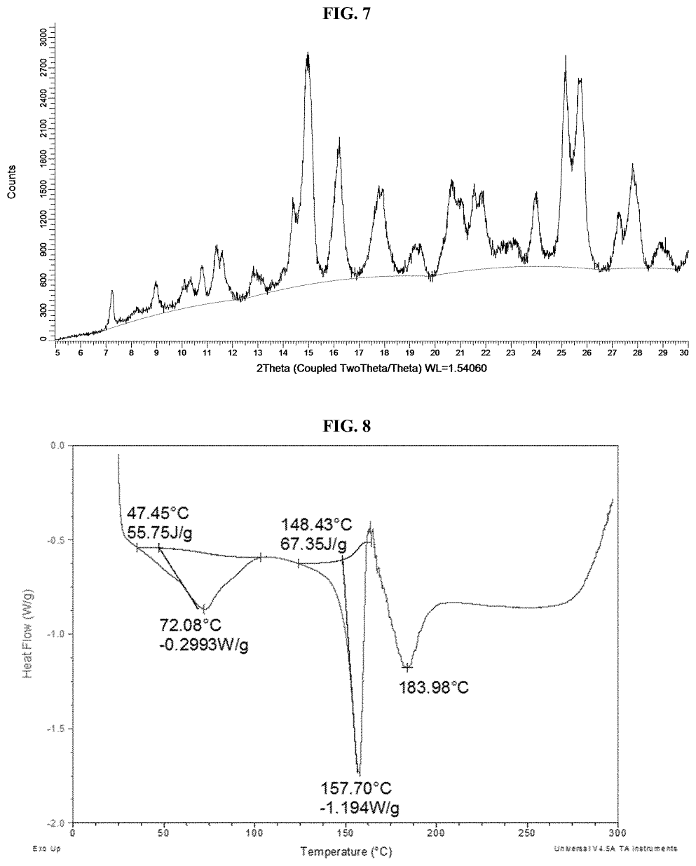

In some embodiments, the maleic acid salt of the compound of Formula I has at least one XRPD peak, in terms of 2-theta, selected from about 11.10, about 11.3°, about 15.6°, about 17.7°, about 18.1°, about 18.3°, about 18.6°, about 21.1°, about 22.3°, about 22.9°, about 23.5°, about 23.7°, and about 25.1°. In some embodiments, the maleic acid salt of the compound of Formula I has at least two of said XRPD peaks, at least three of said XRPD peaks, at least four of said XRPD peaks, or at least five of said XRPD peaks. In some embodiments, the maleic acid salt of the compound of Formula I has XRPD peaks, in terms of 2-theta, at each of about 11.10, about 11.3°, about 15.6°, about 17.7°, about 18.1°, about 18.3°, about 18.6°, about 21.1°, about 22.3°, about 22.9°, about 23.5°, about 23.7°, and about 25.1°. In some embodiments, the maleic acid salt of the compound of Formula I has an XRPD profile substantially as shown in FIG. 7.

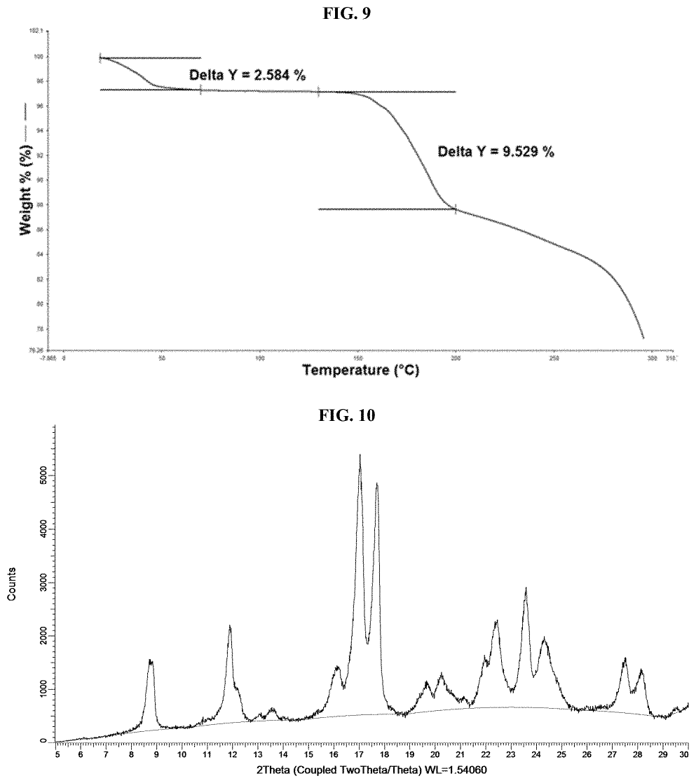

In some embodiments, the maleic acid salt of the compound of Formula I has a DSC thermogram having at least one endothermic peak at a temperature selected from about 72.1° C., about 157.7° C., and about 184.0° C. In some embodiments, the maleic acid salt of the compound of Formula I has a DSC thermogram having endothermic peaks at about 72.1° C., about 157.7° C., and about 184.0° C. In some embodiments, the maleic acid salt of the compound of Formula I has a DSC thermogram substantially as shown in FIG. 8. In some embodiments, the maleic acid salt of the compound of Formula I has a TGA thermogram substantially as shown in FIG. 9. In some embodiments, the maleic acid salt of the compound of Formula I has a TGA thermogram showing weight loss greater than 20% above temperatures of about 200° C.

In some embodiments, the p-toluenesulfonic acid salt of the compound of Formula I has at least one XRPD peak, in terms of 2-theta, selected from about 8.8°, about 11.9°, about 17.0°, about 17.7°, about 22.4°, about 23.6°, and about 24.3°. In some embodiments, the p-toluenesulfonic acid salt of the compound of Formula I has at least two of said XRPD peaks, at least three of said XRPD peaks, at least four of said XRPD peaks, or at least five of said XRPD peaks. In some embodiments, the p-toluenesulfonic acid salt of the compound of Formula I has XRPD peaks, in terms of 2-theta, at each of about 8.8°, about 11.9°, about 17.0°, about 17.7°, about 22.4°, about 23.6°, and about 24.3°. In some embodiments, the p-toluenesulfonic acid salt of the compound of Formula I has an XRPD profile substantially as shown in FIG. 10.

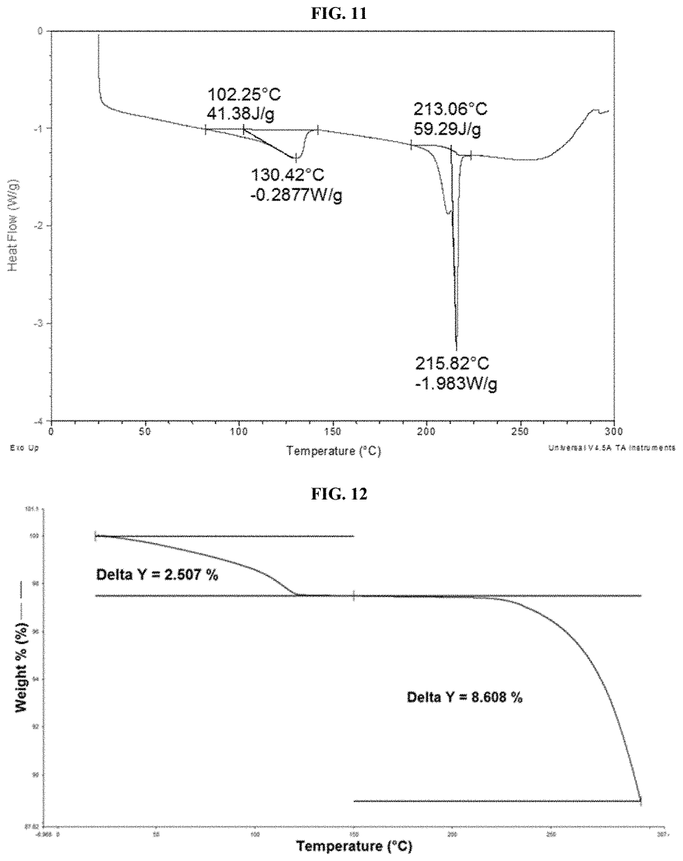

In some embodiments, the p-toluenesulfonic acid salt of the compound of Formula I has a DSC thermogram having at least one endothermic peak at a temperature selected from about 72.1° C., about 157.7° C., and about 184.0° C. In some embodiments, the p-toluenesulfonic acid salt of the compound of Formula I has a DSC thermogram having endothermic peaks at about 72.1° C., about 157.7° C., and about 184.0° C. In some embodiments, the p-toluenesulfonic acid salt of the compound of Formula I has a DSC thermogram substantially as shown in FIG. 11. In some embodiments, the p-toluenesulfonic acid salt of the compound of Formula I has a TGA thermogram substantially as shown in FIG. 12. In some embodiments, the p-toluenesulfonic acid salt of the compound of Formula I has a TGA thermogram showing weight loss greater than 20% above temperatures of about 200° C.

In some embodiments, the crystalline free base of the compound of Formula I has at least one XRPD peak, in terms of 2-theta, selected from about 9.2°, about 11.5°, about 14.2°, about 15.10, about 20.3°, about 20.7°, about 21.4°, about 23.0°, and about 27.6°.

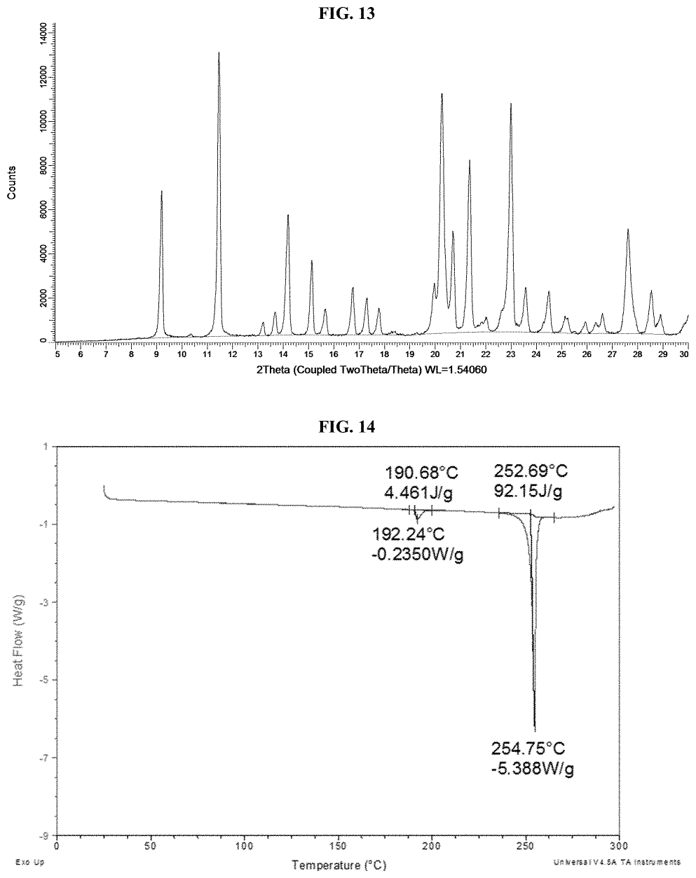

In some embodiments, the crystalline free base of the compound of Formula I has at least two of said XRPD peaks, at least three of said XRPD peaks, at least four of said XRPD peaks, or at least five of said XRPD peaks. In some embodiments, the crystalline free base of the compound of Formula I has XRPD peaks, in terms of 2-theta, at each of about 9.2°, about 11.5°, about 14.2°, about 15.10, about 20.3°, about 20.7°, about 21.4°, about 23.0°, and about 27.6°. In some embodiments, the crystalline free base of the compound of Formula I has an XRPD profile substantially as shown in FIG. 13.

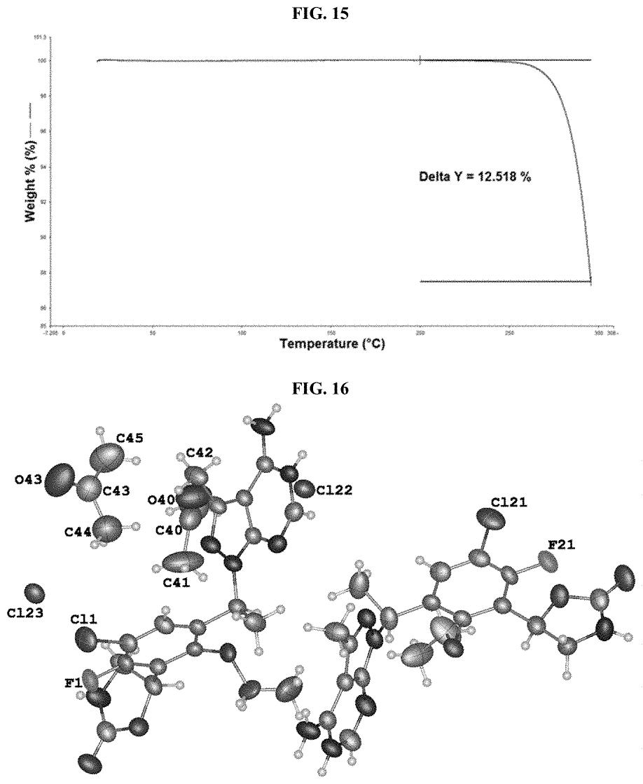

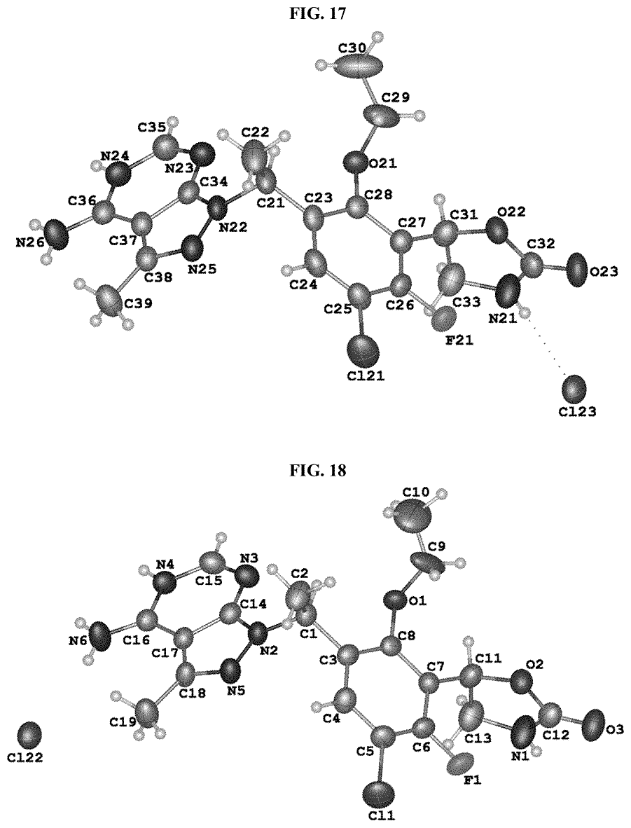

In some embodiments, the crystalline free base of the compound of Formula I has a DSC thermogram having at least one endothermic peak at a temperature selected from about 192.2° C., and about 254.8° C. In some embodiments, the crystalline free base of the compound of Formula I has a DSC thermogram having endothermic peaks at about 192.2° C., and about 254.8° C. In some embodiments, the crystalline free base of the compound of Formula I has a DSC thermogram substantially as shown in FIG. 14. In some embodiments, the crystalline free base of the compound of Formula I has a TGA thermogram substantially as shown in FIG. 15. In some embodiments, the crystalline free base of the compound of Formula I has a TGA thermogram showing weight loss greater than 20% above temperatures of about 200° C.

In some embodiments, the salts and free base compounds described herein (e.g., the free base compound of Formula I or the hydrochloric acid salt, phosphoric acid salt, maleic acid salt or p-toluenesulfonic acid salt of the compound of Formula I) are substantially isolated. By “substantially isolated” is meant that the salt or compound is at least partially or substantially separated from the environment in which it was formed or detected. Partial separation can include, for example, a composition enriched in the compounds and salts described herein. Substantial separation can include compositions containing at least about 50%, at least about 60%, at least about 70%, at least about 80%, at least about 90%, at least about 95%, at least about 97%, or at least about 99% by weight of the compounds and salts described herein.

### Intermediates

The present disclosure further provides intermediates that are useful in the preparation of the compound of Formula I and more generally compounds of Formula Ia.

Accordingly, in some embodiments, the present disclosure provides a compound

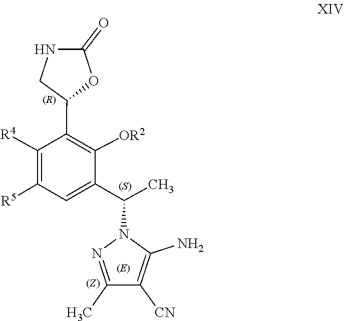

or a pharmaceutically acceptable salt thereof.

The present disclosure further provides a compound of Formula XIII:

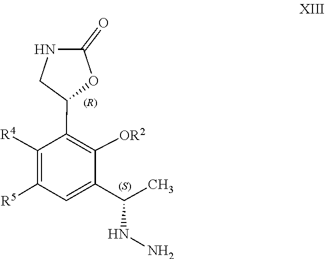

or a pharmaceutically acceptable salt thereof.

The present disclosure further provides a compound of Formula XII:

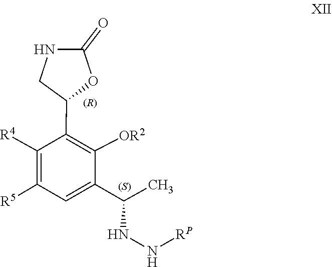

or a pharmaceutically acceptable salt thereof, wherein R^{p} is an amine protecting group.

The present disclosure further provides a compound of Formula XI:

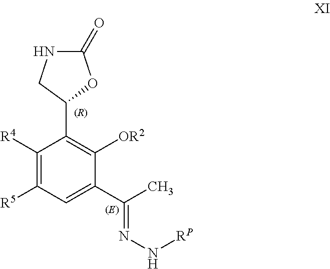

or a pharmaceutically acceptable salt thereof, wherein R^{p} is an amine protecting group.

The present disclosure further provides a compound of Formula X:

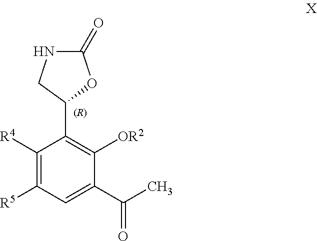

or a pharmaceutically acceptable salt thereof.

The present disclosure further provides a compound of Formula IX:

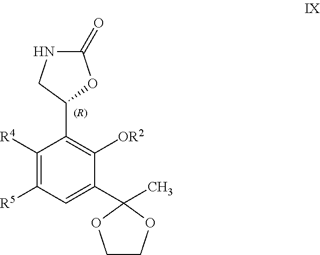

or a pharmaceutically acceptable salt thereof.

The present disclosure further provides a compound of Formula VIII:

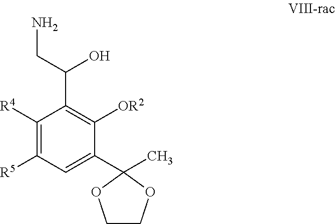

or a pharmaceutically acceptable salt thereof.

The present disclosure further provides a compound of Formula VIII:

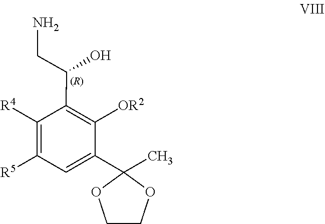

or a pharmaceutically acceptable salt thereof.

The present disclosure further provides a compound of Formula VII:

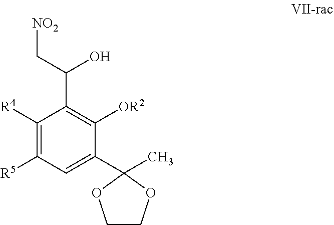

or a pharmaceutically acceptable salt thereof.

The present disclosure further provides a compound of Formula VII:

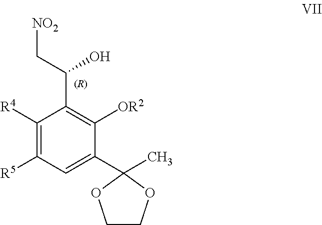

or a pharmaceutically acceptable salt thereof.

Individually, in each of the compounds of Formulas VII-XIV, VII-rac and VIII-rac:

R² is C₁₋₆ alkyl;

R⁴ is halo, CN, or C₁₋₃ alkyl; and

R⁵ is halo, CN, or C₁₋₃ alkyl.

In some embodiments, R² is methyl or ethyl.

In some embodiments, R² is ethyl.

In some embodiments, R⁴ is F, Cl, CN, or methyl.

In some embodiments, R⁴ is F.

In some embodiments, R⁵ is Cl, CN, or methyl.

In some embodiments, R⁵ is Cl.

In some embodiments, R² is ethyl, R⁴ is F, and R⁵ is Cl.

### Processes

The present disclosure further provides a process of preparing a salt of the compound of Formula I.

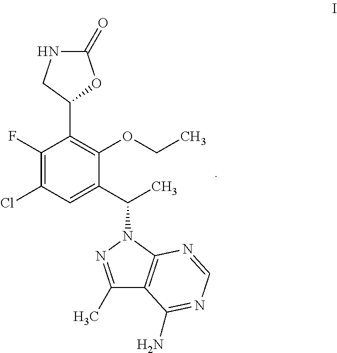 [SMILES: CCOc1c([C@H](C)n2nc(C)c3c(N)ncnc32)cc(Cl)c(F)c1[C@@H]1CNC(=O)O1]

In some embodiments, the process of preparing the salt of the compound of Formula I comprises reacting a compound of Formula I with an acid to form said salt.

In some embodiments, the acid is selected from hydrochloric acid, phosphoric acid, maleic acid, and p-toluenesulfonic acid.

In some embodiments, the acid is hydrochloric acid.

In some embodiments, said hydrochloric acid is 1 M aqueous hydrochloric acid.

In some embodiments, about 1.0 to about 1.5 equivalents of hydrochloric acid is used based on 1 equivalent of the compound of Formula I.

In some embodiments, said reacting with the hydrochloric acid is performed at about room temperature.

In some embodiments, the acid is phosphoric acid.

In some embodiments, said phosphoric acid is 1 M aqueous phosphoric acid.

In some embodiments, about 1.0 to about 1.5 equivalents of phosphoric acid is used based on 1 equivalent of the compound of Formula I.

In some embodiments, said reacting with the phosphoric acid is performed at about room temperature.

In some embodiments, the acid is maleic acid.

In some embodiments, about 1.0 to about 1.5 equivalents of maleic acid is used based on 1 equivalent of the compound of Formula I.

In some embodiments, said reacting with the maleic acid is performed at about room temperature.

In some embodiments, the acid is p-toluenesulfonic acid.

In some embodiments, about 1.0 to about 1.5 equivalents of p-toluenesulfonic acid is used based on 1 equivalent of the compound of Formula I.

In some embodiments, said reacting with the p-toluenesulfonic acid is performed at about room temperature.

The present disclosure further provides a process of preparing a compound of Formula IA:

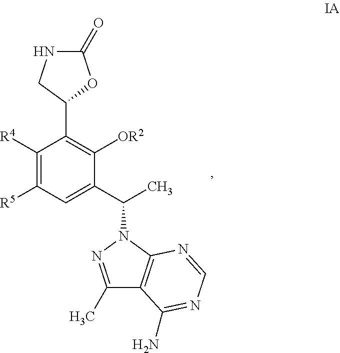

wherein:

R² is C₁₋₆ alkyl;

R⁴ is halo, CN, or C₁₋₃ alkyl; and

R⁵ is halo, CN, or C₁₋₃ alkyl.

In some embodiments, R² is methyl or ethyl.

In some embodiments, R² is ethyl.

In some embodiments, R⁴ is F, Cl, CN, or methyl.

In some embodiments, R⁴ is F.

In some embodiments, R⁵ is Cl, CN, or methyl.

In some embodiments, R⁵ is Cl.

In some embodiments, R² is ethyl, R⁴ is F, and R⁵ is Cl.

In some embodiments, the compound of Formula IA is the compound of Formula I.

In some embodiments, the process of preparing a compound of Formula IA comprises reacting a compound of Formula XIV:

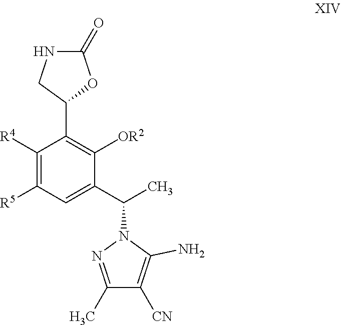

with formamidine acetate to form said compound of Formula IA; wherein R², R⁴, and R⁵ are as described above.

In some embodiments, said reacting of the compound of Formula XIV with formamidine acetate is performed in a solvent component comprising isopropyl acetate, n-propyl acetate, and bis(2-methoxyethyl) ether (also known as diglyme).

In some embodiments, said reacting of the compound of Formula XIV with formamidine acetate is performed at a temperature of from about 110° C. to about 120° C.

In some embodiments, about 5 to about 10 equivalents of formamidine acetate is used based on 1 equivalent of the compound of Formula XIV.

In some embodiments, said compound of Formula XIV is prepared by a process comprising reacting a compound of Formula XIII:

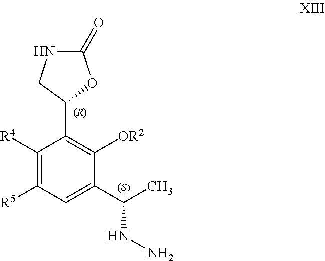

or a pharmaceutically acceptable salt thereof, with (1-ethoxyethylidene)malononitrile; wherein R², R⁴, and R⁵ are as described above.

In some embodiments, said reacting takes place in the presence of a base.

In some embodiments, said base is potassium carbonate.

In some embodiments, said reacting of the compound of Formula XIII with (1-ethoxyethylidene)malononitrile is performed at about room temperature.

In some embodiments, said compound of Formula XIII is prepared by a process comprising deprotecting a compound of Formula XII:

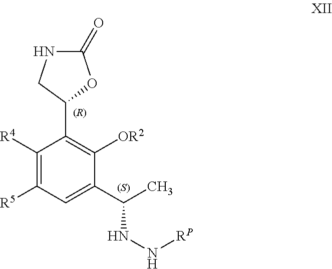

or a pharmaceutically acceptable salt thereof, with wherein:

R^{p} is an amine protecting group; and

R², R⁴, and R⁵ are as described above.

Amino protecting groups R^{p} may be used to prevent unwanted reactions of an amino group while performing a desired transformation. Amino protecting groups allow easy covalent attachment to a nitrogen atom as well as selective cleavage from the nitrogen atom. Suitable “amino protecting groups”, such as alkoxycarbonyl (such as ethoxycarbonyl, tert-butoxycarbonyl (Boc), benzyloxycarbonyl (Cbz), 9-fluorenylmethyloxycarbonyl (Fmoc), and the like), acyl (such as acetyl (Ac), benzoyl (Bz), and the like), sulfonyl (such as methanesulfonyl, trifluoromethanesulfonyl, and the like), arylalkyl (such as benzyl, 4-methoxybenzyl, diphenylmethyl, triphenylmethyl (trityl), and the like), alkenylalkyl (such as allyl, prenyl, and the like), diarylmethyleneyl (such as (C₆H₅)₂C═N, and the like), and silyl (such as tert-butyldimethylsilyl, triisopropylsilyl, and the like), are known to one skilled in the art. The chemistry of amino protecting groups can be found in Wuts and Greene, Greene's Protective Groups in Organic Synthesis, 4^{th} Ed., pp 696-926, John Wiley & Sons: New York, 2006, which is incorporated herein by reference in its entirety.

In some embodiments, amino deprotecting agents useful for deprotecting a compound of Formula XII are known to those skilled in the art, such as those in Wuts and Greene (supra). In particular, the amino protecting groups described above can be conveniently removed using many available amino deprotecting agents that are specific to the various groups mentioned above without affecting other desired portions of the compound. The tert-butoxycarbonyl group can be removed (e.g., hydrolyzed) from the nitrogen atom, for example, by treatment with an acid (such as hydrochloric acid, trifluoroacetic acid, toluenesulfonic acid, and the like); a combination of reagents (e.g., mixture of acetyl chloride and methanol) known to generate an acid; or a Lewis acid (e.g., BF₃ Et₂O). The benzyloxycarbonyl group can be removed (e.g., hydrogenolyzed) from the nitrogen atom, for example, by treatment with hydrogen and a catalyst (such as palladium on carbon).

In some embodiments, R^{p} is tert-butoxycarbonyl (Boc).

In some embodiments, the deprotecting comprises reacting the compound of Formula XII with an acid.

In some embodiments, the deprotecting comprises reacting the compound of Formula XII with hydrochloric acid.

In some embodiments, the hydrochloric acid is 1N aqueous hydrochloric acid.

In some embodiments, the deprotecting takes place in the presence of a solvent component.

In some embodiments, the solvent component comprises trifluoroethanol and isopropanol.

In some embodiments, the deprotecting is performed at a temperature of from about 45° C. to about 55° C.

In some embodiments, said compound of Formula XII is prepared by a process comprising reacting a compound of Formula XI:

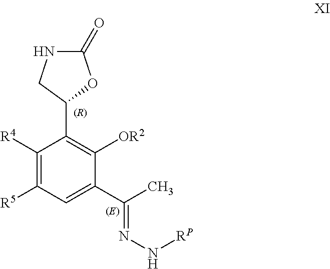

with hydrogen gas in the presence of one or more independently selected hydrogenation catalysts, wherein R, R², R⁴, and R⁵ are as described above.

In some embodiments, said hydrogenation catalyst is a rhodium catalyst.

In some embodiments, said hydrogenation catalyst is one or more of bis(norbornadiene)rhodium(I) tetrafluoroborate and bis(1,5-cyclooctadiene)rhodium(I) tetrafluoroborate.

In some embodiments, said hydrogenation catalyst further comprises a supporting ligand.

In some embodiments, said supporting ligand is one or more of (R)-(−)-1-{(S)-2-[bis(4-trifluoromethylphenyl)phosphine]ferrocenyl}ethyl-di-t-butylphosphine and 2,3-bis((S)-tert-butyl(methyl)phosphanyl)quinoxaline.

In some embodiments, about 0.08 to about 0.12 equivalents of hydrogenation catalyst is used based on 1 equivalents of the compound of Formula XI.

In some embodiments, about 0.08 to about 0.12 equivalents of hydrogenation catalyst and supporting ligand is used based on 1 equivalents of the compound of Formula XI.

In some embodiments, said reacting of the compound of Formula XI with hydrogen gas is performed at about room temperature.

In some embodiments, said reacting of the compound of Formula XI with hydrogen gas is performed at a temperature of from about 65° C. to about 75° C.

In some embodiments, said reacting of the compound of Formula XI with hydrogen gas is performed at a hydrogen pressure of about 55-60 psi of hydrogen atmosphere.

In some embodiments, said reacting of the compound of Formula XI with hydrogen gas is performed at a hydrogen pressure of about 20 bar of hydrogen atmosphere.

In some embodiments, said compound of Formula XI is prepared by a process comprising reacting a compound of Formula X:

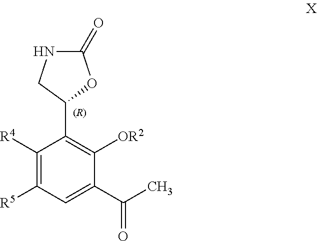

with R^{p}—NHNH₂, wherein R^{p}, R², R⁴, and R⁵ are as described above.

In some embodiments, R—NHNH₂ is t-butyl carbazate.

In some embodiments, said reacting of the compound of Formula X with R^{p}—NHNH₂ is performed in the presence of a catalytic amount of an acid.

In some embodiments, said acid is hydrochloric acid.

In some embodiments, about 0.01 to about 0.05 equivalents of hydrochloric acid is used based on 1 equivalent of the compound of Formula X.

In some embodiments, said reacting of the compound of Formula X with R^{p}—NHNH₂ is performed at about room temperature.

In some embodiments, said reacting of the compound of Formula X with R^{p}—NHNH₂ is performed in a solvent component comprising tetrahydrofuran.

In some embodiments, said compound of Formula X is prepared by a process comprising reacting a compound of Formula IX:

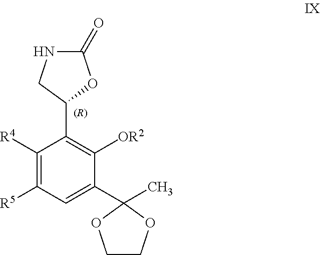

with an acid, wherein R², R⁴, and R⁵ are as described above.

In some embodiments, said acid is hydrochloric acid.

In some embodiments, said hydrochloric acid is 6 N aqueous hydrochloric acid.

In some embodiments, about 5 to about 6 equivalents of hydrochloric acid is used based on 1 equivalent of the compound of Formula IX.

In some embodiments, said reacting of the compound of Formula IX with an acid is performed in a solvent component comprising tetrahydrofuran.

In some embodiments, said reacting of the compound of Formula IX with an acid is performed at about room temperature.

In some embodiments, after the compound of Formula X is formed, a base is added to the reaction mixture to neutralize the acid.

In some embodiments, said compound of Formula IX is prepared by a process comprising reacting a compound of Formula VIII:

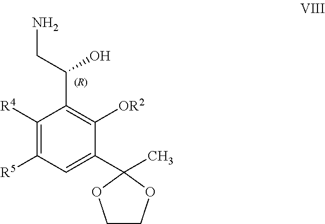

with carbonyldiimidazole, wherein R², R⁴, and R⁵ are as described above.

In some embodiments, about 2 to about 4 equivalents of carbonyldiimidazole are used based on 1 equivalent of the compound of Formula VIII.

In some embodiments, the compound of Formula VIII is reacted with said carbonyldiimidazole in portions, wherein 0.5 equivalents of carbonyldiimidazole is added to the reaction mixture at a time.

In some embodiments, said reacting of the compound of Formula VIII with carbonyldiimidazole is performed at a temperature of from about 50° C. to about 65° C.

In some embodiments, said reacting of the compound of Formula VIII with carbonyldiimidazole is performed in a solvent component comprising tetrahydrofuran.

In some embodiments, said compound of Formula VIII is prepared by a process comprising reacting a compound of Formula VII:

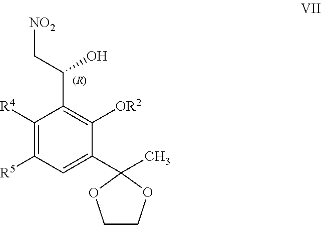

with hydrogen gas in the presence of one or more independently selected hydrogenation catalysts, wherein R², R⁴, and R⁵ are as described above.

In some embodiments, said hydrogenation catalyst comprises platinum.

In some embodiments, said hydrogenation catalyst is 1% Pt-2% V on activated carbon.

In some embodiments, about 30 wt % to about 50 wt % of hydrogenation catalyst is used based on the compound of Formula VII.

In some embodiments, said reacting of the compound of Formula VII with hydrogen gas is performed at about room temperature.

In some embodiments, said reacting of the compound of Formula VII with hydrogen gas is performed at a hydrogen pressure of about 50-60 psi of hydrogen atmosphere.

In some embodiments, said reacting of the compound of Formula VII with hydrogen gas is performed in the presence of an acid.

In some embodiments, said reacting of the compound of Formula VII with hydrogen gas is performed in the presence of acetic acid.

In some embodiments, about 1.1 to about 1.3 equivalents of acetic acid is used based on 1 equivalent of the compound of Formula VII.

In some embodiments, said reacting of the compound of Formula VII with hydrogen gas is performed in a solvent component comprising ethanol.

In some embodiments, said compound of Formula VIII is prepared by a process comprising reacting a compound of Formula VIII-rac

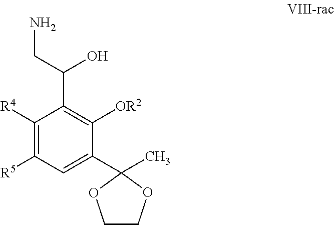

with an acidic chiral resolving agent, wherein R², R⁴, and R⁵ are as described above.

In some embodiments, the acidic chiral resolving agent is (S)-5-oxopyrrolidine-2-carboxylic acid (also known as 2S-pyroglutamic acid).

In some embodiments, about 0.5 equivalents of (S)-5-oxopyrrolidine-2-carboxylic acid is used based on 1 equivalent of the compound of Formula VIII-rac.

In some embodiments, said reacting of the compound of Formula VIII-rac with the acidic chiral resolving agent is performed at about room temperature.

In some embodiments, said reacting of the compound of Formula VIII-rac with the acidic chiral resolving agent is performed in a solvent component comprising ethanol and/or tetrahydrofuran.

In some embodiments:

(i) said compound of Formula VIII-rac is reacted with an acidic chiral resolving agent to form a protonated compound of Formula VIII; and

(ii) said protonated compound of Formula VIII is reacted with a base, thereby forming the compound of Formula VIII.

In some embodiments, said compound of Formula VIII-rac is prepared by a process comprising reacting a compound of Formula VII-rac:

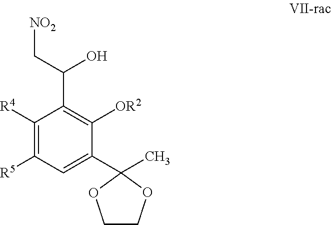

with hydrogen gas in the presence of one or more independently selected hydrogenation catalysts, wherein R², R⁴, and R⁵ are as described above.

In some embodiments, said hydrogenation catalyst comprises platinum.

In some embodiments, said hydrogenation catalyst is 1% Pt-2% V on activated carbon.

In some embodiments, about 30 wt % to about 50 wt % of hydrogenation catalyst is used based on the compound of Formula VII-rac.

In some embodiments, said reacting of the compound of Formula VII-rac with hydrogen gas is performed at about room temperature.

In some embodiments, said reacting of the compound of Formula VII-rac with hydrogen gas is performed at a hydrogen pressure of about 50-60 psi of hydrogen atmosphere.

In some embodiments, said reacting of the compound of Formula VII-rac with hydrogen gas is performed in the presence of an acid.

In some embodiments, said reacting of the compound of Formula VII-rac with hydrogen gas is performed in the presence of acetic acid.

In some embodiments, about 1.1 to about 1.3 equivalents of acetic acid is used based on 1 equivalent of the compound of Formula VII-rac.

In some embodiments, said reacting of the compound of Formula VII-rac with hydrogen gas is performed in a solvent component comprising ethanol.

In some embodiments, said compound of Formula VII is prepared by a process comprising reacting a compound of Formula VI:

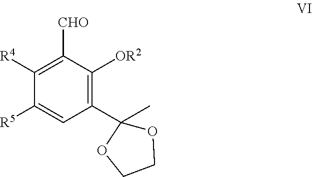

with nitromethane in the presence of a chiral catalyst, and an amine base, wherein R², R⁴, and R⁵ are as described above.

In some embodiments, the chiral catalyst is Cu[(−)-Sparteine]Cl₂.

In some embodiments, about 0.1 equivalent of Cu[(−)-Sparteine]Cl₂ is used based on 1 equivalent of the compound of Formula VI.

In some embodiments, about 7 to about 9 equivalents of nitromethane is used based on 1 equivalent of the compound of Formula VI.

In some embodiments, said amine base is triethylamine.

In some embodiments, about 0.01 to about 0.02 equivalents of amine base is used based on 1 equivalent of the compound of Formula VI.

In some embodiments, said reacting of the compound of Formula VI with nitromethane in the presence of a chiral catalyst, and an amine base is performed at a starting temperature of about 0° C. and is allowed to gradually warm to room temperature.

In some embodiments, said reacting is performed under an inert atmosphere.

In some embodiments, said reacting of the compound of Formula VI with nitromethane in the presence of a chiral catalyst, and an amine base is performed in a solvent component comprising methanol.

In some embodiments, said compound of Formula VII-rac is prepared by a process comprising reacting a compound of Formula VI:

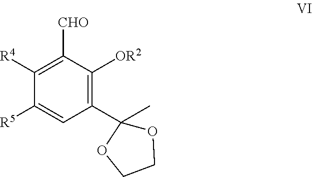

with nitromethane in the presence of a base, wherein R², R⁴, and R⁵ are as described above.

In some embodiments, about 3 to about 5 equivalents of nitromethane is used based on 1 equivalent of the compound of Formula VI.

In some embodiments, the base is sodium hydroxide.

In some embodiments, about 0.1 equivalents of sodium hydroxide is used based on 1 equivalent of the compound of Formula VI.

In some embodiments, said reacting of Formula VI with nitromethane in the presence of a base is performed in a solvent component comprising methanol.

In some embodiments, said reacting of Formula VI with nitromethane in the presence of a base is performed at room temperature.

In some embodiments, said compound of Formula VI is prepared by a process comprising reacting a compound of Formula V-a:

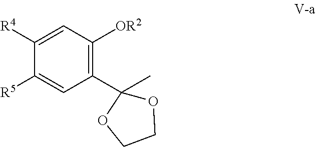

with N,N-dimethylformamide or N-formylmorpholine in the presence of lithium diisopropylamide, wherein R², R⁴, and R⁵ are as described above.

In some embodiments, said lithium diisopropylamide is prepared by reacting N,N-dimethylformamide or N-formylmorpholine in the presence of n-butyllithium.

In some embodiments, said lithium diisopropylamide is prepared at a temperature of from about −75° C. to about 5° C.

In some embodiments:

(i) said compound of Formula V-a is reacted with lithium diisopropylamide to form a first mixture; and

(ii) N,N-dimethylformamide or N-formylmorpholine is added to said first mixture to form a second mixture.

In some embodiments, the reaction takes place in the presence of about 1.2 to about 1.3 equivalents of amine base, based on 1 equivalent of the compound of Formula V-a.

In some embodiments, about 1.4 to about 1.6 equivalents of N,N-dimethylformamide or N-formylmorpholine is used based on 1 equivalent of the compound of Formula V-a.

In some embodiments, said compound of Formula V-a is prepared by a process comprising reacting a compound of Formula IV-a:

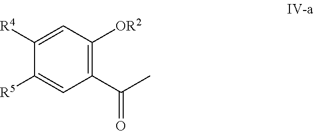

with 1,2-ethanediol in the presence of p-toluenesulfonic acid, wherein R², R⁴, and R⁵ are as described above.

In some embodiments, said p-toluenesulfonic acid is p-toluenesulfonic acid monohydrate.

In some embodiments, about 2.2 to about 2.7 equivalents of 1,2-ethanediol is used based on 1 equivalent of the compound of Formula IV-a.

In some embodiments, about 0.05 to about 0.1 equivalents of p-toluenesulfonic acid is used based on 1 equivalent of the compound of Formula IV-a.

In some embodiments, said reacting is performed at about reflux.

In some embodiments, said compound of Formula IV-a is prepared by a process comprising reacting a compound of Formula IIa:

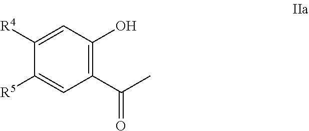

with R²—X¹ in the presence of an alkali metal carbonate base, wherein:

X¹ is halide; and

R², R⁴, and R⁵ are as described above.

In some embodiments, X¹ is iodide.

In some embodiments, said alkali metal carbonate base is potassium carbonate.

In some embodiments, about 1.1 to about 1.3 equivalents of R²—X¹ is used based on 1 equivalent of the compound of Formula IIa.

In some embodiments, about 1.8 to about 2.2 equivalents of alkali metal carbonate base is used based on 1 equivalent of the compound of Formula IIa.

In some embodiments, said reacting is performed at about 55° C. to about 65° C.

In some embodiments, said compound of Formula IIa is prepared according to procedures described in U.S. Publication No. 2013-0059835A1.

In some embodiments, said compound of Formula VI is prepared by a process comprising reacting a compound of Formula V-b:

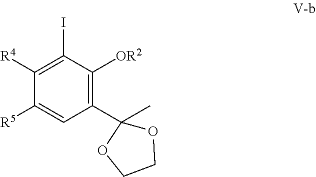

with a (C₁₋₄ alkyl)magnesium halide complex to form a first mixture, wherein R², R⁴, and R⁵ are as described above.

In some embodiments, said (C₁₋₄ alkyl)magnesium halide complex is 1.3 M isopropylmagnesium chloride lithium chloride complex.

In some embodiments, about 1.1 to about 1.3 equivalents of said (C₁₋₄ alkyl)magnesium halide complex is used based on 1 equivalent of the compound of Formula V-b.

In some embodiments, said reacting further comprises adding N-formylmorpholine to said first mixture to form a second mixture comprising the compound of Formula VI.

In some embodiments, about 1.8 to about 2.2 equivalents of N-formylmorpholine is used based on 1 equivalent of the compound of Formula V-b.

In some embodiments, said reacting is performed at a temperature of from about −5° C. to about 10° C.

In some embodiments, said compound of Formula V-b is prepared according to procedures described in U.S. Publication No. 2013-0059835A1.

In some embodiments, said compound of Formula V-a is prepared by a process comprising reacting a compound of Formula IV-c:

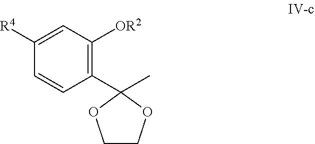

with a halogenating agent, a cyanating agent or an alkylating agent wherein R² and R⁴ are as described above.

In some embodiments, the compound of Formula IV-c is reacted with a halogenating agent.

In some embodiments, the halogenating agent is N-chlorosuccinamide.

In some embodiments, about 1.1 to about 1.3 equivalents of N-chlorosuccinamide is used based on 1 equivalent of the compound of Formula IV-c.

In some embodiments, said reacting is performed at about 15° C. to about 20° C.

In some embodiments, said reacting is performed using DMF as a reaction solvent.

In some embodiments, said compound of Formula IV-c is prepared by a process comprising reacting a compound of Formula III-c:

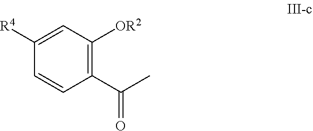

with 1,2-ethanediol in the presence of p-toluenesulfonic acid and triethyl orthoformate, wherein R² and R⁴ are as described above.

In some embodiments, said p-toluenesulfonic acid is p-toluenesulfonic acid monohydrate.

In some embodiments, about 2 to about 4 equivalents of 1,2-ethanediol is used based on 1 equivalent of the compound of Formula III-c.

In some embodiments, about 0.05 to about 0.1 equivalents of p-toluenesulfonic acid is used based on 1 equivalent of the compound of Formula III-c.

In some embodiments, about 2 to about 3 equivalents of triethyl orthoformate is used based on 1 equivalent of the compound of Formula III-c.

In some embodiments, said reacting is performed at about reflux.

In some embodiments:

(i) said compound of Formula III-c is reacted at about reflux with 1,2-ethanediol in the presence of p-toluenesulfonic acid to form a first mixture; and

(ii) said first mixture is cooled, triethyl orthoformate is added to said first mixture to form a second mixture and said second mixture is reacted at about reflux.

In some embodiments, said compound of Formula III-c is prepared by a process comprising reacting a compound of Formula II-c:

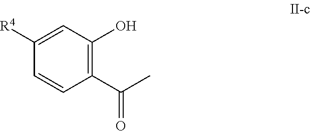

with R²—X¹ in the presence of an alkali metal carbonate base, wherein R² and R⁴ are as described above.

In some embodiments, said alkali metal carbonate base is potassium carbonate.

In some embodiments, about 1.1 to about 1.75 equivalents of R²—X¹ is used based on 1 equivalent of the compound of Formula II-c.

In some embodiments, about 1.5 to about 2.2 equivalents of alkali metal carbonate base is used based on 1 equivalent of the compound of Formula II-c.

In some embodiments, said reacting is performed at about 45° C. to about 65° C.

In some embodiments, said compound of Formula II-c is prepared according to procedures described in U.S. Publication No. 2013-0059835A1.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

The salts and compounds described herein can be asymmetric (e.g., having one or more stereocenters). If no stereochemistry is indicated, then all stereoisomers, such as enantiomers and diastereomers, are intended unless otherwise indicated by the structure or name. Salts and compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the salts and compounds described herein, and all such stable isomers are contemplated in the present disclosure. Cis and trans geometric isomers of the salts and compounds of the present disclosure are described and may be isolated as a mixture of isomers or as separated isomeric forms.

Resolution of racemic mixtures of salts and compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid, pyroglutamic acid or the various optically active camphorsulfonic acids such as 0-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Salts and compounds of the present disclosure can also include all isotopes of atoms occurring in the intermediates or final salts or compounds. Isotopes include those atoms having the same atomic number but different mass numbers. For example, isotopes of hydrogen include tritium and deuterium.

In some embodiments, the compounds or salts can be found together with other substances such as water and solvents (e.g. hydrates and solvates) or can be isolated.

In some embodiments, the compounds described herein, or salts thereof (e.g., the hydrochloric acid salt of the compound of Formula I), are substantially isolated. By “substantially isolated” is meant that the compound is at least partially or substantially separated from the environment in which it was formed or detected. Partial separation can include, for example, a composition enriched in the compounds of the present disclosure. Substantial separation can include compositions containing at least about 50%, at least about 60%, at least about 70%, at least about 80%, at least about 90%, at least about 95%, at least about 97%, or at least about 99% by weight of the compounds of the present disclosure, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As will be appreciated, the compounds provided herein, including salts thereof, can be prepared using known organic synthesis techniques and can be synthesized according to any of numerous possible synthetic routes. The processes described herein can be monitored according to any suitable method known in the art. For example, product formation can be monitored by spectroscopic means, such as nuclear magnetic resonance spectroscopy (e.g., 1H or 13C), infrared spectroscopy, or spectrophotometry (e.g., UV-visible); or by chromatography such as high performance liquid chromatography (HPLC) or thin layer chromatography (TLC) or other related techniques.

As used herein, the term “reacting” is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Suitable solvents can include halogenated solvents such as carbon tetrachloride, bromodichloromethane, dibromochloromethane, bromoform, chloroform, bromochloromethane, dibromomethane, butyl chloride, dichloromethane, tetrachloroethylene, trichloroethylene, 1,1,1-trichloroethane, 1,1,2-trichloroethane, 1,1-dichloroethane, 2-chloropropane, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable ether solvents include: dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, t-butyl methyl ether, mixtures thereof and the like.

Suitable protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol.

Suitable aprotic solvents can include, by way of example and without limitation, tetrahydrofuran (THF), N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

The reactions of the processes described herein can be carried out at appropriate temperatures which can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

As used herein, “about” when referring to a measurable value such as an amount, a temperature, a temporal duration, a dosage and the like, is meant to encompass variations of 10%. In certain embodiments, “about” can include variations of 5%, +1%, or ±0.1% from the specified value and any variations there between, as such variations are appropriate to perform the disclosed methods and processes.

The reactions of the processes described herein can be carried out in air or under an inert atmosphere. Typically, reactions containing reagents or products that are substantially reactive with air can be carried out using air-sensitive synthetic techniques that are well known to the skilled artisan.

### Methods of Use

The salts and compounds of the present disclosure can modulate activity of one or more of various kinases including, for example, phosphoinositide 3-kinases (PI3Ks). The term “modulate” is meant to refer to an ability to increase or decrease the activity of one or more members of the PI3K family. Accordingly, the salts and compounds of the present disclosure can be used in methods of modulating a PI3K by contacting the PI3K with any one or more of the salts, compounds or compositions described herein. In some embodiments, salts and compounds of the present disclosure can act as inhibitors of one or more PI3Ks. In further embodiments, the salts and compounds of the present disclosure can be used to modulate activity of a PI3K in an individual in need of modulation of the receptor by administering a modulating amount of a compound of the present disclosure. In some embodiments, modulating is inhibiting.

Given that cancer cell growth and survival is impacted by multiple signaling pathways, the present disclosure is useful for treating disease states characterized by drug resistant kinase mutants. In addition, different kinase inhibitors, exhibiting different preferences in the kinases which they modulate the activities of, may be used in combination. This approach could prove highly efficient in treating disease states by targeting multiple signaling pathways, reduce the likelihood of drug-resistance arising in a cell, and reduce the toxicity of treatments for disease.

Kinases to which the present salts and compounds bind and/or modulate (e.g., inhibit) include any member of the PI3K family. In some embodiments, the PI3K is PI3Kα, PI3Kβ, PI3Kδ, or PI3Kγ. In some embodiments, the PI3K is PI3Kδ or PI3Kγ. In some embodiments, the PI3K is PI3Kδ. In some embodiments, the PI3K is PI3Kγ. In some embodiments, the PI3K includes a mutation. A mutation can be a replacement of one amino acid for another, or a deletion of one or more amino acids. In such embodiments, the mutation can be present in the kinase domain of the PI3K.

In some embodiments, more than one salt or compound of the present disclosure is used to inhibit the activity of one kinase (e.g., PI3Kδ or PI3Kγ).

In some embodiments, more than one salt or compound of the present disclosure is used to inhibit more than one kinase, such as at least two kinases (e.g., PI3Kδ and PI3Kγ).

In some embodiments, one or more of the salts or compounds is used in combination with another kinase inhibitor to inhibit the activity of one kinase (e.g., PI3Kδ or PI3Kγ).

In some embodiments, one or more of the salts or compounds is used in combination with another kinase inhibitor to inhibit the activities of more than one kinase (e.g., PI3Kδ or PI3Kγ), such as at least two kinases.

The salts and compounds of the present disclosure can be selective. By “selective” is meant that the compound binds to or inhibits a kinase with greater affinity or potency, respectively, compared to at least one other kinase. In some embodiments, the salts and compounds of the present disclosure are selective inhibitors of PI3Kδ or PI3Kγ over PI3Kα and/or PI3Kβ. In some embodiments, the salts and compounds of the present disclosure are selective inhibitors of PI3Kδ (e.g., over PI3Kα, PI3Kβ and PI3Kγ).

In some embodiments, the salts and compounds of the present disclosure are selective inhibitors of PI3Kδ (e.g., over PI3Kα, PI3Kβ and PI3Kγ). In some embodiments, selectivity can be at least about 2-fold, 5-fold, 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the Kₘ ATP concentration of each enzyme. In some embodiments, the selectivity of salts and compounds of the present disclosure can be determined by cellular assays associated with particular PI3K kinase activity.

Another aspect of the present disclosure pertains to methods of treating a kinase (such as PI3K)-associated disease or disorder in an individual (e.g., patient) by administering to the individual in need of such treatment a therapeutically effective amount or dose of one or more salts or compounds of the present disclosure or a pharmaceutical composition thereof. A PI3K-associated disease can include any disease, disorder or condition that is directly or indirectly linked to expression or activity of the PI3K, including overexpression and/or abnormal activity levels. In some embodiments, the disease can be linked to Akt (protein kinase B), mammalian target of rapamycin (mTOR), or phosphoinositide-dependent kinase 1 (PDK1). In some embodiments, the disease can be inflammation, atherosclerosis, psoriasis, restenosis, benign prostatic hypertrophy, bone disorders, pancreatitis, angiogenesis, diabetic retinopathy, arthritis, immunological disorders, kidney disease, or cancer. A PI3K-associated disease can also include any disease, disorder or condition that can be prevented, ameliorated, or cured by modulating PI3K activity. In some embodiments, the disease is characterized by the abnormal activity of PI3K. In some embodiments, the disease is characterized by mutant PI3K. In such embodiments, the mutation can be present in the kinase domain of the PI3K.

Examples of PI3K-associated diseases include immune-based diseases involving the system including, for example, rheumatoid arthritis, allergy, asthma, glomerulonephritis, lupus, or inflammation related to any of the above.

Further examples of PI3K-associated diseases include osteoarthritis, inflammatory bowel disease, myasthenia gravis, multiple sclerosis, or Sjögren's syndrome, and the like.

In some embodiments, the disease is selected from idiopathic thrombocytopenic purpura (ITP), vasculitis, systemic lupus erythematosus, lupus nephritis, pemphigus, autoimmune hemolytic anemia (AIHA), membranous nephropathy, chronic lymphocytic leukemia (CLL), Non-Hodgkin lymphoma (NHL), hairy cell leukemia, Mantle cell lymphoma, Burkitt lymphoma, small lymphocytic lymphoma, follicular lymphoma, lymphoplasmacytic lymphoma, extranodal marginal zone lymphoma, Hodgkin's lymphoma, Waldenstrom's macroglobulinemia, prolymphocytic leukemia, acute lymphoblastic leukemia, myelofibrosis, mucosa-associated lymphatic tissue (MALT) lymphoma, B-cell lymphoma, mediastinal (thymic) large B-cell lymphoma, lymphomatoid granulomatosis, splenic marginal zone lymphoma, primary effusion lymphoma, intravascular large B-cell lymphoma, plasma cell leukemia, extramedullary plasmacytoma, smouldering myeloma (aka asymptomatic myeloma), monoclonal gammopathy of undetermined significance (MGUS) and B cell lymphoma.

In some embodiments, the method is a method of treating idiopathic thrombocytopenic purpura (ITP) selected from relapsed ITP and refractory ITP.

In some embodiments, the method is a method of treating vasculitis selected from Behget's disease, Cogan's syndrome, giant cell arteritis, polymyalgia rheumatica (PMR), Takayasu's arteritis, Buerger's disease (thromboangiitis obliterans), central nervous system vasculitis, Kawasaki disease, polyarteritis nodosa, Churg-Strauss syndrome, mixed cryoglobulinemia vasculitis (essential or hepatitis C virus (HCV)-induced), Henoch-Schonlein purpura (HSP), hypersensitivity vasculitis, microscopic polyangiitis, Wegener's granulomatosis, and anti-neutrophil cytoplasm antibody associated (ANCA) systemic vasculitis (AASV).

In some embodiments, the method is a method of treating non-Hodgkin lymphoma (NHL) selected from relapsed NHL, refractory NHL, and recurrent follicular NHL.

In some embodiments, the method is a method of treating B cell lymphoma, wherein said B cell lymphoma is diffuse large B-cell lymphoma (DLBCL).

In some embodiments, the method is a method of treating B cell lymphoma, wherein said B cell lymphoma is activated B-cell like (ABC) diffuse large B cell lymphoma, or germinal center B cell (GCB) diffuse large B cell lymphoma.

In some embodiments, said lupus is systemic lupus erythematosus or lupus nephritis.

In some embodiments, said disease is breast cancer, prostate cancer, colon cancer, endometrial cancer, brain cancer, bladder cancer, skin cancer, cancer of the uterus, cancer of the ovary, lung cancer, pancreatic cancer, renal cancer, gastric cancer, or a hematological cancer.

In some embodiments, said hematological cancer is acute myeloblastic leukemia or chronic myeloid leukemia.

In some embodiments, said hematological cancer is lymphoid malignancies of B-cell origin including, indolent/aggressive B-cell non-Hodgkin's lymphoma (NHL), and Hodgkin's lymphoma (HL).

In some embodiments, said disease is acute lung injury (ALI) or adult respiratory distress syndrome (ARDS).

As used herein, the term “contacting” refers to the bringing together of indicated moieties in an in vitro system or an in vivo system. For example, “contacting” a PI3K with a compound of the present disclosure includes the administration of a compound of the present disclosure to an individual or patient, such as a human, having a PI3K, as well as, for example, introducing a compound of the present disclosure into a sample containing a cellular or purified preparation containing the PI3K.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. In some embodiments, the dosage of the compound administered to a patient or individual is about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Combination Therapies One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds and salts described herein for treatment of PI3K-associated diseases, disorders or conditions, or diseases or conditions as described herein. The agents can be combined with the present compounds and salts in a single dosage form, or the agents can be administered simultaneously or sequentially as separate dosage forms.

Compounds and salts described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFOR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the PI3K inhibitor as described herein can be combined with inhibitors of kinases associated with the PIK3/Akt/mTOR signaling pathway, such as Akt (including Akt1, Akt2 and Akt3) and mTOR kinases as well as additional PI3K inhibitors.

In some embodiments, compounds and salts described herein can be used in combination with one or more inhibitors of the enzyme or protein receptors such as HPK1, SBLB, TUT4, A2A/A2B, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds and salts described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds and salts described herein can be used in combination with targeted therapies, including JAK kinase inhibitors (e.g., ruxolitinib, additional JAK1/2 and JAK1-selective inhibitors such as baricitinib, INCB39110, or INCB54707), Pim kinase inhibitors (e.g., LGH447, INCB053914 and SGI-1776), PI3 kinase inhibitors including PI3K-delta selective and broad spectrum PI3K inhibitors (e.g., INCB50465 (i.e., parsaclisib)), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), FGFR inhibitors, PARP inhibiors (e.g., olaparib or rucaparib), and inhibitors of BTK such as ibrutinib.

For treating cancer and other proliferative diseases, compounds and salts described herein can be used in combination with chemotherapeutic agents, agonists or antagonists of nuclear receptors, or other anti-proliferative agents. Compounds and salts described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds and salts described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1B), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds and salts provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20170362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is MGA012 (i.e., retifanlimab), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is MGA012 (i.e., retifanlimab). In some embodiments, the anti-PD1 antibody is SHR-1210. Other anti-cancer agent(s) include antibody therapeutics such as 4-1BB (e.g. urelumab, utomilumab).

In some embodiments, the compounds and salts of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds and salts of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds and salts of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds and salts described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds and salts of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds and salts described herein for the treatment of cancer include chemotherapeutic agents, targeted cancer therapies, immunotherapies or radiation therapy. Compounds and salts described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds and salts described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds and salts described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with PI3K inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with PI3K inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds and salts described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with PI3K inhibitors. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds and salts described herein. In some embodiments, the JAK inhibitor is selective for JAK1 over JAK2 and JAK3.

Other suitable agents for use in combination with compounds and salts described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds and salts described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds and salts described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds and salts described herein may also be combined with immunotherapy drugs, including cytokines such as interferon alpha, interleukin 2, and tumor necrosis factor (TNF) in.

Suitable chemotherapeutic or other anti-cancer agents include, for example, anti-metabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds and salts of the present disclosure can be used in combination with bone marrow transplant for the treatment of a variety of tumors of hematopoietic origin.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

As provided throughout, the additional compounds, inhibitors, agents, etc. can be combined with the present compounds and salts in a single or continuous dosage form, or they can be administered simultaneously or sequentially as separate dosage forms.

### Pharmaceutical Formulations and Dosage Forms

When employed as pharmaceuticals, compounds and salts described herein can be administered in the form of pharmaceutical compositions which refers to a combination of one or more compounds and salts described herein, and at least one pharmaceutically acceptable carrier or excipient. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more compounds and salts described herein in combination with one or more pharmaceutically acceptable carriers or excipients. In making the compositions described herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is suitable for topical administration.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds and salts of the disclosure may be milled using known milling procedures such as wet milling to obtain a particle size appropriate for tablet formation and for other formulation types. Finely divided (nanoparticulate) preparations of the compounds and salts of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions described herein can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from, for example, about 5 mg to about 1000 mg, about 5 mg to about 100 mg, about 100 mg to about 500 mg or about 10 to about 30 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound or salt can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The therapeutic dosage of a compound or salt of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds and salts of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid pre-formulation composition containing a homogeneous mixture of one or more compounds or salts described herein. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds, salts, or compositions as described herein can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of the compounds and salts in a pharmaceutical composition can vary depending upon a number of factors including dosage, chemical characteristics (e.g., hydrophobicity), and the route of administration. For example, compounds and salts of the present disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Compounds and salts described herein can also be formulated in combination with one or more additional active ingredients, which can include any pharmaceutical agent such as anti-viral agents, vaccines, antibodies, immune enhancers, immune suppressants, anti-inflammatory agents and the like.

### Kits

The present disclosure also includes pharmaceutical kits useful, for example, in the treatment or prevention of PI3K-associated diseases or disorders, such as cancer, which include one or more containers containing a pharmaceutical composition comprising a therapeutically effective amount of a compound or salt of the present disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The salts of the compound of Formula I and the compound of Formula I have been found to be PI3K inhibitors according to at least one assay described herein.

### EXAMPLES

The invention will be described in greater detail by way of specific examples. The following examples are offered for illustrative purposes, and are not intended to limit the invention in any manner. The example compounds, or salts thereof, containing one or more chiral centers were obtained in racemate form or as isomeric mixtures, unless otherwise specified.

### General Methods

Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature [see “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with the 30×100 mm column was 60 mL/minute.

pH=10 purifications: Waters XBridge C₁₈ 5 μm, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature [See “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with 30×100 mm column was 60 mL/minute.

Some of the compounds prepared were also analyzed via Differential Scanning Calorimetry (DSC). Typical DSC instrument conditions are as follows:

TA Instrument Differential Scanning Calorimetry, Model Q200 with autosampler: 30-350° C. at 10° C./min; T-zero aluminum sample pan and lid; nitrogen gas flow at 50 mL/min.

Mettler Toledo Differential Scanning Calorimetry (DSC) 822 Instrument: 40-340° C. at a heating rate of 10° C./min.

Some of the compounds prepared were also analyzed via Thermogravimetric Analysis (TGA). Typical TGA instrument conditions are as follows:

TA Instrument Thermogravimetric Analyzer, Model Pyris: Temperature ramp from 25° C. to 300° C. at 10° C./min; nitrogen purge gas flow at 60 mL/min; TGA ceramic crucible sample holder.

TA Instrument Q500: Temperature ramp from 20° C. to 300° C. at 10° C./min.

Some of the compounds prepared were also analyzed via X-Ray Powder Diffraction (XRPD). Typical XRPD instrument conditions are as follows:

Bruker D2 PHASER X-Ray Powder Diffractometer instrument: X-ray radiation wavelength: 1.05406 Å CuKAI; x-ray power: 30 KV, 10 mA; sample powder: dispersed on a zero-background sample holder; general measurement conditions: start Angle—5 degree, Stop Angle—60 degree, Sampling—0.015 degree, Scan speed—2 degree/min.

Rigaku Miniflex Powder Diffractometer: Cu at 1.054056 Å with Kβ filter; general measurement conditions: start Angle—3 degree, Stop Angle—45 degree, Sampling—0.02 degree, Scan speed—2 degree/min.

### Example 1. Synthesis of 3-chloro-6-ethoxy-2-fluoro-5-(2-methyl-1,3-dioxolan-2-yl)benzaldehyde

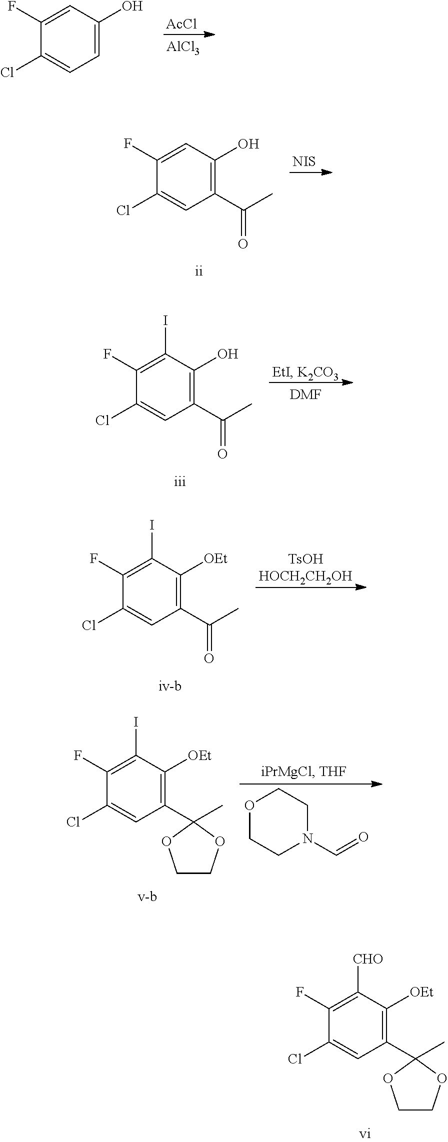

### Step 1. 1-(5-Chloro-4-fluoro-2-hydroxyphenyl)ethanone (ii)

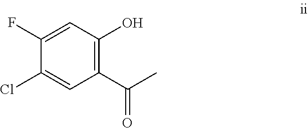

4-Chloro-3-fluorophenol (i, 166 g, 1.11 mol) and acetyl chloride (107 mL, 1.50 mol) were charged to a 5-L flask at room temperature. The reaction mixture was stirred and turned to a clear solution while the batch temperature was recorded to decrease to 6° C. The reaction mixture was then heated to 60° C. for 2 h. The reaction mixture was charged with nitrobenzene (187.5 mL, 1.82 mol) and subsequently cooled to room temperature. Aluminum trichloride (160 g, 1.2 mmol) was then added to the mixture in three portions (50 g, 50 g, and 60 g at 5 min intervals). The batch temperature increased to 78° C. upon completion of addition. The reaction mixture was then heated at 100-120° C. for 3 h, at which time HPLC analysis showed the reaction was complete. The reaction mixture was then cooled to 0° C. and charged with hexanes (0.45 L), ethyl acetate (0.55 L), and then charged slowly with 1.0 N aqueous hydrochloric acid (1.0 L) at room temperature. The addition of aqueous hydrochloride acid was exothermic and the batch temperature increased from 26° C. to 60° C. The resulting mixture was stirred at room temperature for 20 min. The layers were separated and the organic layer was washed sequentially with 1.0 N aqueous hydrochloric acid (2×600 mL) and water (400 mL). The organic layer was then extracted with 1.0 N aqueous sodium hydroxide solution (2×1.4 L). The combined basic solutions were acidified to pH 2 by addition of 12 N aqueous hydrochloric acid until no further precipitate was separated. The resulting solid was collected by filtration, washed with water and dried in the filter funnel under suction to give compound ii as a yellow solid (187.4 g, 89.5%). ¹H NMR (400 MHz, CDCl₃) δ 12.44 (d, J=1.4 Hz, 1H), 7.78 (d, J=8.1 Hz, 1H), 6.77 (d, J=10.2 Hz, 1H), 2.61 (s, 3H).

### Step 2. 1-(5-Chloro-4-fluoro-2-hydroxy-3-iodophenyl)ethanone (iii)

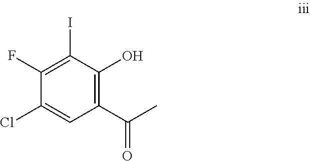

1-(5-Chloro-4-fluoro-2-hydroxyphenyl)ethanone (ii, 100.0 g, 530.3 mmol) was dissolved in acetic acid (302 mL) and N-iodosuccinimide (179.2 g, 796.5 mmol) was added to the solution. The reaction mixture was stirred at from about 61° C. to about 71° C. for 2 h, at which time HPLC analysis indicated that the reaction was complete. The reaction mixture was then cooled to room temperature, water (613 mL) was added, and the resulting slurry was stirred at room temperature for 30 min. The product was collected by filtration and rinsed with water to afford brown solids. The wet product was dissolved in acetic acid (400 mL) at 60° C. Water (800 mL) was added (over 15 min) to the solution to precipitate pure product. The product was collected by filtration and washed with water (100 mL). The product was dried on the filter funnel under suction for 18 h to give compound iii as a brown solid (164.8 g, 95.0% yield). ¹H NMR (300 MHz, DMSO-d₆) δ13.34 (s, 1H), 8.26 (d, J=8.4 Hz, 1H), 2.68 (s, 3H).

### Step 3. 1-(5-Chloro-2-ethoxy-4-fluoro-3-iodophenyl)ethanone (iv-b)

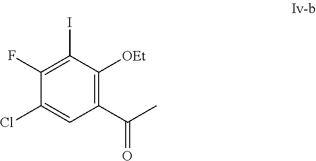

In a 5-L three-necked round bottom flask equipped with a condenser and a thermometer, 1-(5-chloro-4-fluoro-2-hydroxy-3-iodophenyl)ethanone (iii, 280 g, 840 mmol) was dissolved in N,N-dimethylformamide (600 mL). During the dissolution, the internal temperature dropped from 19.3° C. to 17.0° C. Iodoethane (81.2 mL, 1020 mmol) was added to the resulting mixture. Potassium carbonate (234 g, 1690 mmol) was then added over 2 min to the reaction mixture and no change in the batch temperature was observed. The reaction mixture was heated to 60° C. for 3 h, at which time HPLC analysis indicated the reaction was complete. The reaction mixture was allowed to cool to room temperature and the product was collected by filtration. The solids were dissolved in a mixture of DCM (1.0 L), hexane (500 ml), and water (2.1 L). The biphasic system was stirred at 20° C. for 20 min. The layers were separated and the aqueous layer was extracted with DCM (1.0 L). The combined organic layer was washed with water (2×250 mL) and brine (60 mL). The organic phase was separated, dried over anhydrous Na₂SO₄, filtered, and concentrated in vacuo to dryness to give compound iv as a yellow solid (292 g, 94% yield). ¹H NMR (400 MHz, DMSO-d₆) δ7.69 (d, J=8.4 Hz, 1H), 3.95 (q, J=7.0 Hz, 2H), 2.62 (s, 3H), 1.49 (t, J=7.0 Hz, 3H). LCMS for C₁₀H₁₀ClFIO₂ (M+H)⁺: m/z=342.9.

### Step 4. 2-(5-chloro-2-ethoxy-4-fluoro-3-iodophenyl)-2-methyl-1,3-dioxolane (v-b)

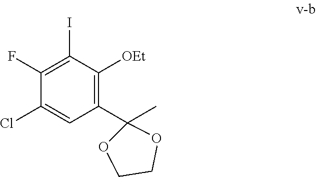 [SMILES: CCOc1c(C2(C)OCCO2)cc(Cl)c(F)c1I]

A solution of 1-(5-chloro-2-ethoxy-4-fluoro-3-iodophenyl)ethanone (iv-b, 250.0 g, 693.4 mmol) and 1,2-ethanediol (58.0 mL, 1040 mmol) in toluene (1.5 L) was treated with p-toluenesulfonic acid monohydrate (10.6 g, 55.5 mmol). The reaction flask was fitted with a Dean-Stark trap and the mixture was heated at reflux for 7 h. LCMS analysis indicated that the reaction mixture contained 8.3% starting material and 91.7% product. The reaction mixture was cooled to 106° C., and additional amount of 1,2-ethanediol (11.6 mL, 208 mmol) was introduced via syringe. The reaction mixture was then heated at reflux for an additional 8 h. LCMS analysis indicated that the reaction mixture contained 3.6% starting material and 96.4% product. The reaction mixture was cooled to 106° C., and additional 1,2-ethanediol (7.73 mL, 139 mmol) was introduced via syringe. The reaction mixture was heated under reflux for additional 15.5 h. LCMS analysis indicated that the reaction mixture contained 2.2% starting material and 97.8% product.

The reaction mixture was then cooled to 0° C. and water (200 ml) and aqueous saturated NaHCO₃ (300 ml) were added to adjust the mixture to a pH of 9. DCM (200 ml) was added and the batch was stirred for 10 min. The layers were separated and the aqueous layer was extracted with toluene (300 mL). The combined organic layer was washed sequentially with a mixture of water (200 ml) and aqueous saturated NaHCO₃ (200 ml), water (300 ml), brine (300 ml), dried over anhydrous Na₂SO₄, filtered, and concentrated in vacuo to dryness to provide the crude compound v as light brown solid (268 g 100% yield). ¹H NMR (400 MHz, CDCl₃) δ 7.59 (d, J=8.6 Hz, 1H), 4.26-3.96 (m, 4H), 3.92-3.72 (m, 2H), 1.74 (s, 3H), 1.50 (t, J=7.0 Hz, 3H). LCMS for C₁₂H₁₄ClFIO₃ (M+H)+: m/z=387.0.

### Step 5. 3-chloro-6-ethoxy-2-fluoro-5-(2-methyl-1,3-dioxolan-2-yl)benzaldehyde (vi)

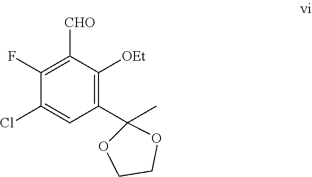 [SMILES: CCOc1c(C2(C)OCCO2)cc(Cl)c(F)c1C=O]

To a stirred solution of 2-(5-chloro-2-ethoxy-4-fluoro-3-iodophenyl)-2-methyl-1,3-dioxolane (v, 135.0 g, 349.2 mmol) (86.8% purity by HPLC with 5.5% of the ketone) in anhydrous tetrahydrofuran (300 mL) at about 0° C. to about 3° C. was slowly added 1.3 M isopropylmagnesium chloride lithium chloride complex in THE (322.3 mL, 419.0 mmol) over 1 h. The reaction mixture was stirred at from about 0° C. to about 5° C. for 30 min. at which time LCMS analysis indicated the iodo-magnesium exchange reaction was complete. N-Formylmorpholine (71.1 mL, 700 mmol) was then added to the reaction mixture over 1 h at from about 0° C. to about 8° C. The reaction mixture was stirred at from about 0° C. to about 8° C. for an additional 1 h, at which time LCMS and HPLC analyses showed the starting material was consumed and a significant amount of de-iodination by-product, 2-(5-chloro-2-ethoxy-4-fluorophenyl)-2-methyl-1,3-dioxolane was observed. The reaction was quenched with an aqueous solution of citric acid (120.8 g, 628.6 mmol) in water (1.20 L) at 0° C. The quenched reaction mixture was then extracted with EtOAc (2×600 mL). The phases were readily separated. The combined organic layer was washed sequentially with water (300 ml) and brine (500 mL), dried over anhydrous Na₂SO₄, filtered, and concentrated in vacuo. The residue was purified by flash column chromatography on silica gel with 0-10% EtOAc/hexane to give the crude product compound vi as a pale yellow solid, which was a mixture of the desired product, 3-chloro-6-ethoxy-2-fluoro-5-(2-methyl-1,3-dioxolan-2-yl)benzaldehyde (vi, 80 g, 80%) containing 36 mol % of the de-iodination by-product, 2-(5-chloro-2-ethoxy-4-fluorophenyl)-2-methyl-1,3-dioxolane as indicated by NMR analysis. The crude product compound vi was further purified by formation of the corresponding sodium bisulfite adduct.

Sodium bisulfite (36.91 g, 354.7 mmol) was dissolved in water (74.3 mL, 4121 mmol). To a stirred solution of crude 3-chloro-6-ethoxy-2-fluoro-5-(2-methyl-1,3-dioxolan-2-yl)benzaldehyde (vi, 80.00 g, 177.3 mmol) in ethyl acetate (256.0 mL), the freshly prepared sodium bisulfite solution was added in one portion. The solution was stirred for about 10 min and precipitates were observed. The slurry was then stirred for an additional 1 h. The aldehyde-bisulfite adduct was collected by filtration, washed with EtOAc and dried under vacuum and nitrogen atmosphere for 20 h to give a white solid (58.2 g, 83.6% yield). To the aldehyde-bisulfite adduct (58.2 g, 148 mmol) mixed in 1.0 M aqueous sodium hydroxide (296 mL, 296 mmol) was added methyl t-butyl ether (600 mL) (MTBE). The reaction mixture was stirred at room temperature for 6 min to give a clear biphasic mixture, and stirring was continued for an additional 5 min. The organic phase was collected and the aqueous layer was extracted with MTBE (2×300 mL). The combined organic layers were washed with brine (300 mL), dried over anhydrous Na₂SO₄, filtered, and concentrated in vacuo to give pure compound vi as a white crystalline solid (31.4 g, 73.4% yield). ¹H NMR (400 MHz, CDCl₃) δ 10.27 (s, 1H), 7.78 (d, J=8.5 Hz, 1H), 4.10-3.96 (m, 4H), 3.87-3.76 (m, 2H), 1.72 (s, 3H), 1.44 (t, J=7.0 Hz, 3H). LCMS for C₁₃H₁₅ClFO₄ (M+H)⁺: m/z=289.0.

### Example 2. Alternative Synthesis of 3-chloro-6-ethoxy-2-fluoro-5-(2-methyl-1,3-dioxolan-2-yl)benzaldehyde

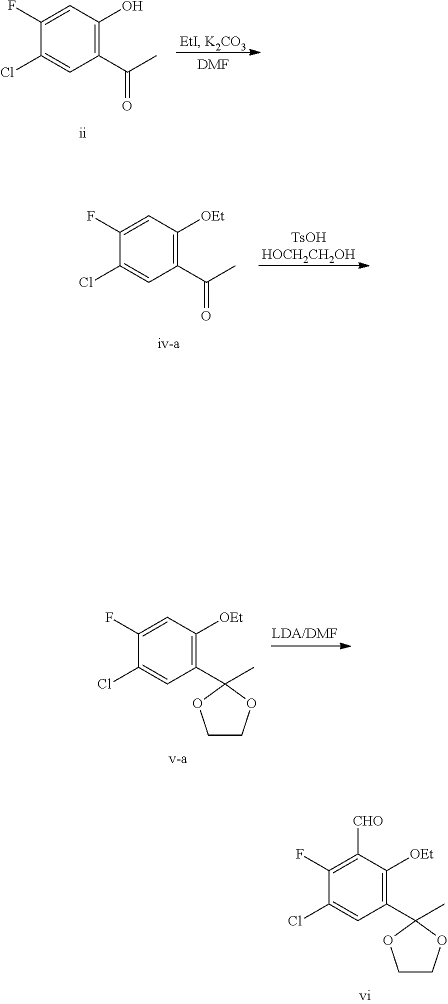

### Step 1. 1-(5-chloro-2-ethoxy-4-fluorophenyl)ethanone (iv-a)

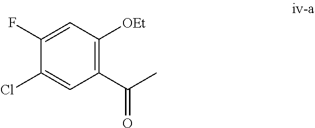

1-(5-Chloro-4-fluoro-2-hydroxyphenyl)ethanone (Compound ii from Example 1, Step 1, 1350 g, 7160 mmol), N,N-dimethylformamide (3.32 L), iodoethane (1340 g, 8590 mmol), and potassium carbonate (1980 g, 14300 mmol) were mixed together and stirred at room temperature for 45 min. The batch temperature went up to 55° C. from 22° C. The reaction mixture was heated to 60° C. for 1 h (the batch temperature reached 67° C. in 30 min and then dropped to 60° C.). HPLC analysis indicated all starting material was consumed. Water (10 L) was added in one portion (agitation will cease if water is added in portions). The resulting slurry was stirred at room temperature for 30 min. The product was collected by filtration and was rinsed with water (3 L). The product dried on the filter under vacuum for 5 days to give compound iv-a as a tan solid (1418 g). ¹H NMR (400 MHz, DMSO-d₆) δ 7.69 (d, J=8.9 Hz, 1H), 7.30 (d, J=11.6 Hz, 1H), 4.15 (q, J=7.0 Hz, 2H), 2.51 (s, 3H), 1.37 (t, J=7.0 Hz, 3H).

### Step 2. 2-(5-chloro-2-ethoxy-4-fluorophenyl)-2-methyl-1,3-dioxolane (v-a)

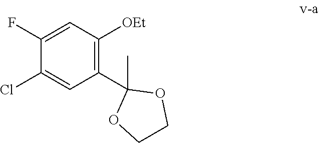 [SMILES: CCOc1cc(F)c(Cl)cc1C1(C)OCCO1]

A solution of 1-(5-chloro-2-ethoxy-4-fluorophenyl)ethanone (iv-a, 1481.0 g, 6836.3 mmol) was dissolved in toluene (6 L). 1,2-Ethanediol (953 mL, 17100 mmol) and p-toluenesulfonic acid monohydrate (104 g, 547 mmol) were added to the solution. The reaction mixture was heated to reflux at 104-110° C. with the use of a Dean-Stark trap to remove the water for 17.4 h. HPLC analysis indicated 37% of starting material remained unreacted. About 600 mL of distillate was removed and the reaction mixture was heated under reflux for additional 5 h (total 22 h). HPLC analysis indicated no further reaction.

It was speculated that residual K₂CO₃ in the starting material compound iv-a may have halted the reaction. Therefore, the reaction mixture was cooled to room temperature and washed with 1 N aqueous hydrochloric acid (3×6.66 L). After the aqueous acid wash, the organic layer was transferred back to the reaction vessel. 1,2-Ethanediol (381 mL, 6840 mmol) and p-toluenesulfonic acid monohydrate (104 g, 547 mmol) were added and the reaction mixture was heated under reflux for 16 h. HPLC analysis indicated about 20% of starting material remained unreacted. About 100 mL of distillate was removed. 1,2-Ethanediol (380 mL, 6800 mmol) was added and refluxed for 6 h (22 h total). HPLC indicated 7% of starting material remained unreacted. About 125 mL of distillate was removed. The reaction mixture was heated to reflux for 6 h (total 28 h). HPLC indicated 5.4% of starting material remained unreacted. About 125 mL of distillate was removed. The reaction mixture was heated to reflux for additional 7 h. HPLC analysis indicated 3.5% of starting material remained unreacted. About 80 mL of distillate was removed. The reaction was deemed complete at this point.

The reaction mixture was washed with a 1 N aqueous sodium hydroxide solution (2×5.5 L). The first basic wash was extracted with toluene (2.1 L). The combined toluene solution was washed with water (7 L) and concentrated to give 2153 g of dark oil. HPLC analysis indicated product purity at 93.8% with 1.90% of starting material and 0.79% of de-iodo product. ¹H NMR analysis indicated about 0.5 equivalent of toluene (about 256 g) remained in the product. The corrected yield of compound v-a was 88.0%. ¹H NMR (300 MHz, CDCl₃) δ 7.51 (d, J=8.8 Hz, 1H), 6.70 (d, J=11.0 Hz, 1H), 4.17-3.92 (m, 4H), 3.91-3.80 (m, 2H), 1.75 (s, 3H), 1.46 (t, J=7.0 Hz, 3H).

### Step 3. 3-Chloro-6-ethoxy-2-fluoro-5-(2-methyl-1,3-dioxolan-2-yl)benzaldehyde (vi)

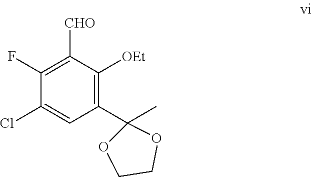 [SMILES: CCOc1c(C2(C)OCCO2)cc(Cl)c(F)c1C=O]

Into an oven-dried 3-L 4-neck round bottom flask equipped with an overhead stirrer, a 500 mL addition funnel, nitrogen inlet, septa, and thermocouple was charged N,N-diisopropylamine (87.8 mL, 626 mmol) and anhydrous tetrahydrofuran (1090 mL, 13500 mmol). This solution was cooled to −72° C. and charged with 2.5 M n-butyllithium in hexanes (261 mL, 652 mmol). The n-butyllithium solution was added over 18 min. The maximum internal temperature during the addition was −65°. The dry ice-acetone bath was replaced with an ice-water bath and the reaction mixture was warmed to about −5° C. to about 0° C. and held for 15 min. The reaction mixture was then cooled to −74.5° C.

To a separate 1-L round bottom flask containing 2-(5-chloro-2-ethoxy-4-fluorophenyl)-2-methyl-1,3-dioxolane (v-a, 136.1 g, 522.1 mmol) was added anhydrous tetrahydrofuran (456 mL) to dissolve the solids. The resulting solution was cooled in an ice bath to about 0° C. The solution containing compound v-a was transferred to the LDA solution over 40 minutes via a canula while maintaining the reaction temperature between −70° C. and −72.5° C. The reaction mixture became yellow slurry and was stirred for 37 min at −74° C. N,N-Dimethylformamide (60.6 mL, 783 mmol) was charged in one portion via a syringe and this addition produced an exotherm from −74.5° C. to −66.5° C. The reaction was monitored by HPLC at 90 min after the addition. The starting material was present at 2.9%. The cold bath was removed and the reaction mixture was warmed in ambient temperature. The reaction mixture was sampled and analyzed after 3 h and unreacted starting material was present at 1.5%. The reaction was deemed complete and was quenched by addition of the reaction solution to ice water (1.4 L) and diluted with ethyl acetate (1.5 L). The aqueous layer was extracted with ethyl acetate (1.5 L) and the organic layers were combined and washed with brine (20% w/w aq. NaCl, 2×600 mL) and dried over anhydrous MgSO₄. The MgSO₄ was removed by filtration and the filtrate was concentrated to an oil with some solids present. This residue was dissolved in methylene chloride and loaded onto a pad of silica gel (586 g). The silica pad was eluted with 2% EtOAc/DCM (monitored by TLC using 100% DCM as eluent). The desired fractions were collected and concentrated under reduced pressure to give a light amber oil. The oil was placed under high vacuum to give compound vi as a yellow solid (146.5 g, 95.1% yield). ¹H NMR (400 MHz, CDCl₃) δ 10.27 (s, 1H), 7.78 (d, J=8.5 Hz, 1H), 4.10-3.96 (m, 4H), 3.87-3.76 (m, 2H), 1.72 (s, 3H), 1.44 (t, J=7.0 Hz, 3H). LCMS for C₁₃H₁₅ClFO₄ (M+H)⁺: m/z=289.1.

### Example 3. Additional Alternative Synthesis of 3-chloro-6-ethoxy-2-fluoro-5-(2-methyl-1,3-dioxolan-2-yl)benzaldehyde

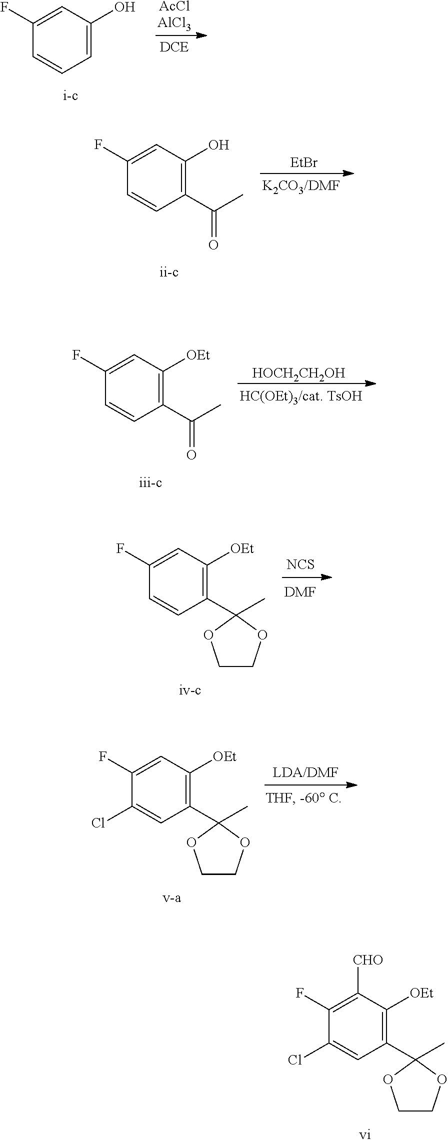

### Step 1. 1-(4-Fluoro-2-hydroxyphenyl)ethanone (ii-c)

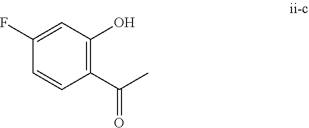

A reactor was charged with 3-fluoro-phenol (i-c, 225 kg) and dichloroethane (450 kg). The solution was heated to 40-45° C. and acetyl chloride (236.3 kg) was added, while the temperature was maintained at 40-50° C. After the addition, the reaction mixture was agitated at 50-70° C. for an additional 2 hrs. The reaction mixture was concentrated and the residue was dissolved in dichloroethane (450 kg). The solution was added to a mixture of aluminium chloride (320.1 kg) and dichloroethane (675 kg), while the temperature was maintained at 10-25° C. The resulting mixture was slowly added to dichloroethane (675 kg), while the temperature was kept at 75-85° C. After the addition, the resulting reaction mixture was stirred at 75-85° C. for an additional 4 hrs. The mixture was cooled to 10-30° C. and quenched into 1.1NHCl solution (1485 kg). The organic phase was separated and the aqueous phase was extracted with dichloroethane (250 kg). The organic extracts were combined, washed with half-saturated brine (1500 kg), dried over Na₂SO₄ (50 kg) and filtered. The filtrate was concentrated to afford 1-(4-fluoro-2-hydroxyphenyl)ethanone (ii-c, 230 Kg) as a yellow oil.

### Step 2. 1-(2-Ethoxy-4-fluorophenyl)ethanone (iii-c)

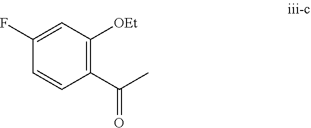

A reactor was charged with 1-(4-fluoro-2-hydroxyphenyl)ethanone (ii-c, 524.8 kg), DMF (1309 kg), K₂CO₃ (678.3 kg), and bromoethane (480.8 kg). The reaction mixture was agitated and heated at 50° C. for 8 hrs, at which time ion pair chromatography (IPC) showed NMT 1.0% of 1-(4-fluoro-2-hydroxyphenyl)ethanone (ii-c). The mixture was allowed to cool to room temperature and diluted with water (3927 kg). The precipitate was collected by filtration and washed with water (800 kg). The cake was dissolved in cyclohexane (2000 kg) heated at 60° C. and the bottom aqueous layer was drained off. The solution was cooled to 15° C., aged for 1 h and filtered to give 1-(2-ethoxy-4-fluorophenyl)ethanone (iii-c, 520 Kg) as a yellow solid.

### Step 3. 2-(2-Ethoxy-4-fluorophenyl)-2-methyl-1,3-dioxolane (iv-c)

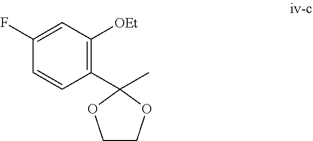 [SMILES: CCOc1cc(F)ccc1C1(C)OCCO1]

A reactor was charged with 1-(2-ethoxy-4-fluorophenyl)ethanone (iii-c, 520 kg), ethylene glycol (531 kg), cyclohexane (2600 kg) and p-toluenesulfonic acid monohydrate (27 kg). The mixture was heated under reflux for 72 h, while water was azotropically removed. The mixture was cooled and triethyl orthoformate (846 kg) was added. Heating under reflux was continued for an additional 4 hrs or until IPC confirmed NMT 5% of the starting material. The mixture was allowed to cool to 10-30° C. and then was added to a 2.8% Na₂CO₃ solution (2141 kg). The organic phase was separated and the aqueous phase was extracted with t-butyl methyl ether (MTBE) twice (1000 kg and 600 kg). The combined organic stream was washed with water twice (2×2080 kg), dried over Na₂SO₄ (50 kg) and filtered. The filtrate was concentrated to give crude 2-(2-ethoxy-4-fluorophenyl)-2-methyl-1,3-dioxolane (iv-c, 645.8 Kg) as a yellow oil, which was used directly for the next step without further purification.

### Step 4. 2-(5-Chloro-2-ethoxy-4-fluorophenyl)-2-methyl-1,3-dioxolane (v-a)

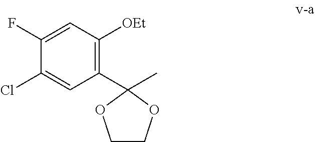 [SMILES: CCOc1cc(F)c(Cl)cc1C1(C)OCCO1]

A reactor was charged with 2-(2-ethoxy-4-fluorophenyl)-2-methyl-1,3-dioxolane (iv-c, 322.9 kg) and DMF (968.7 kg). N-chlorosuccinamide (NCS, 179 kg) was charged over the course of 1 h while the internal temperature was maintained at 15-20° C. The mixture was agitated for 12 hrs or until IPC indicated NMT 2.0% of the starting material (2-(2-ethoxy-4-fluorophenyl)-2-methyl-1,3-dioxolane, iv-c). The reaction mixture was added to a solution of Na₂SO₃ (36 kg) in water (2620 kg). The product solids were collected by filtration and rinsed washed with water (800 kg) and dried to afford 2-(5-chloro-2-ethoxy-4-fluorophenyl)-2-methyl-1,3-dioxolane (v-a, 329 Kg) as white solids. ¹H NMR (300 MHz, CDCl₃) δ7.51 (d, J=8.8 Hz, 1H), 6.70 (d, J=11.0 Hz, 1H), 4.17-3.92 (m, 4H), 3.91-3.80 (m, 2H), 1.75 (s, 3H), 1.46 (t, J=7.0 Hz, 3H).

### Step 5. 3-Chloro-6-ethoxy-2-fluoro-5-(2-methyl-1,3-dioxolan-2-yl)benzaldehyde (vi)

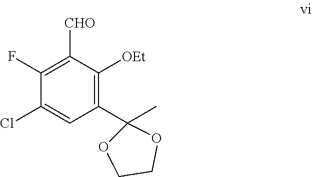 [SMILES: CCOc1c(C2(C)OCCO2)cc(Cl)c(F)c1C=O]

3-Chloro-6-ethoxy-2-fluoro-5-(2-methyl-1,3-dioxolan-2-yl)benzaldehyde (vi) was made from 2-(5-chloro-2-ethoxy-4-fluorophenyl)-2-methyl-1,3-dioxolane (v-a) by the method described in Example 2, step 3.

### Example 4. Synthesis of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) from 3-Chloro-6-ethoxy-2-fluoro-5-(2-methyl-1,3-dioxolan-2-yl)benzaldehyde (vi)

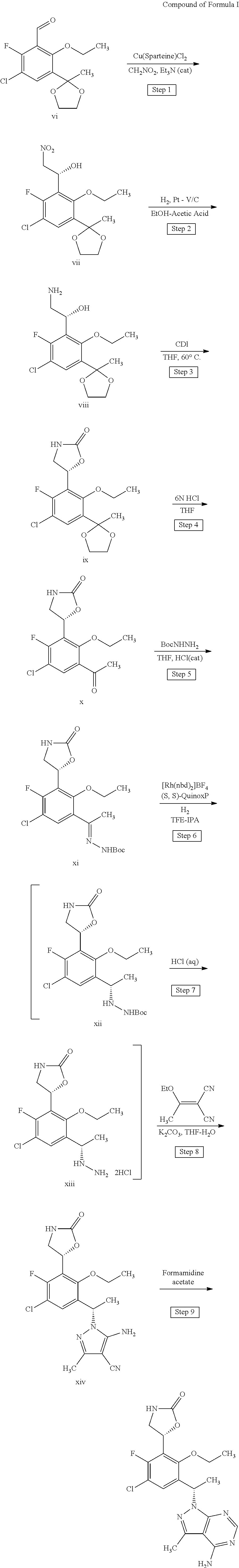

### Step 1. (R)-1-(3-chloro-6-ethoxy-2-fluoro-5-(2-methyl-1,3-dioxolan-2-yl)phenyl)-2-nitroethan-v-ol (vii)

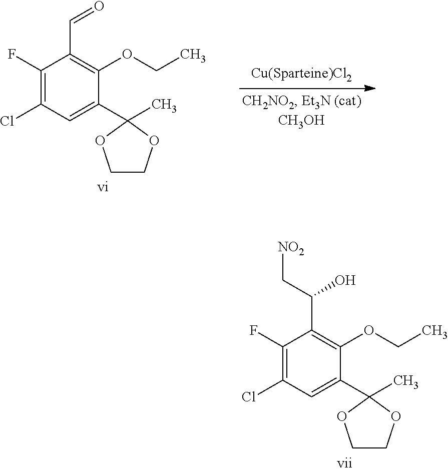 [SMILES: CCOc1c(C2(C)OCCO2)cc(Cl)c(F)c1C=O ; CCOc1c(C2(C)OCCO2)cc(Cl)c(F)c1[C@@H](O)C[N+](=O)[O-]]

Nitromethane (2626 ml, 48.5 mol) and methanol (875 mL) were degassed by purging with N₂. Cu[(−)-Sparteine]Cl₂ Complex (224 g, 0.606 mol) was added to the degassed mixture of nitromethane and methanol under N₂. The solution was degassed by sparging with N₂. 3-Chloro-6-ethoxy-2-fluoro-5-(2-methyl-1,3-dioxolan-2-yl)benzaldehyde (vi, 1750 g, 6.06 mol, see Examples 1, 2 or 3 for preparation) was added and the mixture was further sparged with N₂ for 20 min at rt. Triethylamine (8.45 ml, 60.6 mmol) was added at 0° C. The mixture was stirred under N₂ and gradually warmed up to rt. After 40-50 h, the reaction was complete, as judged by HPLC.

Most of the solvents were removed under reduced pressure. A mixture of methyl tert-butyl ether (MTBE) and n-heptane (1:1 v/v, 8.75 L) was added to precipitate the Cu catalyst, which was filtered off. The precipitated Cu[(−)-Sparteine]Cl₂ can be collected and reused. The filtrate was passed through a silica gel pad (packed with 1500 g silica gel) and the plug was washed with a mixture of MTBE and n-heptane (1:1 v/v). The filtrate was concentrated under reduced pressure to remove most of the MTBE. After ˜50% of MTBE was removed under reduced pressure, n-heptane (3.5 L) was added and it was concentrated until solid formed from the solution. The mixture was transferred to another 22 L flask and MTBE (0.85 L) was used to rinse and it was transferred to the flask. n-Heptane (15 L) was added to the flask and the mixture was agitated at rt overnight. The resulting solid was collected by filtration and washed with a mixture of 10% MTBE in n-heptane, then a mixture of 5% MTBE in n-heptane. The product was isolated as a light yellow solid (vii, 1660 g, 4.75 mol, 78% yield). ¹H NMR (400 MHz, CDCl₃) δ 7.65 (d, J=8.6 Hz, 1H), 5.86 (dd, J=10.1, 2.8 Hz, 1H), 5.19-5.08 (m, 1H), 4.56 (dd, J=13.6, 2.5 Hz, 1H), 4.26-3.97 (m, 4H), 3.97-3.77 (m, 2H), 3.08 (br s, 1H), 1.75 (s, 3H), 1.51 (t, J=7.0 Hz, 3H). ¹³C NMR (100 MHz, CDCl₃) δ 156.7, 154.4, 134.5, 129.4, 122.2, 116.7, 107.7, 79.0, 73.5, 64.6, 64.4, 25.8, 15.5. LCMS (ESI) m z calcd for C₁₄H₁₈ClFNO₆ (M+H⁺) 350.1, found 350.1. Enantiomeric purity: 97.5% ee (SFC)

### Step 2. (R)-2-amino-1-(3-chloro-6-ethoxy-2-fluoro-5-(2-methyl-1,3-dioxolan-2-yl)phenyl)ethan-1-ol (viii)

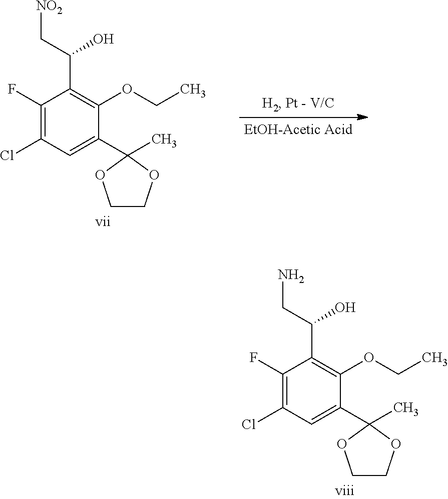 [SMILES: CCOc1c(C2(C)OCCO2)cc(Cl)c(F)c1[C@@H](O)C[N+](=O)[O-] ; CCOc1c(C2(C)OCCO2)cc(Cl)c(F)c1[C@@H](O)CN]

(R)-1-(3-Chloro-6-ethoxy-2-fluoro-5-(2-methyl-1,3-dioxolan-2-yl)phenyl)-2-nitroethan-1-ol (vii, 220 g, 623 mmol) was dissolved in a mixture of EtOH (1050 ml) and acetic acid (44 ml, 769 mmol). 1% Pt—2% V on activated carbon (55 g, 39 wt %) was charged to the solution and the mixture was agitated under 55 psi H₂ for 24-30 h until the reaction was complete.

The mixture was filtered through a pad of Celite and the cake was washed with EtOH (1050 mL). The filtrate was concentrated under reduced pressure to remove most of the organic solvents. THF (600 mL) was added to the residue and 1.0 N NaOH aqueous solution (780 mL, 780 mmol) was added to neutralize and basify the mixture under agitation. The resulting mixture was concentrated to remove most of the THF. After most of the organic solvents were evaporated, the solid was filtered and washed with water and MTBE to give the product as a white to light green solid (viii, 154 g, 482 mmol, 77% yield). ¹H NMR (400 MHz, CDCl₃) δ 7.49 (d, J=8.8 Hz, 1H), 4.90 (ddd, J=9.4, 3.9, 1.1 Hz, 1H), 4.06-4.00 (m, 2H), 3.98 (q, J=7.0 Hz, 2H), 3.87-3.80 (m, 1H), 3.80-3.73 (m, 1H), 3.20 (ddd, J=13.1, 9.5, 2.0 Hz, 1H), 2.93 (ddd, J=13.1, 3.9, 0.9 Hz, 1H), 2.49 (br s, 3H), 1.69 (s, 3H), 1.39 (t, J=7.0 Hz, 3H). ¹³C NMR (100 MHz, CDCl₃) δ 155.99, 154.5, 133.6, 127.8, 125.9, 116.4, 107.9, 72.9, 68.9, 64.5, 46.6, 25.8, 15.5. LCMS (ESI) m z calcd for C₁₄H₂₀ClFNO₄ (M+H⁺) 320.1, found 320.0.

### Step 3. (R)-5-(3-chloro-6-ethoxy-2-fluoro-5-(2-methyl-1,3-dioxolan-2-yl)phenyl)oxazolidin-2-one (ix)

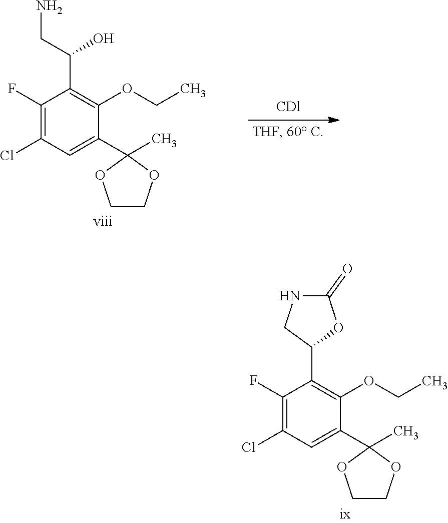 [SMILES: CCOc1c(C2(C)OCCO2)cc(Cl)c(F)c1[C@@H](O)CN ; CCOc1c(C2(C)OCCO2)cc(Cl)c(F)c1[C@@H]1CNC(=O)O1]

(R)-2-Amino-1-(3-chloro-6-ethoxy-2-fluoro-5-(2-methyl-1,3-dioxolan-2-yl)phenyl)ethan-1-ol (viiii, 900 g, 2.815 mol) was dissolved in THF (9 L) with agitation at 50° C. to give a clear solution. Carbonyldiimidazole (CDI) (228 g, 1.407 mol) was added to the solution and the solution was agitated at 56° C. for 10 min. Another portion of CDI (228 g, 1.407 mol) was added to the solution and it was agitated at 63° C. for 10 min. A final portion of CDI (2.28 g, 14.07 mmol) was added to the solution and it was agitated at 63° C. until reaction is complete.

The reaction mixture was transferred to a rotavapor flask and water (2 L) was added. The reaction mixture was concentrated to remove THF and a solid formed during concentration. Water (9 L) was added slowly during the concentration to maximize solid formation. After most of the THF was removed, the mixture was cooled to rt under agitation. The solid was collected by filtration and washed with water (9 L) and a mixture of MTBE and n-heptane (1:1 v/v, 2 L) to give the product as a white solid (ix). The product could be used for the next step without complete drying. ¹H NMR (400 MHz, CDCl₃) δ 7.60 (d, J=8.7 Hz, 1H), 6.28 (s, 1H), 5.92 (ddd, J=9.5, 8.1, 1.3 Hz, 1H), 4.09-3.71 (m, 8H), 1.70 (s, 3H), 1.42 (t, J=7.0 Hz, 3H). ¹³C NMR (100 MHz, CDCl₃) δ 159.5, 157.4, 155.0, 133.8, 129.6, 122.2, 116.8, 107.7, 73.7, 70.0, 64.6, 45.7, 25.8, 15.4. LCMS (ESI) m z calcd for C₁₅H₁₈ClFNO₅ (M+H⁺) 346.1, found 345.9. Enantiomeric purity: >99.9% ee (SFC)

### Step 4. (R)-5-(3-acetyl-5-chloro-2-ethoxy-6-fluorophenyl)oxazolidin-2-one (x)

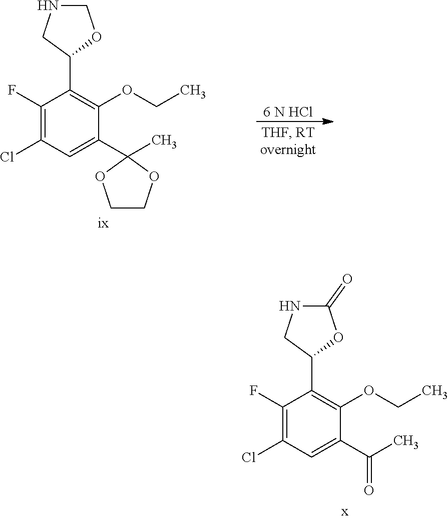

(R)-5-(3-Chloro-6-ethoxy-2-fluoro-5-(2-methyl-1,3-dioxolan-2-yl)phenyl)oxazolidin-2-one (ix, 2040 g wet weight, 5.900 mol theoretical) was suspended in THF (9 L). 12 N hydrochloric acid (2.5 L, 30.00 mol) was diluted with water (2.5 L) and added to the above suspension at rt. The reaction mixture was stirred overnight.

Sodium hydroxide (1.204 kg, 30.00 mol) was added to the reaction mixture slowly to neutralize excess hydrochloric acid. The reaction mixture was then diluted with water (4.5 L). The pH was determined to be −7 via pH test paper. The mixture was concentrated to remove THF and water (4.50 L) was added during the concentration process to precipitate a solid. The resulting solid was then filtered and washed with water (4.50 L) to give (R)-5-(3-acetyl-5-chloro-2-ethoxy-6-fluorophenyl)oxazolidin-2-one (x) as a white solid. The product was used for the next step without complete drying. ¹H NMR (500 MHz, DMSO) δ 7.89-7.85 (m, 2H), 5.95 (dd, J=9.8, 7.5 Hz, 1H), 3.94-3.89 (m, 1H), 3.88 (q, J=6.9 Hz, 2H), 3.55 (dd, J=8.9, 7.4 Hz, 1H), 2.58 (s, 3H), 1.32 (t, J=7.0 Hz, 3H). ¹³C NMR (125 MHz, DMSO) δ 198.0, 158.69, 158.68, 156.2, 131.7, 131.4, 123.3, 116.4, 74.1, 68.6, 45.5, 30.5, 15.4. LCMS (ESI) m/z calcd for C₁₃H₁₃ClFNO₄Na (M+Na⁺) 324.0, found 324.0. Enantiomeric purity: >99.9% ee (SFC)

### Step 5. tert-Butyl (R,E)-2-(1-(5-chloro-2-ethoxy-4-fluoro-3-(2-oxooxazolidin-5-yl)phenyl)ethylidene)hydrazine-1-carboxylate (xi)

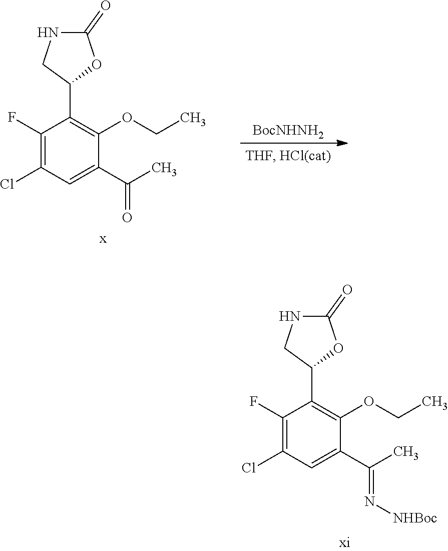 [SMILES: "BocNHNH2" ; CCOc1c(C(C)=O)cc(Cl)c(F)c1[C@@H]1CNC(=O)O1 ; CCOc1c(/C(C)=N/NC(=O)OC(C)(C)C)cc(Cl)c(F)c1[C@@H]1CNC(=O)O1]

(R)-5-(3-Acetyl-5-chloro-2-ethoxy-6-fluorophenyl)oxazolidin-2-one (x, 890 g, 2.950 mol) was dissolved in THF (10 L). tert-Butyl carbazate (520 g, 3.935 mol) and 1.0 N hydrochloric acid (0.088 L, 88 mmol) was added to the solution. The mixture was stirred at rt for 48 h. MTBE (4 L) and n-heptane (6 L) was added to the reaction mixture under agitation in 3 portions over 62 h. After the completion of the reaction, the ratio of E-hydrazone isomer to Z-hydrazone isomer was >95/5. The solid was collected via filtration and washed with a mixture of MTBE (4 L) and n-heptane (6 L). The solid was dried overnight to give a light yellow solid (xi, 1200 g, 2.886 mol, 98% yield). ¹H NMR (500 MHz, DMSO) δ 7.54 (d, J=8.5 Hz, 1H), 5.93 (dd, J=9.8, 7.5 Hz, 1H), 3.94-3.86 (m, 1H), 3.81 (q, J=6.9 Hz, 2H), 3.57-3.50 (m, 1H), 2.17 (s, 3H), 1.48 (s, 9H), 1.27 (t, J=6.9 Hz, 3H). ¹³C NMR (125 MHz, DMSO) δ 158.8, 156.6, 155.1, 153.6, 147.4, 132.3, 131.5, 122.7, 115.7, 80.1, 72.3, 68.8, 45.6, 28.6, 17.4, 15.5. LCMS (ESI) m z calcd for C₁₈H₂₃ClFN₃O₅Na (M+Na⁺) 438.1, found 438.1. Enantiomeric purity: >99.9% ee (SFC) E-hydrazone/Z-hydrazone: 97.5/2.5

### Step 6. tert-Butyl 2-((S)-1-(5-chloro-2-ethoxy-4-fluoro-3-((R)-2-oxooxazolidin-5-yl)phenyl)ethyl)hydrazine-1-carboxylate (xii)

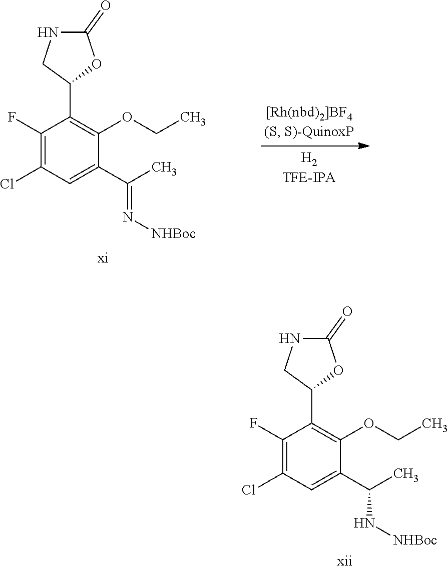 [SMILES: CCOc1c(/C(C)=N/NC(=O)OC(C)(C)C)cc(Cl)c(F)c1[C@@H]1CNC(=O)O1 ; CCOc1c([C@H](C)NNC(=O)OC(C)(C)C)cc(Cl)c(F)c1[C@@H]1CNC(=O)O1]

### Experimental Procedure at Low Hydrogen Pressure

Rh(nbd)₂BF₄ (16.29 g, 43.6 mmol) and 2,3-bis((S)-tert-butyl(methyl)phosphanyl)quinoxaline (16.02 g, 47.9 mmol) were added to a mixture of degassed trifluoroethanol (900 ml) and 2-propanol (100 ml). The reaction mixture was agitated and degassed with N₂. Tert-butyl (R,E)-2-(1-(5-chloro-2-ethoxy-4-fluoro-3-(2-oxooxazolidin-5-yl)phenyl)ethylidene)hydrazine-1-carboxylate (xi, 226.4 g, 544 mmol) was added to the reaction mixture and further degassed. The reaction mixture was agitated under 55-60 psi hydrogen atmosphere at ambient temperature for 40-44 h. The product solution (xii) was used for the next step without further purification or concentration of the solvents.

### Experimental Procedure at High Hydrogen Pressure

Rh(nbd)₂BF₄ (0.045 g, 0.120 mmol) and 2,3-bis((S)-tert-butyl(methyl)phosphanyl)quinoxaline (0.044 g, 0.132 mmol) was added to a mixture of degassed trifluoroethanol (54 ml) and 2-propanol (6 ml). The reaction mixture was agitated and degassed with N₂. Tert-butyl (R,E)-2-(1-(5-chloro-2-ethoxy-4-fluoro-3-(2-oxooxazolidin-5-yl)phenyl)ethylidene)hydrazine-1-carboxylate (xi, 20.0 g, 48.1 mmol) was added to the catalyst solution and further degassed under agitation. The reaction mixture was agitated under a 20 bar hydrogen atmosphere at 68-70° C. for 24-28 h. The product solution (xii) was used for the next step without further purification or concentration of the solvents.

LCMS (ESI) m z calcd for C₁₈H₂₅ClFN₃O₅Na (M+Na⁺) 440.1, found 440.1. Diastereomeric Purity: 92.0/8.0 dr (SFC, the diastereomer observed is tert-butyl 2-((R)-1-(5-chloro-2-ethoxy-4-fluoro-3-((R)-2-oxooxazolidin-5-yl)phenyl)ethyl)hydrazine-1-carboxylate))

### Step 7. (R)-5-(3-chloro-6-ethoxy-2-fluoro-5-((S)-1-hydrazinylethyl)phenyl)oxazolidin-2-one (xiii)

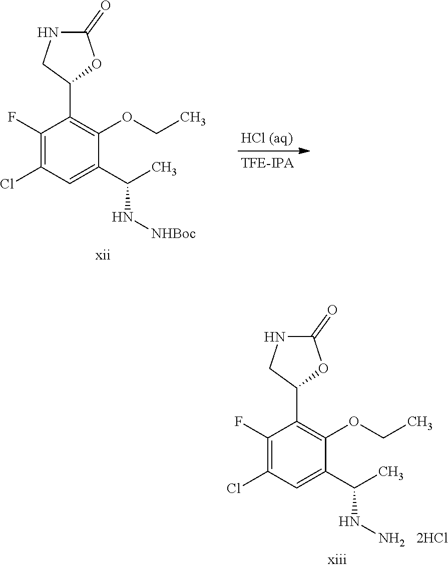 [SMILES: CCOc1c([C@H](C)NNC(=O)OC(C)(C)C)cc(Cl)c(F)c1[C@@H]1CNC(=O)O1 ; CCOc1c([C@H](C)NN)cc(Cl)c(F)c1[C@@H]1CNC(=O)O1]

tert-Butyl 2-((S)-1-(5-chloro-2-ethoxy-4-fluoro-3-((R)-2-oxooxazolidin-5-yl)phenyl)ethyl)hydrazine-1-carboxylate (xii, 1800 g theoratical yield from step 6, 4.308 mol) solution in a mixture of trifluoroethanol (6.84 L) and 2-propanol (0.76 L) was charged to a 22 L reactor. 6 N hydrochloric acid (3.159 L, 18.954 mol) was added to give a clear solution. The clear solution was agitated at 48-51° C. for 1 h. The solution was cooled to rt and MTBE (5 L) was added. The mixture was agitated for 10 min and the layers were separated. The organic layer was extracted with water for 4 times (1 L each time). The resulting organic layer was concentrated to remove some solvents. The final volume was reduced to ˜4.5 L. The resulting organic layer was extracted with 1 N hydrochloric acid 2 times (1 L each time). The combined aqueous layer was washed with MTBE 4 times (4 L each time). The aqueous solution of the product (xiii) as the HCl salt was used for next step without further purification or concentration. LCMS (ESI) m z calcd for C₁₃H₁₈ClFN₃O₃(M+H⁺) 318.1, found 318.1.

### Step 8. 5-Amino-1-((S)-1-(5-chloro-2-ethoxy-4-fluoro-3-((R)-2-oxooxazolidin-5-yl)phenyl)ethyl)-3-methyl-1H-pyrazole-4-carbonitrile (xiv)

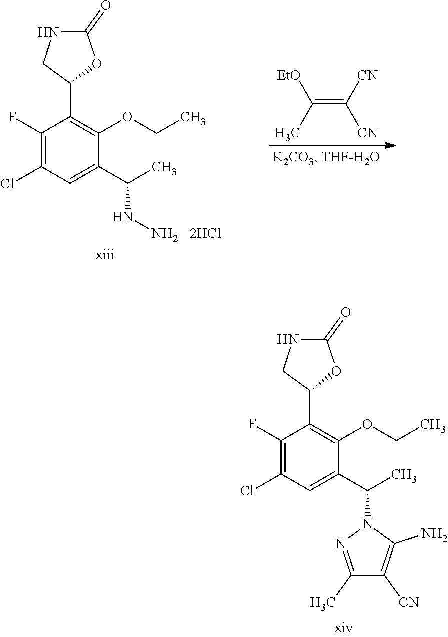

(R)-5-(3-Chloro-6-ethoxy-2-fluoro-5-((S)-1-hydrazinylethyl)phenyl)oxazolidin-2-one (xiii, 1369 g theoretical yield, 4.308 mol) in aqueous solution as a HCl salt was combined with THF (5 L). Potassium carbonate (2739 g, 19.80 mol) was added to neutralize and basify the solution to pH˜11, as determined by pH paper. 2-(1-Ethoxyethylidene)malononitrile (587 g, 4.308 mol) was then charged in to the reaction mixture. The biphasic mixture was agitated until the reaction was complete. CH₂Cl₂ (5 L) was added to the reaction mixture and it was further agitated for 10 min. The organic layer was separated. The organic layer was washed with 1 N NaOH aqueous solution (5 L), 0.5 N NaOH aqueous solution (4 L), and water (4 L), respectively. The resulting organic solution was concentrated under reduced pressure to remove most of the organic solvents. The crude product was recrystallized from isopropyl acetate (4 L) and n-heptane (4 L) to give INCB101297 as a yellow solid (xiv, 1300 g, 3.188 mol, 74% yield for 3 steps based on xi). ¹H NMR (400 MHz, DMSO) δ 7.84 (s, 1H), 7.64 (d, J=8.4 Hz, 1H), 6.62 (s, 2H), 5.87-5.81 (m, 1H), 5.68 (q, J=6.8 Hz, 1H), 3.89 (t, J=9.4 Hz, 1H), 3.73 (dq, J=8.7, 6.9 Hz, 1H), 3.62-3.52 (m, 2H), 2.05 (s, 3H), 1.59 (d, J=6.8 Hz, 3H), 1.38 (t, J=6.9 Hz, 3H). ¹³C NMR (100 MHz, DMSO) δ 158.6, 156.75, 154.7, 151.6, 149.2, 132.0, 130.8, 122.4, 116.2, 115.7, 73.3, 69.1, 48.4, 45.4, 20.4, 15.6, 13.3. LCMS (ESI) m z calcd for C₁₈H₂₀ClFN₅O₃(M+H⁺) 408.1, found 408.1. Diastereomeric Purity: 99.2/0.8 dr (SFC, the diastereomer observed is 5-amino-1-((R)-1-(5-chloro-2-ethoxy-4-fluoro-3-((R)-2-oxooxazolidin-5-yl)phenyl)ethyl)-3-methyl-H-pyrazole-4-carbonitrile)). Enantiomeric purity: >99.9% ee (SFC)

### Step 9. (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Compound of Formula I)

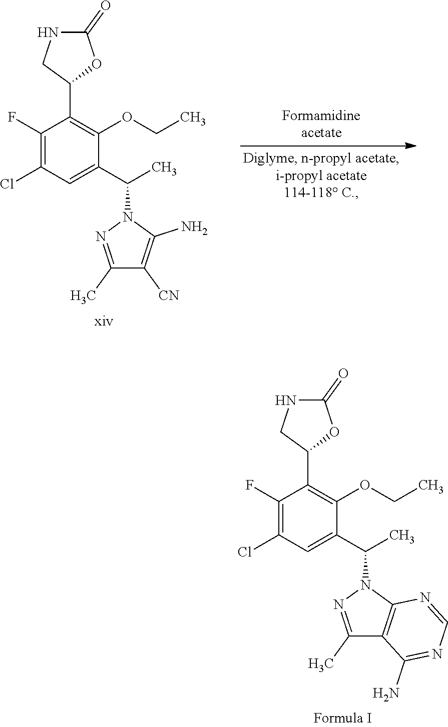 [SMILES: CCOc1c([C@H](C)n2nc(C)c(C#N)c2N)cc(Cl)c(F)c1[C@@H]1CNC(=O)O1 ; CCOc1c([C@H](C)n2nc(C)c3c(N)ncnc32)cc(Cl)c(F)c1[C@@H]1CNC(=O)O1]

(R)-5-Amino-1-((S)-1-(5-chloro-2-ethoxy-4-fluoro-3-((R)-2-oxooxazolidin-5-yl)phenyl)ethyl)-3-methyl-1H-pyrazole-4-carbonitrile (xiv, 1119 g, 2.744 mol) was added to a mixture of isopropyl acetate (0.966 L), n-propyl acetate (1.25 L) and diglyme (1.55 L). Formimidamide acetate (1714 g, 16.50 mol) was added to the mixture and it was heated to 110-120° C. under reflux for 7.5 h. The mixture was cooled by adding water (3 L). Ethyl acetate (5.4 L) was then added to the reaction mixture. The reaction mixture was filtered through a celite pad to remove insolubles. The layers were separated and the organic layer was washed with water 3 times (3 L each time). The combined aqueous layers were extracted with ethyl acetate (5.4 L) 2 times. The resulting organic layer was washed with water 2 times (3 L each time). The organic layers were combined and added to 1 N HCl (3 L). The mixture was agitated, the aqueous layer was separated and the organic layer was extracted with 1 N HCl 3 times (1 L each time). The combined aqueous layers were washed with ethyl acetate (5.4 L). The resulting aqueous layers were added to CH₂Cl₂ (4 L). The biphasic mixture was neutralized by NaOH (274 g, 6.859 mol) dissolved in ice water. The pH of the aqueous layer was 5-6 by pH test paper. The organic layers were separated and washed with 1 N NaOH 3 times (2 L each time). The resulting organic layer was treated with activated charcoal (500 g). The organic solution was filtered through a celite pad and washed with CH₂Cl₂(14 L). The filtrate was concentrated to remove all the CH₂Cl₂. The crude product was recrystallized from a mixture of ethyl acetate, MTBE and n-heptane to give (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) as a crystalline solid. ¹H NMR (600 MHz, DMSO) δ 8.13 (s, 1H), 7.84 (s, 1H), 7.70 (d, J=8.3 Hz, 1H), 6.25 (q, J=7.0 Hz, 1H), 5.89-5.84 (m, 1H), 3.95-3.85 (m, 2H), 3.81 (dq, J=9.1, 6.9 Hz, 1H), 3.52 (t, J=8.3 Hz, 1H), 2.55 (s, 3H), 1.73 (d, J=7.1 Hz, 3H), 1.40 (t, J=6.9 Hz, 3H). ¹³C NMR (150 MHz, DMSO) δ 158.9, 158.6, 156.5, 156.3, 154.3, 154.1, 141.6, 133.6, 130.1, 122.6, 116.4, 99.4, 73.50, 69.1, 48.3, 45.5, 21.5, 15.7, 15.0. LCMS (ESI) m/z calcd for C₁₉H₂₁ClFN₆O₃(M+H⁺) 435.1, found 435.1. Diastereomeric Purity: >99.9% dr (chiral HPLC). Enantiomeric purity: >99.9% ee (chiral HPLC). The water content in the product was determined to be 0.26% by Karl Fischer titration.

### Example 5. Alternative Synthesis of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) from 3-Chloro-6-ethoxy-2-fluoro-5-(2-methyl-1,3-dioxolan-2-yl)benzaldehyde (vi)

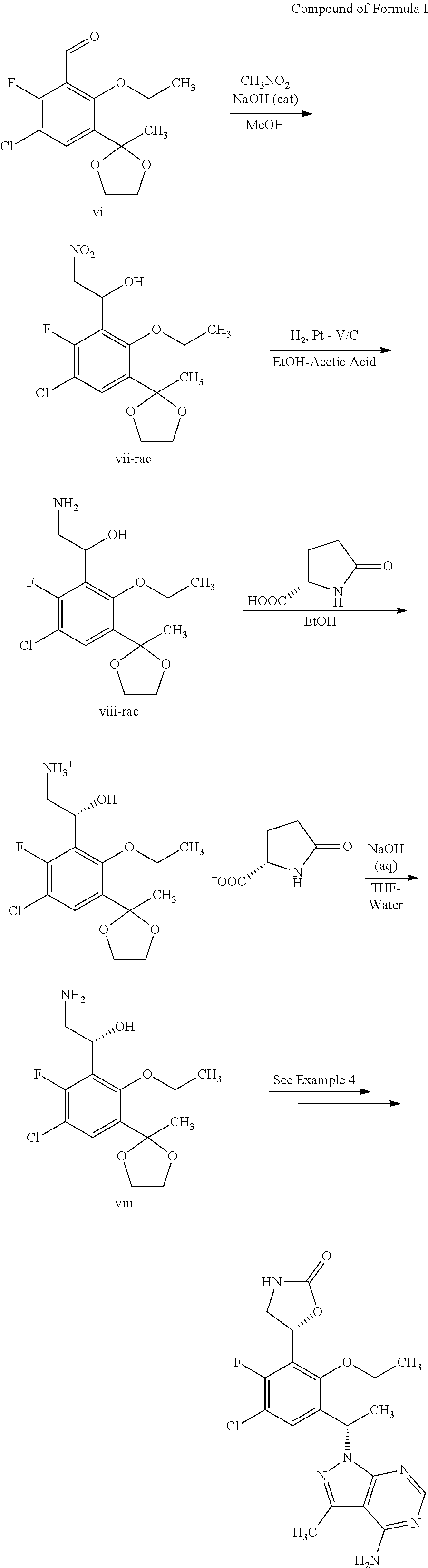

### Step 1. 1-(3-chloro-6-ethoxy-2-fluoro-5-(2-methyl-1,3-dioxolan-2-yl)phenyl)-2-nitroethan-1-ol (vii-rac)

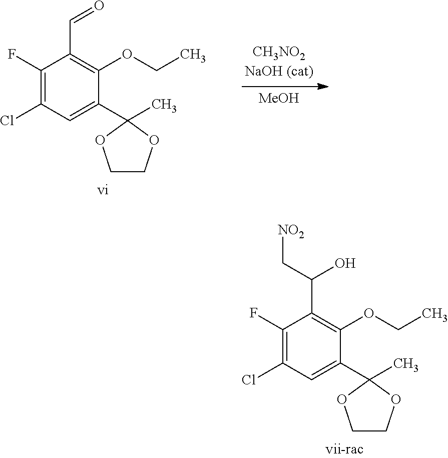 [SMILES: "MeOH" ; CCOc1c(C2(C)OCCO2)cc(Cl)c(F)c1C=O ; CCOc1c(C2(C)OCCO2)cc(Cl)c(F)c1C(O)C[N+](=O)[O-]]

3-Chloro-6-ethoxy-2-fluoro-5-(2-methyl-1,3-dioxolan-2-yl)benzaldehyde (vi, 290 g, 1.00 mol) was added to a mixture of nitromethane (218 mL, 4.02 mol) and methanol (400 mL). Sodium hydroxide solution (1.0 N, 100 mL, 0.10 mol) was charged dropwise to the reaction mixture. The solution was agitated at ambient temperature for 1 h.

The solution was concentrated under reduced pressure to remove most of the organic solvents. Hexanes (600 mL) was charged to the residue under agitation. A solid precipitated from the biphasic solution and was filtered and washed with water and hexanes to give crude 1-(3-chloro-6-ethoxy-2-fluoro-5-(2-methyl-1,3-dioxolan-2-yl)phenyl)-2-nitroethan-1-ol (vii-rac) as a yellow solid, which was used for the next step without further purification.

### Step 2. 2-Amino-1-(3-chloro-6-ethoxy-2-fluoro-5-(2-methyl-1,3-dioxolan-2-yl)phenyl)ethan-1-ol (viii-rac)

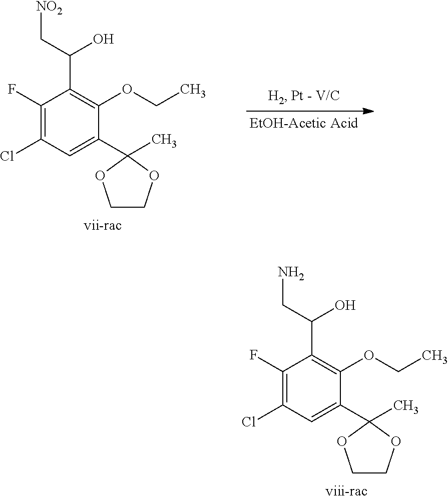 [SMILES: CCOc1c(C2(C)OCCO2)cc(Cl)c(F)c1C(O)C[N+](=O)[O-] ; CCOc1c(C2(C)OCCO2)cc(Cl)c(F)c1C(O)CN]

The 2-Amino-1-(3-chloro-6-ethoxy-2-fluoro-5-(2-methyl-1,3-dioxolan-2-yl)phenyl)ethan-1-ol (viii-rac) was prepared from 1-(3-chloro-6-ethoxy-2-fluoro-5-(2-methyl-1,3-dioxolan-2-yl)phenyl)-2-nitroethan-1-ol (vii-rac) using the same procedure described in Example 4, step 2 for the preparation of (R)-2-amino-1-(3-chloro-6-ethoxy-2-fluoro-5-(2-methyl-1,3-dioxolan-2-yl)phenyl)ethan-1-ol (viii).

### Step 3. (R)-2-amino-1-(3-chloro-6-ethoxy-2-fluoro-5-(2-methyl-1,3-dioxolan-2-yl)phenyl)ethan-1-ol (viii)

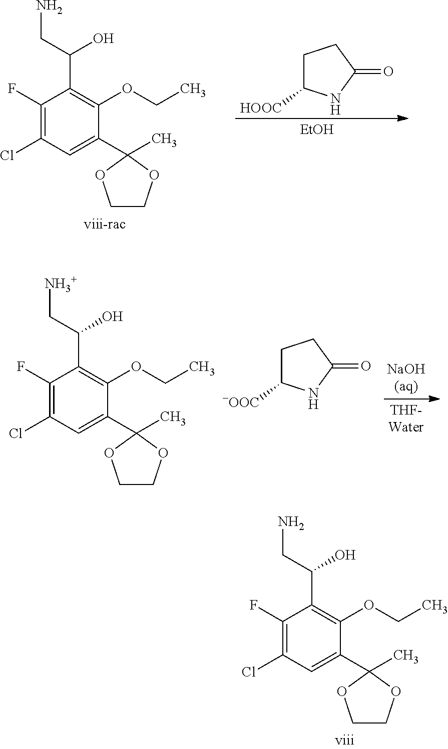

2-Amino-1-(3-chloro-6-ethoxy-2-fluoro-5-(2-methyl-1,3-dioxolan-2-yl)phenyl)ethan-1-ol (viii-rac, 142 g, 435 mmol) was dissolved in ethanol (2092 ml) under mild heating. (S)-5-oxopyrrolidine-2-carboxylic acid (28.1 g, 218 mmol) was added in one portion as a solid. The mixture was agitated at ambient temperature. The salt slowly precipitated from the solution. After agitating the mixture for 24 h, the precipitate was filtered and washed with EtOH (150 mL for 2 times). The solid was dried briefly in a vacuum oven.

The solid was then dissolved in 350 mL THF and 350 mL water. Aqueous NaOH (1.0 N, 220 mL, 220 mmol) was added to neutralize and basify the solution. The solution became cloudy after the addition of the base, once the pH reached 14. The mixture was concentrated under reduced pressure to remove most of the THF. A white solid precipitated during concentration. After most of the THF was removed, the white solid was filtered and washed with water. The solid was dried in a vacuum oven to give viii as a white solid (48.3 g, 34.7% yield based on racemic free base).

Alternatively, the (R)-2-(3-chloro-6-ethoxy-2-fluoro-5-(2-methyl-1,3-dioxolan-2-yl)phenyl)-2-hydroxyethan-1-aminium (S)-5-oxopyrrolidine-2-carboxylate could also be formed in a mixture of THF and ethanol as solvents. 2-Amino-1-(3-chloro-6-ethoxy-2-fluoro-5-(2-methyl-1,3-dioxolan-2-yl)phenyl)ethan-1-ol (viii-rac, 65.0 g, 203 mmol) was dissolved in ethanol (215 ml) and THE (430 mL). (S)-5-oxopyrrolidine-2-carboxylic acid (13.4 g, 104 mmol) was added in one portion as a solid. The mixture was agitated at ambient temperature for 24 h. The solid was filtered and washed a mixture of THE and MTBE (1:1 v/v, 130 mL) and MTBE (130 mL). The salt obtained was neutralized following the same procedure described above to yield viii as a white solid (25.0 g, 41% yield based on racemic free base).

### Synthesis of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Compound of Formula I)

(R)-5-(3-(S)-1-(4-amino-3-methyl-1H-pyrazolo[3,4-d]pyrimidin-1-yl)ethyl)-5-chloro-2-ethoxy-6-fluorophenyl)oxazolidin-2-one was synthesized from (R)-2-amino-1-(3-chloro-6-ethoxy-2-fluoro-5-(2-methyl-1,3-dioxolan-2-yl)phenyl)ethan-1-ol (viii) following the procedures described in Steps 3-9 of Example 4.

### Example 6. Synthesis and Characterization of Hydrochloride Salt of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I)

125.22 mg of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) free base was dissolved in 2 mL of 1:1 dichloromethane (DCM)/methanol in a 4 mL clear glass vial with stirring. 317 μL of 1M aqueous HCl (1.1 eq) was added to the solution and mixed well. The solution was evaporated without a cap at room temperature to dryness. To the resulting solid, 1 mL of methylethyl ketone (MEK) was added and stirred for 2 hours at room temperature. The solid of hydrochloride salt was collected by filtration and air dried. The salt ratio between the free base and hydrochloric acid was determined to be 1.0 by chloride titration.

The hydrochloride salt was confirmed as a crystalline solid by XRPD analysis. The XRPD pattern of the hydrochloride salt is shown in FIG. 1 and the peak data is given below in Table 2.

TABLE 2
XRPD Peak Data for Hydrochloride Salt.

| 2-Theta (°) | Relative Intensity (%) |
|---|---|
| 7.4 | 2.7 |
| 9.3 | 0.9 |
| 10.2 | 53.2 |
| 10.7 | 100 |
| 12.3 | 1.9 |
| 13.5 | 4.9 |
| 14.3 | 3.6 |
| 14.7 | 52.3 |
| 16.1 | 3.4 |
| 17.1 | 1.6 |
| 17.3 | 2.6 |
| 17.6 | 14.3 |
| 18.2 | 35.4 |
| 18.8 | 15.3 |
| 19.6 | 34.3 |
| 19.9 | 28.9 |
| 20.5 | 21.5 |
| 21.5 | 30.8 |
| 22.0 | 38.3 |
| 22.3 | 31.0 |
| 23.8 | 10.5 |
| 24.0 | 10.0 |
| 25.0 | 5.4 |
| 25.8 | 7.4 |
| 26.4 | 33.6 |
| 27.3 | 4.9 |
| 28.1 | 12.4 |
| 29.6 | 5.4 |

DSC analysis of the hydrochloride salt revealed a first endothermic peak with an onset temperature of 49.5° C. and a maximum at 68.1° C., a second endothermic peak with an onset temperature of 134.1° C. and a maximum at 150.9° C. and a third endothermic peak with an onset temperature of 219.8° C. and a maximum at 232.9° C. An exothermic peak was also observed around 175-225° C. The DSC thermogram is provided in FIG. 2.

TGA analysis of the hydrochloride salt revealed 1.5% weight loss below 75° C., 4.9% weight loss between 80° C. and 180° C. and significant weight loss above 180° C. due to decomposition of the sample. The TGA thermogram is provided in FIG. 3.

### Example 7. Synthesis and Characterization of Phosphate Salt of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I)

150.77 mg of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) free base was dissolved in 3 mL of 1:1 dichloromethane (DCM)/methanol in a 4 mL clear glass vial with stirring. 381 μL of 1M aqueous phosphoric acid (1.1 eq) was added to the solution and mixed well. The solution was evaporated without a cap at room temperature to dryness. To the resulting solid, 1 mL of methylethyl ketone (MEK) was added and stirred for 2 hours at room temperature. The solid phosphate salt was collected by filtration and air dried. The salt ratio between the free base and phosphoric acid was determined to be 1.25 by NMR analysis.

The phosphate salt was confirmed as a crystalline solid by XRPD analysis. The XRPD pattern of the phosphate salt is shown in FIG. 4 and the peak data is given below in Table 3.

TABLE 3
XRPD Peak Data for Phosphate Salt.

| 2-Theta (°) | Relative Intensity (%) |
|---|---|
| 5.7 | 1.5 |
| 6.8 | 16.3 |
| 7.2 | 1.4 |
| 7.7 | 10.9 |
| 8.7 | 2.9 |
| 9.1 | 11.0 |
| 10.8 | 52.3 |
| 11.1 | 91.3 |
| 11.3 | 100 |
| 12.1 | 26.0 |
| 13.4 | 32.3 |
| 14.3 | 12.5 |
| 14.6 | 39.1 |
| 15.4 | 34.1 |
| 15.6 | 59.2 |
| 17.7 | 50.1 |
| 18.1 | 47.0 |
| 18.3 | 46.1 |
| 18.6 | 66.3 |
| 18.9 | 28.8 |
| 19.4 | 24.5 |
| 19.8 | 31.6 |
| 20.1 | 27.3 |
| 20.6 | 18.7 |
| 20.8 | 24.7 |
| 21.1 | 64.4 |
| 21.7 | 19.0 |
| 22.3 | 54.8 |
| 22.9 | 48.7 |
| 23.5 | 47.7 |
| 23.7 | 58.3 |
| 24.4 | 34.6 |
| 24.7 | 30.9 |
| 25.1 | 55.8 |
| 25.4 | 23.1 |
| 25.8 | 13.6 |
| 26.6 | 29.3 |
| 26.8 | 29.6 |
| 27.4 | 18.7 |
| 27.6 | 25.5 |
| 28.2 | 15.7 |
| 28.6 | 14.1 |
| 28.9 | 18.1 |

DSC analysis of the phosphate salt revealed a first endothermic peak with an onset temperature of 37.9° C. and a maximum at 90.8° C., a second endothermic peak with an onset temperature of 126.1° C. and a maximum at 131.0° C. and a third endothermic peak with a maximum temperature of 239.1° C. The DSC thermogram is provided in FIG. 5.

TGA analysis of the phosphate salt revealed 5.8% weight loss below 100° C. and 15.9% weight loss between 100° C. and 300° C. The TGA thermogram is provided in FIG. 6.

### Example 8. Synthesis and Characterization of Maleate Salt of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I)

108.15 mg of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) free base was dissolved in 2 mL of 1:1 dichloromethane (DCM)/methanol in a 4 mL clear glass vial with stirring. 32.03 mg of maleic acid (1.1 eq) was added to the solution and mixed well. The solution was evaporated without a cap at room temperature to dryness. To the resulting solid, 1 mL of methanol was added and stirred for 2 hours at room temperature. The solid of the maleate salt was collected by filtration and air dried. The salt ratio between the free base and maleic acid was determined to be 1.0 by NMR analysis.

The maleate salt was confirmed as a crystalline solid by XRPD analysis. The XRPD pattern of the maleate salt is shown in FIG. 7 and the peak data is given below in Table 4.

TABLE 4
XRPD Peak Data for Maleate Salt.

| 2-Theta (°) | Relative Intensity (%) |
|---|---|
| 7.2 | 16.3 |
| 9.0 | 18.9 |
| 10.1 | 11.4 |
| 10.3 | 12.0 |
| 10.8 | 17.3 |
| 11.4 | 23.6 |
| 11.6 | 21.3 |
| 12.8 | 13.2 |
| 12.9 | 11.1 |
| 13.1 | 7.2 |
| 14.0 | 8.4 |
| 14.4 | 34.7 |
| 14.9 | 100 |
| 16.2 | 59.8 |
| 17.6 | 30.6 |
| 17.8 | 38.8 |
| 17.9 | 36.9 |
| 19.2 | 13.8 |
| 19.4 | 13.0 |
| 20.7 | 38.8 |
| 21.0 | 28.9 |
| 21.5 | 36.0 |
| 21.8 | 34.0 |
| 24.0 | 32.1 |
| 25.2 | 92.1 |
| 25.7 | 82.1 |
| 27.3 | 22.6 |
| 28.0 | 45.8 |
| 28.8 | 8.4 |
| 28.9 | 11.1 |

DSC analysis of the maleate salt revealed a first endothermic peak with an onset temperature of 47.5° C. and a maximum at 72.1° C., a second endothermic peak with an onset temperature of 148.4° C. and a maximum at 157.7° C. and a third endothermic peak with a maximum temperature of 184.0° C. The DSC thermogram is provided in FIG. 8.

TGA analysis of the maleate salt revealed 2.6% weight loss below 75° C., 9.5% weight loss between 125° C. and 200° C., and significant weight loss above 200° C. due to decomposition of the sample. The TGA thermogram is provided in FIG. 9.

### Example 9. Synthesis and Characterization of Tosylate Salt of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I)

110.3 mg of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) free base was dissolved in 2 mL of 1:1 dichloromethane (DCM)/methanol in a 4 mL clear glass vial with stirring. 54.90 mg of p-toluenesulfonic acid (1.1 eq) was added to the solution and mixed well. The solution was evaporated without a cap at room temperature to dryness. To the resulting oil, 1 mL of ethyl acetate was added and stirred for 2 hours at room temperature. The solid of the tosylate salt was collected by filtration and air dried. The salt ratio between the free base and toluenesulfonic acid was determined to be 1.0 by NMR analysis.

The tosylate salt was confirmed as a crystalline solid by XRPD analysis. The XRPD pattern of tosylate salt is shown in FIG. 10 and the peak data is given below in Table 5.

TABLE 5
XRPD Peak Data for Tosylate Salt.

| 2-Theta (°) | Relative Intensity (%) |
|---|---|
| 8.8 | 26.0 |
| 10.6 | 0.9 |
| 10.8 | 3.0 |
| 11.1 | 2.5 |
| 11.9 | 37.2 |
| 12.2 | 13.2 |
| 13.1 | 2.4 |
| 13.5 | 8.7 |
| 15.4 | 2.6 |
| 16.2 | 18.7 |
| 17.0 | 100 |
| 17.7 | 89.1 |
| 19.7 | 10.8 |
| 20.2 | 14.2 |
| 20.4 | 11.4 |
| 21.1 | 4.2 |
| 22.0 | 19.9 |
| 22.4 | 31.8 |
| 23.6 | 46.4 |
| 24.3 | 27.0 |
| 27.5 | 21.2 |
| 28.1 | 14.9 |
| 28.2 | 16.2 |
| 29.4 | 1.5 |
| 29.5 | 1.7 |

DSC analysis of the tosylate salt revealed a first endothermic peak with an onset temperature of 102.3° C. and a maximum at 130.4° C. and a second endothermic peak with an onset temperature of 213.1° C. and a maximum at 215.8° C. The DSC thermogram is provided in FIG. 11.

TGA analysis of the tosylate salt revealed 2.5% weight loss below 150° C. and 9.5% weight loss between 175° C. and 300° C. The TGA thermogram is provided in FIG. 12.

### Example 10. Solid State Characterization of Crystalline Free Base (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) Form I

### X-Ray Powder Diffraction (XRPD)

The crystalline free base of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) Form I, as synthesized in Example 4, was characterized by XRPD. The X-Ray Powder Diffraction (XRPD) was obtained from a Bruker D2 PHASER X-ray Powder Diffractometer (XRPD) instrument. The general experimental procedures for XRPD were: (1) X-ray radiation from copper at 1.054056 Å with K_{β} filter and LYNXEYE™ detector; (2) X-ray power at 30 kV, 10 mA; and (3) the sample powder was dispersed on a zero-background sample holder. The general measurement conditions for XRPD were: Start Angle 5 degrees; Stop Angle 30 degrees; Sampling 0.015 degrees; and Scan speed 2 degree/min.

The XRPD pattern of crystalline free base Form I is shown in FIG. 13 and the peak data is given below in Table 6.

TABLE 6
XRPD Peak Data for Crystalline Free Base Form I

| 2-Theta (°) | Relative Intensity (%) |
|---|---|
| 8.3 | 0.3 |
| 9.2 | 51.6 |
| 10.4 | 1.1 |
| 11.5 | 100 |
| 12.8 | 0.4 |
| 13.2 | 4.5 |
| 13.7 | 8.1 |
| 14.2 | 42.3 |
| 15.1 | 26.2 |
| 15.7 | 9.1 |
| 16.7 | 16.3 |
| 17.3 | 12.8 |
| 17.8 | 9.4 |
| 18.4 | 1.1 |
| 19.3 | 0.7 |
| 20.0 | 17.8 |
| 20.3 | 84.5 |
| 20.7 | 35.6 |
| 21.4 | 60.7 |
| 21.9 | 3.4 |
| 22.0 | 5.3 |
| 22.6 | 7.6 |
| 23.0 | 80.5 |
| 23.6 | 15.7 |
| 24.5 | 14.5 |
| 25.2 | 5.1 |
| 25.5 | 0.5 |
| 25.9 | 4.2 |
| 26.4 | 3.9 |
| 26.6 | 7.1 |
| 27.6 | 36.8 |
| 28.5 | 15.2 |
| 28.9 | 6.6 |
| 29.8 | 0.6 |

The crystalline free base of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) Form I was characterized by DSC. The DSC was obtained from TA Instruments Differential Scanning Calorimetry, Model Q2000 with autosampler. The DSC instrument conditions were as follows: 25-300° C. at 10° C./min; Tzero aluminum sample pan and lid; and nitrogen gas flow at 50 mL/min. DSC analysis of crystalline free base Form I revealed one small endothermic peak with an onset temperature of 190.7° C. and a maximum at 192.2° C. and a second major endothermic peak with an onset temperature of 252.7° C. and a maximum at 254.8° C. The DSC thermogram is provided in FIG. 14.

The crystalline free base of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) Form I was characterized by TGA. The TGA was obtained from PerkinElmer Thermogravimetric Analyzer, Model Pyris 1. The general experimental conditions for TGA were: ramp from 25° C. to 350° C. at 10° C./min; nitrogen purge gas flow at 60 mL/min; ceramic crucible sample holder. TGA analysis of crystalline free base Form I revealed significant weight loss of 12.5% between 200° C. and 300° C. due to decomposition of the sample. The TGA thermogram is provided in FIG. 15.

### Example 11. Characterization of Single Crystal (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I) hydrochloric acid salt

### Crystal Data

C22 H27 Cl2 F N6 O4, from acetone, colorless, irregular block, ˜0.450×0.340×0.340 mm, triclinic, P1, a=8.775 (6) Å, b=11.799 (7) Å, c=13.732 (9) Å, alpha=111.667 (10)°, beta=98.849 (11)°, gamma=90.346 (10) °, Vol=1302.6 (14) Å³, Z=2, T=−40.° C., Formula weight=529.39, Density=1.350 g/cm³, μ(Mo)=0.30 mm⁻¹.

### Data Collection

Data collection was performed using a Bruker SMART APEX-II CCD system, MoKalpha radiation, standard focus tube, anode power=50 kV×30 mA, crystal to plate distance=5.0 cm, 512×512 pixels/frame, beam center=(259.19, 253.13), total frames=2254, oscillation/frame=0.50°, exposure/frame=30.1 sec/frame, SAINT integration, hkl min/max=(−10, 11, −15, 15, −18, 18), data input to shelx=23753, unique data=23753, two-theta range=3.91 to 56.83°, completeness to two-theta 56.83=99.90%, R(int-xl)=0.0000, SADABS correction applied.

### Solution and Refinement

The crystal structure was solved using XS(Shelxtl) and refined using shelxtl software package. Refinement was by full-matrix least squares on F², scattering factors from Int. Tab. Vol C Tables 4.2.6.8 and 6.1.1.4, number of data=23753, number of restraints=3, number of parameters=642, data/parameter ratio=37.00, goodness-of-fit on F²=1.01, R indices[I>4sigma(I)] R1=0.0389, wR2=0.0926, R indices (all data) R1=0.0575, wR2=0.1031, max difference peak and hole=0.267 and −0.274 e/Å³, refined flack parameter=0.01(2). All of the hydrogen atoms were idealized using a riding model. Table 7 shows atomic coordinates (×10⁴) and equivalent isotropic displacement parameters (Å²×103). U(eq) is defined as one third of the trace of the orthogonalized Uij tensor. Table 8 shows bond lengths [Å] and angles [deg]. Table 9 shows anisotropic displacement parameters (Å²×10³).

### Results

This analysis determines the structure of a chloride salt of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one (Formula I). The asymmetric unit contained two molecules of the compound of Formula I, two chlorides to balance the charge and two acetone solvent molecules as shown in FIG. 16 with thermal ellipsoids drawn to the 30% probability level. The predicted structure was confirmed. The data crystal was a non-merohedral twin and both domains of data were used for the final refinement. The enantiomeric setting was based on the Flack parameter that refined to 0.015(23). This analysis confirmed the absolute configuration at the chiral centers of C1=S, C11=R, C21=S and C31=R.

TABLE 7
Atomic Coordinates

| | x | y | z | U(eq) |
|---|---|---|---|---|
| Cl(1)A1:E61 | 5374(2) | 159(1) | 1624(1) | 72(1) |
| Cl(21) | 2016(2) | 9962(1) | −1626(2) | 75(1) |
| Cl(22) | −513(2) | 8943(1) | 5173(1) | 54(1) |
| Cl(23) | 4436(2) | 1045(1) | 4847(1) | 51(1) |
| F(1) | 2377(4) | −261(2) | 2153(2) | 55(1) |
| F(21) | −1244(4) | 10269(2) | −2183(2) | 53(1) |
| O(1) | −66(4) | 2999(3) | 1460(3) | 48(1) |
| O(2) | −873(5) | −184(3) | 1667(3) | 48(1) |
| O(3) | −1606(5) | −1750(3) | 2079(3) | 61(1) |
| O(21) | −3244(4) | 6940(3) | −1486(3) | 44(1) |
| O(22) | −4196(5) | 10126(3) | −1649(3) | 50(1) |
| O(23) | −5058(5) | 11735(4) | −2010(4) | 68(1) |
| O(40) | 3602(9) | 6232(5) | 4816(4) | 111(2) |
| O(43) | 8632(8) | 3694(6) | 5166(4) | 112(2) |
| N(1) | −637(7) | 83(4) | 3362(4) | 61(1) |
| N(2) | 3770(5) | 4766(3) | 1714(3) | 40(1) |
| N(3) | 2060(6) | 6342(4) | 2407(3) | 43(1) |
| N(4) | 3432(5) | 8220(3) | 3463(3) | 42(1) |
| N(5) | 5333(5) | 4632(3) | 1808(3) | 45(1) |
| N(6) | 6076(6) | 8608(4) | 3862(4) | 56(1) |
| N(21) | −4805(7) | 9905(4) | −3329(4) | 62(1) |
| N(22) | 449(5) | 5251(3) | −1759(3) | 41(1) |
| N(23) | −1608(6) | 3682(4) | −2345(4) | 50(1) |
| N(24) | −800(6) | 1781(3) | −3429(3) | 47(1) |
| N(25) | 1948(5) | 5384(3) | −1896(3) | 44(1) |
| N(26) | 1612(6) | 1398(4) | −3884(4) | 53(1) |
| C(1) | 2660(7) | 3803(4) | 945(4) | 40(1) |
| C(2) | 3033(10) | 3498(5) | −179(5) | 57(2) |
| C(3) | 2591(6) | 2689(4) | 1246(3) | 36(1) |
| C(4) | 3844(7) | 2012(4) | 1288(4) | 43(1) |
| C(5) | 3769(7) | 1007(4) | 1573(4) | 44(1) |
| C(6) | 2443(6) | 711(4) | 1857(4) | 41(1) |
| C(7) | 1144(6) | 1358(4) | 1840(3) | 37(1) |
| C(8) | 1225(6) | 2335(4) | 1500(3) | 36(1) |
| C(9) | −1189(12) | 2473(7) | 513(9) | 142(5) |
| C(10) | −1800(16) | 3168(10) | 29(8) | 132(5) |
| C(11) | −252(7) | 1086(5) | 2251(4) | 48(1) |
| C(12) | −1078(7) | −708(5) | 2380(5) | 46(1) |
| C(13) | 70(8) | 1208(4) | 3428(4) | 56(2) |
| C(14) | 3494(6) | 5916(4) | 2302(4) | 36(1) |
| C(15) | 2120(7) | 7492(4) | 3002(4) | 47(1) |
| C(16) | 4879(7) | 7824(4) | 3389(4) | 38(1) |
| C(17) | 4908(6) | 6558(4) | 2783(4) | 36(1) |
| C(18) | 6037(6) | 5707(4) | 2460(4) | 42(1) |
| C(19) | 7750(7) | 5872(5) | 2758(6) | 61(2) |
| C(21) | −285(8) | 6224(4) | −980(4) | 47(1) |
| C(22) | 687(11) | 6563(6) | 129(5) | 70(2) |
| C(23) | −524(7) | 7313(4) | −1296(4) | 37(1) |
| C(24) | 729(7) | 8047(5) | −1325(4) | 47(1) |
| C(25) | 477(7) | 9032(4) | −1615(4) | 44(1) |
| C(26) | −1007(6) | 9292(4) | −1889(4) | 39(1) |
| C(27) | −2292(6) | 8611(4) | −1871(3) | 37(1) |
| C(28) | −2023(6) | 7629(4) | −1543(3) | 36(1) |
| C(29) | −3770(11) | 7442(6) | −456(6) | 88(2) |
| C(30) | −4390(12) | 6525(8) | −148(6) | 90(3) |
| C(31) | −3890(7) | 8875(5) | −2248(4) | 46(1) |
| C(32) | −4734(7) | 10685(5) | −2329(5) | 49(1) |
| C(33) | −4170(8) | 8751(5) | −3427(4) | 55(2) |
| C(34) | −155(7) | 4105(4) | −2302(4) | 39(1) |
| C(35) | −1848(8) | 2519(5) | −2925(4) | 53(1) |
| C(36) | 661(7) | 2184(4) | −3400(4) | 40(1) |
| C(37) | 1016(6) | 3450(4) | −2820(4) | 37(1) |
| C(38) | 2308(6) | 4309(4) | −2542(4) | 39(1) |
| C(39) | 3856(7) | 4123(5) | −2865(5) | 57(2) |
| C(40) | 2412(10) | 5850(6) | 4974(6) | 71(2) |
| C(41) | 1705(14) | 4634(9) | 4216(7) | 136(4) |
| C(42) | 1660(13) | 6521(9) | 5884(8) | 117(3) |
| C(43) | 7433(10) | 4131(6) | 4999(5) | 72(2) |
| C(44) | 6239(13) | 3484(8) | 4077(7) | 115(3) |
| C(45) | 7101(13) | 5350(8) | 5767(6) | 118(3) |

TABLE 8
Bond lengths and bond angles

| | Bond Length (Å) |
|---|---|
| Cl(1)—C(5) | 1.740(6) |
| Cl(21)—C(25) | 1.741(6) |
| F(1)—C(6) | 1.355(5) |
| F(21)—C(26) | 1.362(5) |
| O(1)—C(8) | 1.387(6) |
| O(1)—C(9) | 1.432(8) |
| O(2)—C(12) | 1.369(6) |
| O(2)—C(11) | 1.467(6) |
| O(3)—C(12) | 1.207(6) |
| O(21)—C(28) | 1.371(6) |
| O(21)—C(29) | 1.466(7) |
| O(22)—C(32) | 1.360(6) |
| O(22)—C(31) | 1.451(6) |
| O(23)—C(32) | 1.204(6) |
| O(40)—C(40) | 1.216(9) |
| O(43)—C(43) | 1.202(9) |
| N(1)—C(12) | 1.321(7) |
| N(1)—C(13) | 1.428(7) |
| N(2)—C(14) | 1.344(6) |
| N(2)—N(5) | 1.373(6) |
| N(2)—C(1) | 1.462(7) |
| N(3)—C(15) | 1.296(6) |
| N(3)—C(14) | 1.367(7) |
| N(4)—C(15) | 1.352(7) |
| N(4)—C(16) | 1.361(7) |
| N(5)—C(18) | 1.331(6) |
| N(6)—C(16) | 1.310(7) |
| N(21)—C(32) | 1.334(7) |
| N(21)—C(33) | 1.443(7) |
| N(22)—C(34) | 1.339(6) |
| N(22)—N(25) | 1.374(6) |
| N(22)—C(21) | 1.480(6) |
| N(23)—C(35) | 1.304(6) |
| N(23)—C(34) | 1.351(8) |
| N(24)—C(36) | 1.354(7) |
| N(24)—C(35) | 1.358(7) |
| N(25)—C(38) | 1.326(6) |
| N(26)—C(36) | 1.313(6) |
| C(1)—C(3) | 1.521(6) |
| C(1)—C(2) | 1.538(8) |
| C(3)—C(4) | 1.370(7) |
| C(3)—C(8) | 1.401(7) |
| C(4)—C(5) | 1.382(7) |
| C(5)—C(6) | 1.365(7) |
| C(6)—C(7) | 1.378(7) |
| C(7)—C(8) | 1.399(6) |
| C(7)—C(11) | 1.505(8) |
| C(9)—C(10) | 1.303(10) |
| C(11)—C(13) | 1.550(7) |
| C(14)—C(17) | 1.385(7) |
| C(16)—C(17) | 1.420(6) |
| C(17)—C(18) | 1.416(7) |
| C(18)—C(19) | 1.488(8) |
| C(21)—C(23) | 1.507(6) |
| C(21)—C(22) | 1.534(9) |
| C(23)—C(28) | 1.396(7) |
| C(23)—C(24) | 1.410(8) |
| C(24)—C(25) | 1.370(7) |
| C(25)—C(26) | 1.368(8) |
| C(26)—C(27) | 1.390(7) |
| C(27)—C(28) | 1.398(6) |
| C(27)—C(31) | 1.495(8) |
| C(29)—C(30) | 1.431(9) |
| C(31)—C(33) | 1.550(8) |
| C(34)—C(37) | 1.410(7) |
| C(36)—C(37) | 1.415(7) |
| C(37)—C(38) | 1.423(7) |
| C(38)—C(39) | 1.486(8) |
| C(40)—C(42) | 1.467(11) |
| C(40)—C(41) | 1.488(10) |
| C(43)—C(44) | 1.467(11) |
| C(43)—C(45) | 1.498(10) |
| | Bond Angle (°) |
| C(8)—O(1)—C(9) | 114.8(5) |
| C(12)—O(2)—C(11) | 109.0(4) |
| C(28)—O(21)—C(29) | 112.6(4) |
| C(32)—O(22)—C(31) | 109.8(4) |
| C(12)—N(1)—C(13) | 114.0(4) |
| C(14)—N(2)—N(5) | 110.2(4) |
| C(14)—N(2)—C(1) | 127.7(4) |
| N(5)—N(2)—C(1) | 121.5(4) |
| C(15)—N(3)—C(14) | 112.5(5) |
| C(15)—N(4)—C(16) | 124.0(4) |
| C(18)—N(5)—N(2) | 107.3(4) |
| C(32)—N(21)—C(33) | 114.0(5) |
| C(34)—N(22)—N(25) | 111.5(4) |
| C(34)—N(22)—C(21) | 125.4(5) |
| N(25)—N(22)—C(21) | 122.6(4) |
| C(35)—N(23)—C(34) | 111.8(5) |
| C(36)—N(24)—C(35) | 123.2(4) |
| C(38)—N(25)—N(22) | 107.4(4) |
| N(2)—C(1)—C(3) | 111.6(4) |
| N(2)—C(1)—C(2) | 109.6(5) |
| C(3)—C(1)—C(2) | 112.9(4) |
| C(4)—C(3)—C(8) | 118.1(4) |
| C(4)—C(3)—C(1) | 121.7(5) |
| C(8)—C(3)—C(1) | 120.1(4) |
| C(3)—C(4)—C(5) | 121.1(5) |
| C(6)—C(5)—C(4) | 119.5(5) |
| C(6)—C(5)—Cl(1) | 119.9(4) |
| C(4)—C(5)—Cl(1) | 120.6(5) |
| F(1)—C(6)—C(5) | 119.6(4) |
| F(1)—C(6)—C(7) | 118.1(5) |
| C(5)—C(6)—C(7) | 122.3(4) |
| C(6)—C(7)—C(8) | 117.1(5) |
| C(6)—C(7)—C(11) | 121.2(4) |
| C(8)—C(7)—C(11) | 121.5(4) |
| O(1)—C(8)—C(7) | 118.8(4) |
| O(1)—C(8)—C(3) | 119.5(4) |
| C(7)—C(8)—C(3) | 121.7(4) |
| C(10)—C(9)—O(1) | 119.5(7) |
| O(2)—C(11)—C(7) | 111.2(4) |
| O(2)—C(11)—C(13) | 104.2(4) |
| C(7)—C(11)—C(13) | 114.4(5) |
| O(3)—C(12)—N(1) | 129.0(5) |
| O(3)—C(12)—O(2) | 120.7(5) |
| N(1)—C(12)—O(2) | 110.3(4) |
| N(1)—C(13)—C(11) | 101.6(4) |
| N(2)—C(14)—N(3) | 125.0(5) |
| N(2)—C(14)—C(17) | 107.7(5) |
| N(3)—C(14)—C(17) | 127.3(4) |
| N(3)—C(15)—N(4) | 125.1(5) |
| N(6)—C(16)—N(4) | 119.2(4) |
| N(6)—C(16)—C(17) | 126.7(5) |
| N(4)—C(16)—C(17) | 114.1(5) |
| C(14)—C(17)—C(18) | 105.7(4) |
| C(14)—C(17)—C(16) | 116.8(5) |
| C(18)—C(17)—C(16) | 137.4(5) |
| N(5)—C(18)—C(17) | 109.1(5) |
| N(5)—C(18)—C(19) | 121.3(5) |
| C(17)—C(18)—C(19) | 129.6(5) |
| N(22)—C(21)—C(23) | 111.6(4) |
| N(22)—C(21)—C(22) | 109.1(5) |
| C(23)—C(21)—C(22) | 112.5(5) |
| C(28)—C(23)—C(24) | 118.6(4) |
| C(28)—C(23)—C(21) | 119.5(5) |
| C(24)—C(23)—C(21) | 121.8(5) |
| C(25)—C(24)—C(23) | 120.6(5) |
| C(26)—C(25)—C(24) | 119.4(5) |
| C(26)—C(25)—Cl(21) | 119.7(4) |
| C(24)—C(25)—Cl(21) | 120.9(5) |
| F(21)—C(26)—C(25) | 118.9(4) |
| F(21)—C(26)—C(27) | 118.2(5) |
| C(25)—C(26)—C(27) | 122.9(4) |
| C(26)—C(27)—C(28) | 117.3(5) |
| C(26)—C(27)—C(31) | 121.1(4) |
| C(28)—C(27)—C(31) | 121.5(5) |
| O(21)—C(28)—C(23) | 118.8(4) |
| O(21)—C(28)—C(27) | 120.0(5) |
| C(23)—C(28)—C(27) | 121.2(5) |
| C(30)—C(29)—O(21) | 113.3(6) |
| O(22)—C(31)—C(27) | 110.7(4) |
| O(22)—C(31)—C(33) | 105.2(4) |
| C(27)—C(31)—C(33) | 113.5(5) |
| O(23)—C(32)—N(21) | 128.6(5) |
| O(23)—C(32)—O(22) | 121.6(5) |
| N(21)—C(32)—O(22) | 109.7(5) |
| N(21)—C(33)—C(31) | 100.6(4) |
| N(22)—C(34)—N(23) | 126.0(5) |
| N(22)—C(34)—C(37) | 106.3(5) |
| N(23)—C(34)—C(37) | 127.7(5) |
| N(23)—C(35)—N(24) | 126.1(6) |
| N(26)—C(36)—N(24) | 119.1(4) |
| N(26)—C(36)—C(37) | 126.0(5) |
| N(24)—C(36)—C(37) | 115.0(5) |
| C(34)—C(37)—C(36) | 116.2(5) |
| C(34)—C(37)—C(38) | 105.9(4) |
| C(36)—C(37)—C(38) | 137.9(5) |
| N(25)—C(38)—C(37) | 109.0(5) |
| N(25)—C(38)—C(39) | 121.8(5) |
| C(37)—C(38)—C(39) | 129.2(4) |
| O(40)—C(40)—C(42) | 123.1(8) |
| O(40)—C(40)—C(41) | 118.1(8) |
| C(42)—C(40)—C(41) | 118.8(9) |
| O(43)—C(43)—C(44) | 121.9(8) |
| O(43)—C(43)—C(45) | 119.9(8) |
| C(44)—C(43)—C(45) | 118.2(8) |

TABLE 9
Anisotropic displacement parameters

| | U11 | U22 | U33 | U23 | U13 | U12 |
|---|---|---|---|---|---|---|
| Cl(1) | 52(1) | 55(1) | 108(1) | 28(1) | 14(1) | 27(1) |
| Cl(21) | 50(1) | 56(1) | 114(1) | 24(1) | 25(1) | −10(1) |
| Cl(22) | 59(1) | 41(1) | 54(1) | 14(1) | 3(1) | −4(1) |
| Cl(23) | 61(1) | 39(1) | 52(1) | 13(1) | 13(1) | 9(1) |
| F(1) | 62(2) | 41(2) | 71(2) | 32(1) | 8(2) | 14(2) |
| F(21) | 61(2) | 38(1) | 70(2) | 28(1) | 20(2) | 5(1) |
| O(1) | 36(2) | 40(2) | 67(2) | 25(2) | −2(2) | 7(2) |
| O(2) | 47(2) | 48(2) | 52(2) | 23(2) | 2(2) | −6(2) |
| O(3) | 72(3) | 45(2) | 66(2) | 21(2) | 14(2) | −11(2) |
| O(21) | 45(2) | 38(2) | 52(2) | 18(2) | 16(2) | 0(2) |
| O(22) | 49(3) | 54(2) | 55(2) | 24(2) | 18(2) | 22(2) |
| O(23) | 65(3) | 55(2) | 91(3) | 31(2) | 23(2) | 31(2) |
| O(40) | 100(5) | 123(4) | 90(4) | 23(3) | 6(4) | −38(4) |
| O(43) | 98(5) | 129(5) | 94(4) | 23(3) | 17(4) | 51(4) |
| N(1) | 87(4) | 51(3) | 45(3) | 16(2) | 17(3) | −14(3) |
| N(2) | 35(3) | 32(2) | 52(2) | 15(2) | 5(2) | −2(2) |
| N(3) | 41(3) | 39(2) | 43(2) | 9(2) | 2(2) | 6(2) |
| N(4) | 43(3) | 30(2) | 45(2) | 7(2) | 3(2) | 7(2) |
| N(5) | 37(3) | 33(2) | 61(3) | 14(2) | 8(2) | 0(2) |
| N(6) | 40(3) | 31(2) | 82(3) | 4(2) | 9(2) | 1(2) |
| N(21) | 76(4) | 53(3) | 56(3) | 22(2) | 1(3) | 20(3) |
| N(22) | 49(3) | 33(2) | 42(2) | 13(2) | 15(2) | 14(2) |
| N(23) | 50(3) | 44(2) | 63(3) | 24(2) | 22(3) | 6(2) |
| N(24) | 51(3) | 35(2) | 57(3) | 16(2) | 14(2) | −2(2) |
| N(25) | 47(3) | 36(2) | 47(2) | 14(2) | 10(2) | 8(2) |
| N(26) | 48(3) | 34(2) | 67(3) | 4(2) | 16(2) | 2(2) |
| C(1) | 42(3) | 31(2) | 44(3) | 13(2) | 3(3) | −1(2) |
| C(2) | 82(5) | 46(3) | 41(3) | 15(3) | 7(3) | −10(3) |
| C(3) | 43(3) | 27(2) | 34(2) | 8(2) | 3(2) | 3(2) |
| C(4) | 43(3) | 33(2) | 52(3) | 12(2) | 15(3) | 5(2) |
| C(5) | 41(3) | 34(3) | 53(3) | 11(2) | 6(3) | 11(2) |
| C(6) | 43(3) | 28(2) | 48(3) | 13(2) | 1(2) | 6(2) |
| C(7) | 33(3) | 33(2) | 43(2) | 14(2) | 3(2) | 0(2) |
| C(8) | 39(3) | 26(2) | 34(2) | 6(2) | −3(2) | 1(2) |
| C(9) | 107(8) | 67(5) | 201(10) | 47(6) | −114(8) | −17(5) |
| C(10) | 159(11) | 123(7) | 100(7) | 59(6) | −63(7) | −38(8) |
| C(11) | 45(4) | 38(3) | 66(4) | 25(3) | 9(3) | 5(3) |
| C(12) | 42(3) | 43(3) | 59(3) | 23(3) | 16(3) | 6(3) |
| C(13) | 72(5) | 39(3) | 56(3) | 12(2) | 20(3) | 0(3) |
| C(14) | 42(3) | 32(2) | 37(3) | 15(2) | 6(2) | 2(2) |
| C(15) | 43(3) | 45(3) | 49(3) | 15(2) | 3(3) | 9(3) |
| C(16) | 38(3) | 31(2) | 41(3) | 12(2) | 4(2) | 2(2) |
| C(17) | 37(3) | 30(2) | 41(3) | 15(2) | 6(2) | 0(2) |
| C(18) | 35(3) | 30(2) | 61(3) | 16(2) | 11(3) | 0(2) |
| C(19) | 42(4) | 40(3) | 91(5) | 15(3) | 9(4) | 2(3) |
| C(21) | 59(4) | 43(3) | 43(3) | 17(2) | 19(3) | 23(3) |
| C(22) | 92(6) | 70(4) | 48(4) | 21(3) | 13(4) | 37(4) |
| C(23) | 40(3) | 28(2) | 37(3) | 5(2) | 6(2) | 6(2) |
| C(24) | 38(3) | 44(3) | 55(3) | 11(2) | 13(3) | 11(3) |
| C(25) | 37(3) | 35(3) | 57(3) | 10(2) | 14(3) | 2(2) |
| C(26) | 49(4) | 27(2) | 46(3) | 16(2) | 13(2) | 5(2) |
| C(27) | 37(3) | 32(2) | 39(2) | 10(2) | 6(2) | 1(2) |
| C(28) | 42(3) | 29(2) | 34(2) | 8(2) | 9(2) | 2(2) |
| C(29) | 113(7) | 77(4) | 95(5) | 35(4) | 72(5) | 10(4) |
| C(30) | 89(6) | 127(6) | 55(4) | 39(4) | 8(4) | −42(5) |
| C(31) | 41(3) | 42(3) | 57(3) | 22(2) | 5(3) | 0(3) |
| C(32) | 38(3) | 52(3) | 65(4) | 28(3) | 17(3) | 14(3) |
| C(33) | 54(4) | 43(3) | 60(3) | 17(3) | −11(3) | −1(3) |
| C(34) | 48(4) | 34(3) | 41(3) | 19(2) | 10(3) | 9(3) |
| C(35) | 52(4) | 45(3) | 69(4) | 24(3) | 21(3) | 4(3) |
| C(36) | 46(3) | 37(2) | 38(3) | 16(2) | 8(2) | 6(3) |
| C(37) | 39(3) | 34(2) | 39(3) | 13(2) | 7(2) | 7(2) |
| C(38) | 41(3) | 36(2) | 40(2) | 14(2) | 6(2) | 8(2) |
| C(39) | 43(4) | 46(3) | 69(4) | 6(3) | 15(3) | 4(3) |
| C(40) | 67(5) | 82(5) | 73(4) | 41(4) | 2(4) | −1(4) |
| C(41) | 125(9) | 136(8) | 106(7) | 8(6) | −8(7) | −67(8) |
| C(42) | 138(10) | 104(6) | 123(7) | 45(5) | 56(7) | 32(6) |
| C(43) | 73(6) | 80(4) | 63(4) | 23(4) | 21(4) | 15(4) |
| C(44) | 128(9) | 96(6) | 104(6) | 31(5) | −10(6) | −8(6) |
| C(45) | 113(8) | 120(7) | 88(6) | 2(5) | 11(6) | 22(7) |

TABLE 10
Hydrogen Coordinates and Isotropic Displacement Parameters

| | x | y | z | U(eq) |
|---|---|---|---|---|
| H(1) | −766 | −68 | 3919 | 73 |
| H(4) | 3347 | 8988 | 3830 | 50 |
| H(6A) | 5941 | 9370 | 4218 | 67 |
| H(6B) | 7007 | 8365 | 3821 | 67 |
| H(21) | −5199 | 10077 | −3875 | 74 |
| H(24) | −1080 | 1012 | −3788 | 57 |
| H(26A) | 1300 | 631 | −4217 | 64 |
| H(26B) | 2553 | 1645 | −3872 | 64 |
| H(1A) | 1626 | 4134 | 956 | 48 |
| H(2A) | 2267 | 2887 | −691 | 86 |
| H(2B) | 4050 | 3182 | −213 | 86 |
| H(2C) | 3018 | 4231 | −341 | 86 |
| H(4A) | 4771 | 2234 | 1120 | 52 |
| H(9A) | −708 | 1822 | 0 | 170 |
| H(9B) | −2037 | 2081 | 689 | 170 |
| H(10A) | −2753 | 2763 | −436 | 198 |
| H(10B) | −1083 | 3319 | −388 | 198 |
| H(10C) | −2019 | 3938 | 551 | 198 |
| H(11) | −1059 | 1640 | 2166 | 57 |
| H(13A) | 1182 | 1280 | 3697 | 67 |
| H(13B) | −410 | 1912 | 3882 | 67 |
| H(15) | 1177 | 7849 | 3121 | 56 |
| H(19A) | 8138 | 6483 | 2523 | 91 |
| H(19B) | 8195 | 5103 | 2422 | 91 |
| H(19C) | 8033 | 6140 | 3524 | 91 |
| H(21A) | −1313 | 5886 | −966 | 56 |
| H(22A) | 154 | 7134 | 654 | 105 |
| H(22B) | 1683 | 6939 | 148 | 105 |
| H(22C) | 839 | 5831 | 287 | 105 |
| H(24A) | 1747 | 7858 | −1143 | 56 |
| H(29A) | −2899 | 7903 | 89 | 106 |
| H(29B) | −4564 | 8013 | −490 | 106 |
| H(30A) | −4755 | 6913 | 523 | 134 |
| H(30B) | −3592 | 5986 | −67 | 134 |
| H(30C) | −5244 | 6055 | −689 | 134 |
| H(31) | −4645 | 8313 | −2153 | 55 |
| H(33A) | −3203 | 8662 | −3715 | 66 |
| H(33B) | −4906 | 8059 | −3871 | 66 |
| H(35) | −2839 | 2158 | −3001 | 64 |
| H(39A) | 3739 | 3621 | −3616 | 85 |
| H(39B) | 4471 | 3718 | −2457 | 85 |
| H(39C) | 4369 | 4909 | −2733 | 85 |
| H(41A) | 633 | 4709 | 3956 | 204 |
| H(41B) | 1756 | 4062 | 4575 | 204 |
| H(41C) | 2264 | 4340 | 3623 | 204 |
| H(42A) | 687 | 6790 | 5634 | 176 |
| H(42B) | 2328 | 7227 | 6364 | 176 |
| H(42C) | 1469 | 5992 | 6256 | 176 |
| H(44A) | 6642 | 2753 | 3614 | 172 |
| H(44B) | 5944 | 4012 | 3692 | 172 |
| H(44C) | 5343 | 3257 | 4317 | 172 |
| H(45A) | 6524 | 5795 | 5380 | 177 |
| H(45B) | 8067 | 5812 | 6156 | 177 |
| H(45C) | 6498 | 5232 | 6262 | 177 |

TABLE 11
Torsion Angles

| | Torsion Angle (°) |
|---|---|
| C(14)—N(2)—N(5)—C(18) | 0.0(5) |
| C(1)—N(2)—N(5)—C(18) | 171.8(4) |
| C(34)—N(22)—N(25)—C(38) | 0.3(5) |
| C(21)—N(22)—N(25)—C(38) | 172.1(4) |
| C(14)—N(2)—C(1)—C(3) | −120.7(5) |
| N(5)—N(2)—C(1)—C(3) | 69.0(6) |
| C(14)—N(2)—C(1)—C(2) | 113.3(6) |
| N(5)—N(2)—C(1)—C(2) | −56.9(6) |
| N(2)—C(1)—C(3)—C(4) | −63.3(6) |
| C(2)—C(1)—C(3)—C(4) | 60.8(7) |
| N(2)—C(1)—C(3)—C(8) | 116.1(5) |
| C(2)—C(1)—C(3)—C(8) | −119.8(6) |
| C(8)—C(3)—C(4)—C(5) | 0.0(7) |
| C(1)—C(3)—C(4)—C(5) | 179.4(4) |
| C(3)—C(4)—C(5)—C(6) | −2.5(8) |
| C(3)—C(4)—C(5)—Cl(1) | −179.9(4) |
| C(4)—C(5)—C(6)—F(1) | −178.9(4) |
| Cl(1)—C(5)—C(6)—F(1) | −1.5(7) |
| C(4)—C(5)—C(6)—C(7) | 2.0(8) |
| Cl(1)—C(5)—C(6)—C(7) | 179.4(4) |
| F(1)—C(6)—C(7)—C(8) | −178.1(4) |
| C(5)—C(6)—C(7)—C(8) | 1.0(7) |
| F(1)—C(6)—C(7)—C(11) | 6.6(7) |
| C(5)—C(6)—C(7)—C(11) | −174.3(5) |
| C(9)—O(1)—C(8)—C(7) | −83.3(7) |
| C(9)—O(1)—C(8)—C(3) | 99.4(7) |
| C(6)—C(7)—C(8)—O(1) | 179.2(4) |
| C(11)—C(7)—C(8)—O(1) | −5.5(6) |
| C(6)—C(7)—C(8)—C(3) | −3.6(6) |
| C(11)—C(7)—C(8)—C(3) | 171.7(4) |
| C(4)—C(3)—C(8)—O(1) | −179.7(4) |
| C(1)—C(3)—C(8)—O(1) | 0.9(6) |
| C(4)—C(3)—C(8)—C(7) | 3.2(6) |
| C(1)—C(3)—C(8)—C(7) | −176.3(4) |
| C(8)—O(1)—C(9)—C(10) | −135.0(12) |
| C(12)—O(2)—C(11)—C(7) | 130.1(4) |
| C(12)—O(2)—C(11)—C(13) | 6.4(6) |
| C(6)—C(7)—C(11)—O(2) | −59.5(6) |
| C(8)—C(7)—C(11)—O(2) | 125.4(5) |
| C(6)—C(7)—C(11)—C(13) | 58.2(6) |
| C(8)—C(7)—C(11)—C(13) | −116.9(5) |
| C(13)—N(1)—C(12)—O(3) | 174.5(6) |
| C(13)—N(1)—C(12)—O(2) | −6.0(7) |
| C(11)—O(2)—C(12)—O(3) | 178.7(5) |
| C(11)—O(2)—C(12)—N(1) | −0.8(6) |
| C(12)—N(1)—C(13)—C(11) | 9.5(7) |
| O(2)—C(11)—C(13)—N(1) | −9.1(6) |
| C(7)—C(11)—C(13)—N(1) | −130.7(5) |
| N(5)—N(2)—C(14)—N(3) | −178.0(4) |
| C(1)—N(2)—C(14)—N(3) | 10.8(8) |
| N(5)—N(2)—C(14)—C(17) | 0.8(5) |
| C(1)—N(2)—C(14)—C(17) | −170.4(4) |
| C(15)—N(3)—C(14)—N(2) | −178.6(4) |
| C(15)—N(3)—C(14)—C(17) | 2.8(7) |
| C(14)—N(3)—C(15)—N(4) | 1.1(7) |
| C(16)—N(4)—C(15)—N(3) | −2.2(8) |
| C(15)—N(4)—C(16)—N(6) | 179.6(5) |
| C(15)—N(4)—C(16)—C(17) | −0.4(6) |
| N(2)—C(14)—C(17)—C(18) | −1.2(5) |
| N(3)—C(14)—C(17)—C(18) | 177.6(5) |
| N(2)—C(14)—C(17)—C(16) | 176.0(4) |
| N(3)—C(14)—C(17)—C(16) | −5.3(7) |
| N(6)—C(16)—C(17)—C(14) | −176.3(5) |
| N(4)—C(16)—C(17)—C(14) | 3.7(6) |
| N(6)—C(16)—C(17)—C(18) | −0.4(9) |
| N(4)—C(16)—C(17)—C(18) | 179.6(5) |
| N(2)—N(5)—C(18)—C(17) | −0.8(5) |
| N(2)—N(5)—C(18)—C(19) | 178.4(5) |
| C(14)—C(17)—C(18)—N(5) | 1.2(5) |
| C(16)—C(17)—C(18)—N(5) | −175.0(5) |
| C(14)—C(17)—C(18)—C(19) | −177.8(6) |
| C(16)—C(17)—C(18)—C(19) | 5.9(10) |
| C(34)—N(22)—C(21)—C(23) | −121.2(6) |
| N(25)—N(22)—C(21)—C(23) | 68.1(6) |
| C(34)—N(22)—C(21)—C(22) | 113.9(6) |
| N(25)—N(22)—C(21)—C(22) | −56.8(6) |
| N(22)—C(21)—C(23)—C(28) | 115.8(5) |
| C(22)—C(21)—C(23)—C(28) | −121.3(6) |
| N(22)—C(21)—C(23)—C(24) | −65.8(6) |
| C(22)—C(21)—C(23)—C(24) | 57.2(7) |
| C(28)—C(23)—C(24)—C(25) | −1.5(7) |
| C(21)—C(23)—C(24)—C(25) | 180.0(5) |
| C(23)—C(24)—C(25)—C(26) | −0.7(8) |
| C(23)—C(24)—C(25)—Cl(21) | 178.6(4) |
| C(24)—C(25)—C(26)—F(21) | −179.6(4) |
| Cl(21)—C(25)—C(26)—F(21) | 1.0(7) |
| C(24)—C(25)—C(26)—C(27) | 1.2(8) |
| Cl(21)—C(25)—C(26)—C(27) | −178.2(4) |
| F(21)—C(26)—C(27)—C(28) | −178.5(4) |
| C(25)—C(26)—C(27)—C(28) | 0.7(7) |
| F(21)—C(26)—C(27)—C(31) | 5.0(7) |
| C(25)—C(26)—C(27)—C(31) | −175.8(5) |
| C(29)—O(21)—C(28)—C(23) | 94.0(6) |
| C(29)—O(21)—C(28)—C(27) | −87.6(6) |
| C(24)—C(23)—C(28)—O(21) | −178.1(4) |
| C(21)—C(23)—C(28)—O(21) | 0.4(6) |
| C(24)—C(23)—C(28)—C(27) | 3.5(7) |
| C(21)—C(23)—C(28)—C(27) | −178.0(4) |
| C(26)—C(27)—C(28)—O(21) | 178.6(4) |
| C(31)—C(27)—C(28)—O(21) | −5.0(6) |
| C(26)—C(27)—C(28)—C(23) | −3.1(6) |
| C(31)—C(27)—C(28)—C(23) | 173.4(4) |
| C(28)—O(21)—C(29)—C(30) | −148.9(7) |
| C(32)—O(22)—C(31)—C(27) | 128.3(5) |
| C(32)—O(22)—C(31)—C(33) | 5.2(6) |
| C(26)—C(27)—C(31)—O(22) | −58.3(6) |
| C(28)—C(27)—C(31)—O(22) | 125.4(5) |
| C(26)—C(27)—C(31)—C(33) | 59.8(6) |
| C(28)—C(27)—C(31)—C(33) | −116.5(5) |
| C(33)—N(21)—C(32)—O(23) | 173.6(6) |
| C(33)—N(21)—C(32)—O(22) | −5.7(7) |
| C(31)—O(22)—C(32)—O(23) | −179.5(5) |
| C(31)—O(22)—C(32)—N(21) | −0.1(6) |
| C(32)—N(21)—C(33)—C(31) | 8.4(7) |
| O(22)—C(31)—C(33)—N(21) | −7.7(6) |
| C(27)—C(31)—C(33)—N(21) | −129.0(5) |
| N(25)—N(22)—C(34)—N(23) | −179.0(5) |
| C(21)—N(22)—C(34)—N(23) | 9.5(8) |
| N(25)—N(22)—C(34)—C(37) | 0.3(5) |
| C(21)—N(22)—C(34)—C(37) | −171.2(4) |
| C(35)—N(23)—C(34)—N(22) | −178.9(5) |
| C(35)—N(23)—C(34)—C(37) | 2.0(8) |
| C(34)—N(23)—C(35)—N(24) | 0.8(8) |
| C(36)—N(24)—C(35)—N(23) | −1.1(8) |
| C(35)—N(24)—C(36)—N(26) | 178.0(5) |
| C(35)—N(24)—C(36)—C(37) | −1.3(7) |
| N(22)—C(34)—C(37)—C(36) | 176.5(4) |
| N(23)—C(34)—C(37)—C(36) | −4.2(7) |
| N(22)—C(34)—C(37)—C(38) | −0.8(5) |
| N(23)—C(34)—C(37)—C(38) | 178.5(5) |
| N(26)—C(36)—C(37)—C(34) | −175.7(5) |
| N(24)—C(36)—C(37)—C(34) | 3.5(6) |
| N(26)—C(36)—C(37)—C(38) | 0.4(9) |
| N(24)—C(36)—C(37)—C(38) | 179.6(5) |
| N(22)—N(25)—C(38)—C(37) | −0.8(5) |
| N(22)—N(25)—C(38)—C(39) | 179.9(5) |
| C(34)—C(37)—C(38)—N(25) | 1.0(5) |
| C(36)—C(37)—C(38)—N(25) | −175.3(5) |
| C(34)—C(37)—C(38)—C(39) | −179.8(5) |
| C(36)—C(37)—C(38)—C(39) | 3.9(10) |

### Example A1. PI3K Enzyme Assay

PI3-Kinase luminescent assay kit including lipid kinase substrate, D-myo-phosphatidylinositol 4,5-bisphosphate (PtdIns(4,5)P2)D (+)-sn-1,2-di-O-octanoylglyceryl, 3-O-phospho linked (PIP2), biotinylated I(1,3,4,5)P4, PI(3,4,5)P3 Detector Protein is purchased from Echelon Biosciences (Salt Lake City, Utah). AlphaScreen™ GST Detection Kit including donor and acceptor beads was purchased from PerkinElmer Life Sciences (Waltham, Mass.). PI3Kδ (p110δ/p85α) is purchased from Millipore (Bedford, Mass.). ATP, MgCl₂, DTT, EDTA, HEPES and CHAPS are purchased from Sigma-Aldrich (St. Louis, Mo.).

### AlphaScreen™ Assay for PI3Kδ

The kinase reaction are conducted in 384-well REMP plate from Thermo Fisher Scientific in a final volume of 40 μL. Inhibitors are first diluted serially in DMSO and added to the plate wells before the addition of other reaction components. The final concentration of DMSO in the assay is 2%. The PI3K assays are carried out at room temperature in 50 mM HEPES, pH 7.4, 5 mM MgCl₂, 50 mM NaCl, 5 mM DTT and CHAPS 0.04%. Reactions are initiated by the addition of ATP, the final reaction mixture consisted of 20 μM PIP2, 20 μM ATP, 1.2 nM PI3Kδ are incubated for 20 minutes. 10 μL of reaction mixture are then transferred to 5 μL 50 nM biotinylated I(1,3,4,5)P4 in quench buffer: 50 mM HEPES pH 7.4, 150 mM NaCl, 10 mM EDTA, 5 mM DTT, 0.1% Tween-20, followed with the addition of 10 μL AlphaScreen™ donor and acceptor beads suspended in quench buffer containing 25 nM PI(3,4,5)P3 detector protein. The final concentration of both donor and acceptor beads is 20 mg/ml. After plate sealing, the plate are incubated in a dark location at room temperature for 2 hours. The activity of the product is determined on Fusion-alpha microplate reader (Perkin-Elmer). IC₅₀ determination is performed by fitting the curve of percent control activity versus the log of the inhibitor concentration using the GraphPad Prism 3.0 software.

### Example A2: PI3K Enzyme Assay

Materials: Lipid kinase substrate, phosphoinositol-4,5-bisphosphate (PIP2), are purchased from Echelon Biosciences (Salt Lake City, Utah). PI3K isoforms α, β, δ and γ are purchased from Millipore (Bedford, Mass.). ATP, MgCl₂, DTT, EDTA, MOPS and CHAPS are purchased from Sigma-Aldrich (St. Louis, Mo.).

The kinase reactions are conducted in clear-bottom 96-well plate from Thermo Fisher Scientific in a final volume of 24 μL. Inhibitors are first diluted serially in DMSO and added to the plate wells before the addition of other reaction components. The final concentration of DMSO in the assay is 0.5%. The PI3K assays are carried out at room temperature in 20 mM MOPS, pH 6.7, 10 mM MgCl₂, 5 mM DTT and CHAPS 0.03%. The reaction mixture is prepared containing 50 μM PIP2, kinase and varying concentration of inhibitors. Reactions are initiated by the addition of ATP containing 2.2 μCi [γ-³³P]ATP to a final concentration of 1000 μM. The final concentration of PI3K isoforms α, β, δ and γ in the assay were 1.3, 9.4, 2.9 and 10.8 nM, respectively. Reactions are incubated for 180 minutes and terminated by the addition of 100 μL of 1 M potassium phosphate pH 8.0, 30 mM EDTA quench buffer. A 100 μL aliquot of the reaction solution are then transferred to 96-well Millipore MultiScreen IP 0.45 m PVDF filter plate (The filter plate is prewetted with 200 μL 100% ethanol, distilled water, and 1 M potassium phosphate pH 8.0, respectively). The filter plate is aspirated on a Millipore Manifold under vacuum and washed with 18×200 μL wash buffer containing 1 M potassium phosphate pH 8.0 and 1 mM ATP. After drying by aspiration and blotting, the plate is air dried in an incubator at 37° C. overnight. Packard TopCount adapter (Millipore) is then attached to the plate followed with addition of 120 μL Microscint 20 scintillation cocktail (Perkin Elmer) in each well. After the plate sealing, the radioactivity of the product is determined by scintillation counting on Topcount (Perkin-Elmer). IC₅₀ determination is performed by fitting the curve of percent control activity versus the log of the inhibitor concentration using the GraphPad Prism 3.0 software.

### Example A3. PI3Kδ Scintillation Proximity Assay

### Materials

[γ-³³P]ATP (10 mCi/mL) was purchased from Perkin-Elmer (Waltham, Mass.). Lipid kinase substrate, D-myo-Phosphatidylinositol 4,5-bisphosphate (PtdIns(4,5)P2)D (+)-sn-1,2-di-O-octanoylglyceryl, 3-O-phospho linked (PIP2), CAS 204858-53-7, was purchased from Echelon Biosciences (Salt Lake City, Utah). PI3Kδ (p110δ/p85α) was purchased from Millipore (Bedford, Mass.). ATP, MgCl₂, DTT, EDTA, MOPS and CHAPS were purchased from Sigma-Aldrich (St. Louis, Mo.). Wheat Germ Agglutinin (WGA) YSi SPA Scintillation Beads was purchased from GE healthcare life sciences (Piscataway, N.J.).

The kinase reaction is conducted in polystyrene 384-well matrix white plate from Thermo Fisher Scientific in a final volume of 25 μL. Inhibitors are first diluted serially in DMSO and added to the plate wells before the addition of other reaction components. The final concentration of DMSO in the assay is 0.5%. The PI3K assays are carried out at room temperature in 20 mM MOPS, pH 6.7, 10 mM MgCl₂, 5 mM DTT and CHAPS 0.03%. Reactions are initiated by the addition of ATP, the final reaction mixture consisted of 20 μM PIP2, 20 μM ATP, 0.2 μCi [γ-³³P] ATP, 4 nM PI3Kδ. Reactions are incubated for 210 min and terminated by the addition of 40 μL SPA beads suspended in quench buffer: 150 mM potassium phosphate pH 8.0, 20% glycerol. 25 mM EDTA, 400 μM ATP. The final concentration of SPA beads is 1.0 mg/mL. After the plate sealing, plates are shaken overnight at room temperature and centrifuged at 1800 rpm for 10 minutes, the radioactivity of the product is determined by scintillation counting on Topcount (Perkin-Elmer). IC₅₀ determination is performed by fitting the curve of percent control activity versus the log of the inhibitor concentration using the GraphPad Prism 3.0 software.

### Example B1. B Cell Proliferation Assay

To acquire B cells, human PBMC are isolated from the peripheral blood of normal, drug free donors by standard density gradient centrifugation on Ficoll-Hypague (GE Healthcare, Piscataway, N.J.) and incubated with anti-CD19 microbeads (Miltenyi Biotech, Auburn, Calif.). The B cells are then purified by positive immunosorting using an autoMacs (Miltenyi Biotech) according to the manufacture's instruction.

The purified B cells (2×10⁵/well/200 μL) are cultured in 96-well ultra-low binding plates (Corning, Corning, N.Y.) in RPMI1640, 10% FBS and goat F(ab′)2 anti-human IgM (10 μg/ml) (Invitrogen, Carlsbad, Calif.) in the presence of different amount of test compounds for three days. [3H]-thymidine (1 ρCi/well) (PerkinElmer, Boston, Mass.) in PBS is then added to the B cell cultures for an additional 12 hours before the incorporated radioactivity is separated by filtration with water through GF/B filters (Packard Bioscience, Meriden, Conn.) and measured by liquid scintillation counting with a TopCount (Packard Bioscience).

### Example B2. Pfeiffer Cell Proliferation Assay

Pfeiffer cell line (diffuse large B cell lymphoma) are purchased from ATCC (Manassas, Va.) and maintained in the culture medium recommended (RPMI and 10% FBS). To measure the anti-proliferation activity of the compounds, the Pfeiffer cells are plated with the culture medium (2×10³ cells/well/per 200 μl) into 96-well ultra-low binding plates (Corning, Corning, N.Y.), in the presence or absence of a concentration range of test compounds. After 3-4 days, [3H]-thymidine (1 μCi/well) (PerkinElmer, Boston, Mass.) in PBS is then added to the cell culture for an additional 12 hours before the incorporated radioactivity is separated by filtration with water through GF/B filters (Packard Bioscience, Meridenj, CT) and measured by liquid scintillation counting with a TopCount (Packard Bioscience).

### Example B3. SUDHL-6 cell proliferation assay

SUDHL-6 cell line (diffuse large B cell lymphoma) is purchased from ATCC (Manassas, Va.) and maintained in the culture medium recommended (RPMI and 10% FBS). To measure the anti-proliferation activity of the compounds through ATP quantitation, the SUDHL-6 cells is plated with the culture medium (5000 cells/well/per 200 μl) into 96-well polystyrene clear black tissue culture plate (Greiner-bio-one through VWR, NJ) in the presence or absence of a concentration range of test compounds. After 3 days, Cell Titer-GLO Luminescent (Promega, Madison, Wis.) cell culture agent is added to each well for 10 minutes at room temperature to stabilize the luminescent signal. This determines the number of viable cells in culture based on quantitation of the ATP present, which signals the presence of metabolically active cells. Luminescence is measured with the TopCount 384 (Packard Bioscience through Perkin Elmer, Boston, Mass.).

### Example C. Akt Phosphorylation Assay

Ramos cells (B lymphocyte from Burkitts lymphoma) are obtained from ATCC (Manassas, Va.) and maintained in RPMI1640 and 10% FBS. The cells (3×10′ cells/tube/3 mL in RPMI) are incubated with different amounts of test compounds for 2 hrs at 37° C. and then stimulated with goat F(ab′)2 anti-human IgM (5 μg/mL) (Invitrogen) for 17 minutes in a 37° C. water bath. The stimulated cells are spun down at 4° C. with centrifugation and whole cell extracts are prepared using 300 μL lysis buffer (Cell Signaling Technology, Danvers, Mass.). The resulting lysates are sonicated and supernatants are collected. The phosphorylation level of Akt in the supernatants are analyzed by using PathScan phospho-Akt1 (Ser473) sandwich ELISA kits (Cell Signaling Technology) according to the manufacturer's instruction.

Various modifications of the invention, in addition to those described herein, will be apparent to those skilled in the art from the foregoing description. Such modifications are also intended to fall within the scope of the appended claims. Each reference, including all patent, patent applications, and publications, cited in the present disclosure is incorporated herein by reference in its entirety.

1. A salt, which is selected from:
(R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one hydrochloric acid salt;
(R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one phosphoric acid salt;
(R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one maleic acid salt; and
(R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one p-toluenesulfonic acid salt.

2. The salt of claim 1, that is crystalline.

3. (canceled)

4. The salt of claim 1, that is a crystalline (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one hydrochloric acid salt.

5. The salt of claim 4, wherein the salt comprises a 1:1 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to hydrochloric acid

6. The salt of claim 4, having at least one XRPD peak, in terms of 2-theta, selected from about 10.2°, about 10.7°, about 14.7°, about 18.2°, about 19.6°, about 19.9°, about 20.5°, about 21.5°, about 22.0°, about 22.3°, and about 26.4°.

7-11. (canceled)

12. The salt of claim 4, having an XRPD profile substantially as shown in FIG. 1.

13. (canceled)

14. The salt of claim 4, having a DSC thermogram substantially as shown in FIG. 2.

15. The salt of claim 4, having a TGA thermogram substantially as shown in FIG. 3.

16. (canceled)

17. The salt of claim 1, that is a crystalline (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one phosphoric acid salt.

18. The salt of claim 17, wherein the salt comprises a 5:4 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to phosphoric acid.

19. The salt of claim 17, having at least one XRPD peak, in terms of 2-theta, selected from about 11.1°, about 11.3°, about 15.6°, about 17.7°, about 18.1°, about 18.3°, about 18.6°, about 21.1°, about 22.3°, about 22.9°, about 23.5°, about 23.7°, and about 25.1°.

20-24. (canceled)

25. The salt of claim 17, having an XRPD profile substantially as shown in FIG. 4.

26. (canceled)

27. The salt of claim 17, having a DSC thermogram substantially as shown in FIG. 5.

28. The salt of claim 17, having a TGA thermogram substantially as shown in FIG. 6.

29. (canceled)

30. The salt of claim 1, that is a crystalline (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one maleic acid salt.

31. The salt of claim 30, wherein the salt comprises a 1:1 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to maleic acid.

32. The salt of claim 30, having at least one XRPD peak, in terms of 2-theta, selected from about 11.1°, about 11.3°, about 15.6°, about 17.7°, about 18.1°, about 18.3°, about 18.6°, about 21.1°, about 22.3°, about 22.9°, about 23.5°, about 23.7°, and about 25.1°.

33-37. (canceled)

38. The salt of claim 30, having an XRPD profile substantially as shown in FIG. 7.

39. (canceled)

40. The salt of claim 30, having a DSC thermogram substantially as shown in FIG. 8.

41. The salt of claim 30, having a TGA thermogram substantially as shown in FIG. 9.

42. (canceled)

43. The salt of claim 1, that is a crystalline (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one p-toluenesulfonic acid salt.

44. The salt of claim 43, wherein the salt comprises a 1:1 stoichiometric ratio of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one to p-toluenesulfonic acid.

45. The salt of claim 43, having at least one XRPD peak, in terms of 2-theta, selected from about 8.8°, about 11.9°, about 17.0°, about 17.7°, about 22.4°, about 23.6°, and about 24.3°.

46-50. (canceled)

51. The salt of claim 43, having an XRPD profile substantially as shown in FIG. 10.

52. (canceled)

53. The salt of claim 43, having a DSC thermogram substantially as shown in FIG. 11.

54. The salt of claim 43, having a TGA thermogram substantially as shown in FIG. 12.

55. (canceled)

56. A crystalline solid form of (R)-5-(3-((S)-1-(4-amino-3-methyl-1H-pyrazolo[3,4-d]pyrimidin-1-yl)ethyl)-5-chloro-2-ethoxy-6-fluorophenyl)oxazolidin-2-one free base.

57. (canceled)

58. The crystalline solid form of claim 56, having at least one XRPD peak, in terms of 2-theta, selected from about 9.2°, about 11.5°, about 14.2°, about 15.1°, about 20.3°, about 20.7°, about 21.4°, about 23.0°, and about 27.6°.

59-63. (canceled)

64. The crystalline solid form of claim 56, having an XRPD profile substantially as shown in FIG. 13.

65. (canceled)

66. The crystalline solid form of claim 56, having a DSC thermogram substantially as shown in FIG. 14.

67. The crystalline solid form of claim 56, having a TGA thermogram substantially as shown in FIG. 15.

68. (canceled)

69. A pharmaceutical composition comprising a salt of claim 1, and a pharmaceutically acceptable carrier.

70. A method of inhibiting an activity of a PI3K kinase, comprising contacting the kinase with a salt of claim 1.

71-72. (canceled)

73. A method of treating a disease in a patient, wherein said disease is associated with abnormal expression or activity of a PI3K kinase, comprising administering to said patient a therapeutically effective amount of a salt of claim 1.

74-85. (canceled)

86. A process of preparing the salt of claim 4, comprising reacting a compound of Formula I: 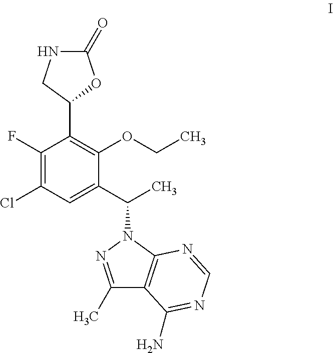 [SMILES: CCOc1c([C@H](C)n2nc(C)c3c(N)ncnc32)cc(Cl)c(F)c1[C@@H]1CNC(=O)O1]
with hydrochloric acid to form said salt.

87-89. (canceled)

90. A process of preparing the salt of claim 17, comprising reacting a compound of Formula I: 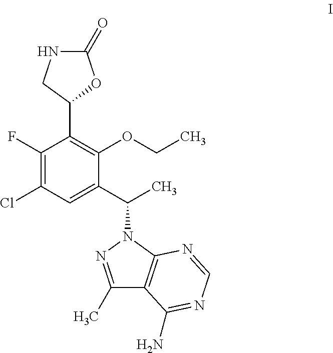 [SMILES: CCOc1c([C@H](C)n2nc(C)c3c(N)ncnc32)cc(Cl)c(F)c1[C@@H]1CNC(=O)O1]
with phosphoric acid to form said salt.

91-93. (canceled)

94. A process of preparing the salt of claim 30, comprising reacting a compound of Formula I: 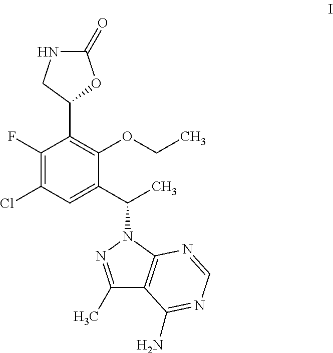 [SMILES: CCOc1c([C@H](C)n2nc(C)c3c(N)ncnc32)cc(Cl)c(F)c1[C@@H]1CNC(=O)O1]
with maleic acid to form said salt.

95-96. (canceled)

97. A process of preparing the salt of claim 43, comprising reacting a compound of Formula I: 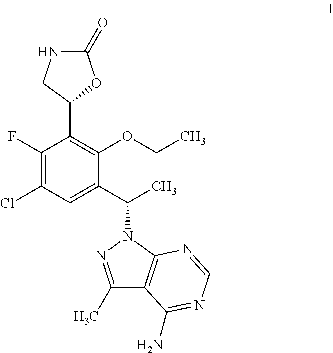 [SMILES: CCOc1c([C@H](C)n2nc(C)c3c(N)ncnc32)cc(Cl)c(F)c1[C@@H]1CNC(=O)O1]
with p-toluenesulfonic acid to form said salt.

98-99. (canceled)

100. A process comprising reacting a compound of Formula XIV: 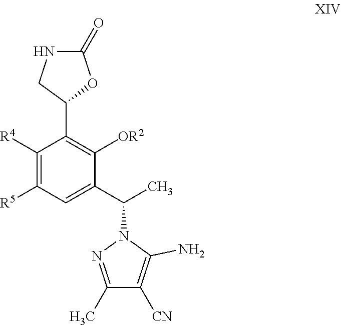
with formamidine acetate to form a compound of Formula IA: 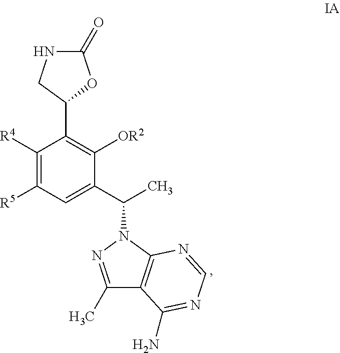
wherein:
R² is C₁₋₆ alkyl;
R⁴ is halo, CN, or C₁₋₃ alkyl; and
R⁵ is halo, CN, or C₁₋₃ alkyl.

101-103. (canceled)

104. The process of claim 100, wherein said compound of Formula XIV is prepared by a process comprising reacting a compound of Formula XIII: 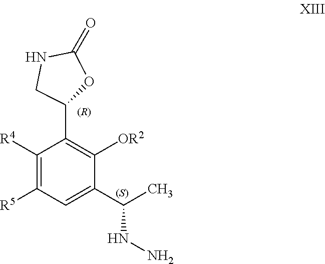
with (1-ethoxyethylidene)malononitrile.

105-107. (canceled)

108. The process of claim 104, wherein said compound of Formula XIII is prepared by a process comprising deprotecting a compound of Formula XII: 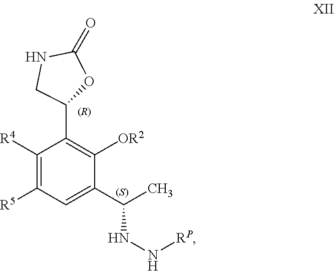
wherein R^{p} is an amine protecting group.

109-112. (canceled)

113. The process of claim 108, wherein said compound of Formula XII is prepared by a process comprising reacting a compound of Formula XI: 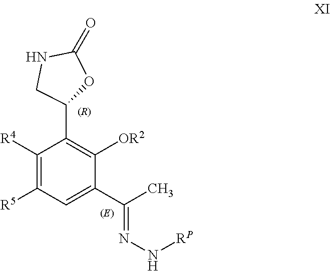
with hydrogen gas in the presence of one or more independently selected hydrogenation catalysts.

114-121. (canceled)

122. The process of claim 113, wherein said compound of Formula XI is prepared by a process comprising reacting a compound of Formula X: 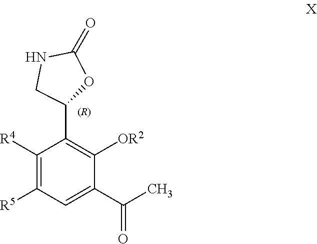
with R^{p}—NHNH₂, wherein R is an amine protecting group.

123-128. (canceled)

129. The process of claim 122, wherein said compound of Formula X is prepared by a process comprising reacting a compound of Formula TX: 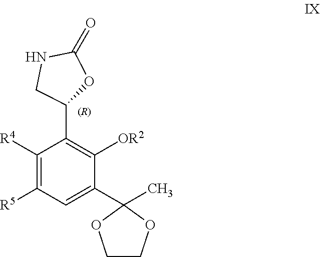
with an acid.

130-133. (canceled)

134. The process of claim 129, wherein said compound of Formula IX is prepared by a process comprising reacting a compound of Formula VIII: 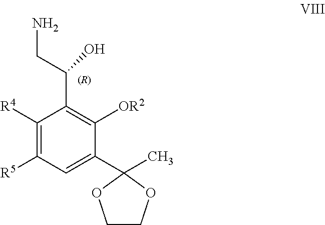
with carbonyldiimidazole.

135-137. (canceled)

138. The process of claim 134, wherein said compound of Formula VIII is prepared by a process comprising reacting a compound of Formula VII: 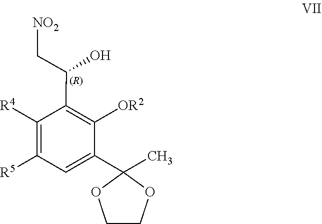
with hydrogen gas in the presence of one or more independently selected hydrogenation catalysts.

139-144. (canceled)

145. The process of claim 134, wherein said compound of Formula VIII is prepared by a process comprising reacting a compound of Formula VIII-rac 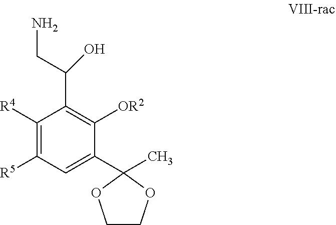
with an acidic chiral resolving agent.

146-150. (canceled)

151. The process of claim 145, wherein said compound of Formula VIII-rac is prepared by a process comprising reacting a compound of Formula VII-rac: 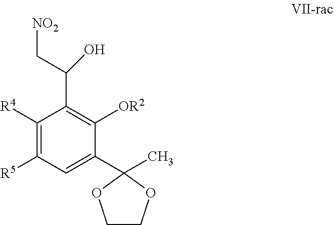
with hydrogen gas in the presence of one or more independently selected hydrogenation catalysts.

152-157. (canceled)

158. The process of claim 151, wherein said compound of Formula VII-rac is prepared by a process comprising reacting a compound of Formula VI: 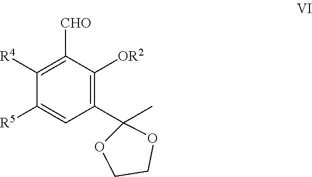
with nitromethane in the presence of a base.

159-163. (canceled)

164. The process of claim 138, wherein said compound of Formula VII is prepared by a process comprising reacting a compound of Formula VI: 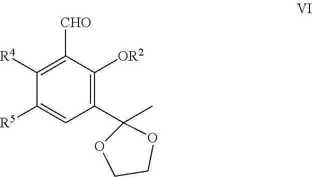
with nitromethane in the presence of a chiral catalyst, and an amine base.

165-172. (canceled)

173. The process of claim 158, wherein said compound of Formula VI is prepared by a process comprising reacting a compound of Formula V-a: 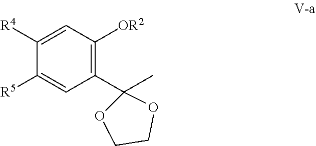
with N,N-dimethylformamide or N-formylmorpholine in the presence of lithium diisopropylamide.

174-178. (canceled)

179. The process of claim 173, wherein said compound of Formula V-a is prepared by a process comprising reacting a compound of Formula IV-c: 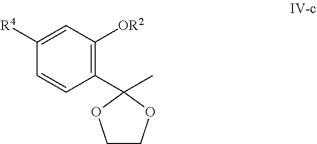
with a halogenating agent, a cyanating agent or an alkylating agent.

180-184. (canceled)

185. The process of claim 179, wherein said compound of Formula IV-c is prepared by a process comprising reacting a compound of Formula III-c: 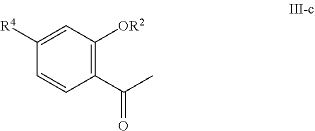
with 1,2-ethanediol in the presence of p-toluenesulfonic acid and triethyl orthoformate.

186-190. (canceled)

191. The process of claim 185, wherein said compound of Formula III-c is prepared by a process comprising reacting a compound of Formula II-c: 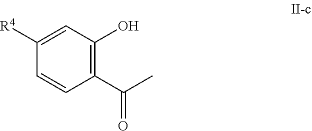
with R²—X¹ in the presence of an alkali metal carbonate base, wherein X¹ is halide.

192-202. (canceled)

203. A compound of any one of Formulas VII, VIII, IX, X, XI, XII, XIII, XIV, VII-rac, and VIII-rac: 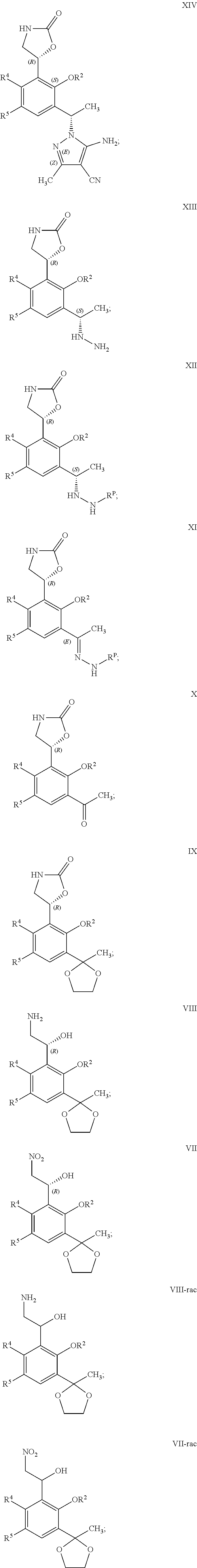
or a salt thereof, wherein:
R^{p} is an amine protecting group;
R² is C₁₋₆ alkyl;
R⁴ is halo, CN, or C₁₋₃ alkyl; and
R⁵ is halo, CN, or C₁₋₃ alkyl.

204-205. (canceled)

206. A pharmaceutical composition comprising a crystalline solid form of claim 56, and a pharmaceutically acceptable carrier.

207. A method of inhibiting an activity of a PI3K kinase, comprising contacting the kinase with a crystalline solid form of claim 56.

208. A method of treating a disease in a patient, wherein said disease is associated with abnormal expression or activity of a PI3K kinase, comprising administering to said patient a therapeutically effective amount of a crystalline solid form of claim 56.
